(19) United States
(12) Patent Application Publication
(10) Pub. No.: US 2026/0137719 A1
(43) Pub. Date: May 21, 2026

(54) ANTI-INFLAMMATORY RED BLOOD CELL EXTRACELLULAR VESICLES (RBCEVS)

(71) Applicant: NATIONAL UNIVERSITY OF SINGAPORE, Singapore (SG)

(72) Inventors: Minh Thi Nguyet LE, Singapore (SG); Tuan Thach PHAM, Singapore (SG); Cong Phi DANG, Singapore (SG); Jiong-Wei WANG, Singapore (SG)

(21) Appl. No.: 19/120,690

(22) PCT Filed: Oct. 11, 2023
(86) PCT No.: PCT/SG2023/050687

§ 371 Date: Apr. 11, 2025

Related U.S. Application Data
(60) Provisional application No. 63/415,250, filed on Oct. 11, 2022.

(51) Int. Cl. A61K 35/18; A61K 9/50; A61K 31/7088; A61K 31/7105; A61P 9/10

(52) U.S. Cl. CPC A61K 35/18; A61K 9/5068; A61K 31/7088; A61K 31/7105; A61P 9/10

(57) ABSTRACT

The present disclosure describes anti-inflammatory properties of red blood cell extracellular vesicles, e.g., as mediated by heme, hemoglobin and/or phosphatidylserine content. The present disclosure describes technologies useful for the treatment of inflammatory diseases, disorders, or conditions (e.g., atherosclerosis).

This application claims priority from U.S. 63/415,250 filed 11 Oct. 2022, the contents and elements of which are herein incorporated by reference for all purposes.

### BACKGROUND

Inflammatory diseases, disorders, and conditions pose a major challenge to global public health. For example, atherosclerosis is one of the leading causes of death and disability in the developed world.

### SUMMARY

The present disclosure provides certain technologies relating to treatment of inflammatory diseases, disorders, and conditions.

Inflammatory diseases, disorders, and conditions pose a major challenge to global public health. The most common inflammatory diseases, disorders, and conditions (e.g., atherosclerosis) are often associated with cellular dysfunction in certain populations (e.g., as defined by age, weight categorization, lifestyle and/or environment, etc.). The present disclosure provides technologies that can achieve anti-inflammatory effects for treatment and/or prevention (e.g., delay of onset or exacerbation, reduction in risk of onset or exacerbation, etc.) of such inflammatory diseases, disorders, or conditions.

Among other things, the present disclosure recognizes that certain extracellular vesicles, and particularly red blood cell extracellular vesicles (RBCEVs), can provide desirable anti-inflammatory effects. RBCEVs can provide such anti-inflammatory effects both when loaded with exogenous nucleic acid and when not loaded with exogenous nucleic acid. The present disclosure provides a method of treating and/or preventing an inflammatory disease, disorder, or condition in a human subject comprising administering to the subject a composition comprising a population of extracellular vesicles derived from red blood cells (RBCEVs). Also provided is a composition comprising a population of extracellular vesicles derived from red blood cells (RBCEVs) for use in a method of treating and/or preventing an inflammatory disease, disorder, or condition. Also provided is the use of a composition comprising a population of extracellular vesicles derived from red blood cells (RBCEVs) in the manufacture of a medicament for treating and/or preventing an inflammatory disease, disorder, or condition.

Advantages that can be achieved by provided technologies include, for example, low toxicity, low cost of production, lack of immunogenicity, lack of oncogenicity, easy accessibility, simple composition, high amenability for nucleic acid loading (specifically including of long nucleic acids and/or single stranded nucleic acids, and/or RNAs).

Among other things, the present disclosure documents that certain extracellular vesicles, and particularly red blood cell extracellular vesicles (RBCEVs), can achieve successful prevention and/or amelioration of inflammatory diseases, disorders, and conditions.

The present disclosure documents cell type-dependent uptake of RBCEVs, e.g., preferential uptake by monocytes and/or macrophages in vitro and in vivo.

The present disclosure documents internalization of RBCEVs in macrophages to mainly be through endocytosis in a process mediated by phosphatidylserine, and accumulate primarily in late endosome and lysosomes.

The present disclosure documents induction of an Mheme-like phenotype in peripheral blood mononuclear cells (PBMCs) when contacted with RBCEVs (e.g., RBCEVs containing heme and/or hemoglobin), for example, as characterized by reduced CD86 expression.

The present disclosure documents reduction of foam cell formation in macrophages when contacted with RBCEVs, for example, as characterized by oil red O staining.

The present disclosure documents a method, composition for use or use, wherein the RBCEVs comprise heme, hemoglobin and/or phosphatidylserine.

The present disclosure documents a method, composition for use or use, wherein the RBCEVs are not loaded with exogenous nucleic acid.

The present disclosure documents a method, composition for use or use, wherein the RBCEVs are loaded with exogenous nucleic acid.

In methods, compositions for use or uses of the present disclosure, exogenous nucleic acid may be or may comprise an siRNA or an ASO.

In methods, compositions for use or uses of the present disclosure, exogenous nucleic acid may be or may comprise an siRNA or an ASO for the gene knockdown of VEGF.

The present disclosure documents a method, composition for use or use, wherein the RBCEVs are loaded with exogenous nucleic acid that is or comprises an siRNA or an ASO for the gene knockdown of VEGF.

The present disclosure documents a method, composition for use or use, wherein the inflammatory disease, disorder, or condition to be treated or prevented is or comprises atherosclerosis.

The present disclosure documents a method, composition for use or use, characterized in that the administration of the composition comprising a population of RBCEVs is associated with reduced levels of one or more inflammatory cytokines.

In methods, compositions for use or uses of the present disclosure, inflammatory cytokines may be selected from the group consisting of TNF-α, IL-6, and IL-12.

The present disclosure documents a method, composition for use or use, characterized in that the administration of the composition comprising a population of RBCEVs is associated with reduced levels of one or more inflammatory cytokines selected from the group consisting of TNF-α, IL-6, and IL-12.

The present disclosure documents a method, composition for use or use, characterized in that the administration of the composition comprising a population of RBCEVs is associated with reduced formation of foam cells.

The present disclosure documents a method, composition for use or use, characterized in that the administration of the composition comprising a population of RBCEVs is associated with increased induction of Mheme-like phenotype in macrophages.

The present disclosure documents a pharmaceutical composition comprising a population of extracellular vesicles derived from red blood cells (RBCEVs) for the treatment and/or prevention of atherosclerosis.

### BRIEF DESCRIPTION OF THE DRAWINGS

FIG. 1. RBCEVs are taken up preferably by macrophages and monocytes. Panel A. Schematic illustrating experimental setup to track biodistribution of RBCEVs using Acoerela dyes. Panel B. Confocal images of liver and spleen sections stained with antibodies against macrophage markers (F4/80 or CD169) (Red). Nuclei were stained with NucSpot® Live 488 (Cyan). RBCEVs were labeled with Acoerela Aco-490 (Green) and 500 μg of RBCEVs were injected intravenously in C57BL/6 mice. Organs were collected 8 hours after injection, fixed in formalin overnight and snap frozen for cryo-sectioning. Scale bar: 200 μm. Panel C. Flow cytometry analysis of Aco-490 signals in PBMCs. Cells were incubated with Aco-490-labeled RBCEVs for 2 hours or 24 hours and then harvested and stained with antibodies for different surface markers. Panel D. Number of RBCEVs taken up by different cell types including cancer cell lines and M0 macrophages after being incubated with 40 μg of RBCEVs for 2 hours. Results were obtained using an absolute quantification method with CFSE-labeled EVs. Panel E. Flow cytometry analysis quantifying RBCEV uptake by different types of macrophages. Macrophages were differentiated from human CD14+ PBMCs in M-CSF for 6 days (M0) and stimulated with LPS and INF-γ for 1 day (M1) or stimulated with IL-4 and IL-10 for 1 day (M2). Subsequently, cells were incubated with CFSE-labeled RBCEVs for 2 hours and collected for flow cytometry analysis. a.u: arbitrary unit. All bar graphs represent mean±SD.

FIG. 2. Uptake of RBCEVs by macrophages is mediated by phosphatidylserine. Panel A. Flow cytometry analysis of RBCEV uptake by PBMC-derived macrophages that were pre-incubated with phosphatidylserine (PS) liposomes or phosphatidylcholine (PC) liposomes at different concentrations for 30 mins. Cells were incubated with CFSE-labeled RBCEVs for 2 hours. Panel B. Nanoparticle flow cytometry (NanoFCM) analysis of Annexin V staining of phosphatidylserine (PS) on RBCEVs' surface. RBCEVs were labeled with CFSE and then treated with α-cyclodextrin and 1,2-distearoyl-sn-glycero-3-phosphocholine (DSPC) to reduce PS on their outer leaflet membrane (PS reduced). L-α-phosphatidylserine was added to PS-reduced EVs (PS restored) to restore PS expression. Untreated RBCEVs and modified RBCEVs were stained with Annexin V for PS detection. Upper panel shows controls and gating strategy. Subsequently, Annexin V was gated based on CFSE-positive particles. Panel C. Flow cytometry analysis of CFSE indicating uptake of RBCEVs treated as in Panel B by macrophages. After PS removal or PS restoration, RBCEVs were incubated with macrophages for 2 hours, and then cells were harvested and analyzed for CFSE signals using flow cytometry. Student's two-tailed t-test, ** p<0.01, ns: not significant. a.u: arbitrary unit. All bar graphs represent mean±SD.

FIG. 3. Uptake of RBCEVs by macrophages is an active process and mainly mediated by endocytosis. Panel A. Flow cytometry analysis of CFSE indicating uptake of CFSE-labeled RBCEVs by macrophages at 4° C. and 37° C. after 2 hours of incubation. Panel B. Flow cytometry analysis of CFSE indicating uptake of RBCEVs by macrophages in a time-dependent and concentration-dependent manner. Macrophages were incubated with 20 μg of CFSE-labeled RBCEVs for different durations of time (right) or incubated for 2 hours with different amounts of EVs (left). Panel C. Different routes of endocytosis and inhibitors which can block them. Filipin blocks lipid raft and caveolin-mediated endocytosis. EIPA inhibits macropinocytosis. Wortmannin prevents phagocytosis and macropinocytosis. Cytochalasin D disrupts actin filaments and hence prevents all actin-mediated plasma-membrane modulation and affects all endocytosis routes including phagocytosis. Panel D. Flow cytometry analysis of CFSE indicating uptake of CFSE-labeled RBCEVs by macrophages after treatment with different endocytosis inhibitors including Cytochalasin D, EIPA, Filipin, and Wortmannin. Cells were treated with different concentrations of each inhibitor for 1 hour, followed by incubation with 20 μg of CFSE-labeled EVs for 1 hour, and then subjected to flow cytometry analysis. Panel E. Flow cytometry analysis of GPA on macrophages treated with RBCEVs for 2 hours at 4° C. and 37° C. Student's two-tailed t-test, *p<0.05, ns: not significant. Panel F. Immunofluorescence images of macrophages stained with CFSE or CellTrace Far Red (CTFR) and incubated with RBCEVs to observe fusion events. Labeled cells, 100,000 of each type, were mixed with 20 μg of RBCEVs and seeded in each well of a 96-well plate, centrifuged at 500×g for 20 mins at RT, and incubated overnight at 37° C. Cells were then trypsinized, fixed, stained with Hoechst and mounted on slides for imaging. Quantification determines the number of fusion events (cells with two or more nuclei) over 1000-2000 cells. a.u: arbitrary unit. All bar graphs represent mean±SD.

FIG. 4. RBCEVs accumulate in late endosomes and lysosomes. Panel A. Experimental schema for tracking intracellular trafficking of RBCEVs in macrophages. Panel B. Confocal images of macrophages after a 2-hour exposure to CFSE-labeled RBCEVs. Cells were co-stained with antibodies for early endosomal marker (EEA), late endosomal marker (LBPA), and late endosomal-lysosomal marker (LAMP-1). Nuclei were stained with Hoechst. Scale bar 20 μm. Panel C. Pearson correlation coefficient indicating colocalization of CFSE with endosomal markers over time. Images were analyzed using ImageJ. Panel D. Confocal images of macrophages at different time points (30 mins, 2 h, 4 h, and 24 h) after a 2-hour exposure to CFSE-labeled RBCEVs. Cells were stained with antibody against alpha hemoglobin. Nuclei were stained with Hoechst. Scale bar: 10 μm. Panel E. Quantification of hemoglobin signals in macrophages incubated with RBCEVs over time based on confocal imaging. a.u: arbitrary unit. All bar graphs represent mean±SD.

FIG. 5. RBECVs promote differentiation of macrophages into an Mheme-like phenotype. Panel A. Relative mRNA expression levels (normalized to GAPDH) of Heme oxygenase 1 (HO-1), LXRb, ABCA1, and ABCG1 and of cytokines IL-10, IL-1b, and TNFα in macrophages differentiated from CD14+ PBMCs. Four groups of cells including untreated group, control group treated with haptoglobin-hemoglobin complexes, and two RBCEV treated groups with different EV concentrations (100 ng/μl and 120 ng/μL) were collected and subjected to qPCR after 7 days. Two-way ANOVA test, **** p<0.0001, ** p<0.01, *p<0.05. Panel B. Flow cytometry analysis of M1 macrophage makers (CD80 and CD68), M2 macrophage marker (MMR, also called CD206), and Mheme macrophage markers (CD206 and CD163). All markers were gated from CD11b+ cells. Macrophages were differentiated in M-CSF (M0) and incubated with RBCEVs (80 ng/μL or 160 ng/L) for 8 days (M0-EV). RBCEV-treated macrophages were compared with M1 macrophages (activated by LPS and IFN-γ), M2 macrophages (activated by IL4 and IL-10), and Mheme macrophages (stimulated by haptoglobin-hemoglobin). Panel C. ELISA quantification of pro-inflammatory cytokines TNF-α, IL-6, and IL-12 in supernatant of macrophages treated with RBCEVs and challenged with LPS or medium only for 24 hours. Student's two-tailed t-test, *p<0.05, ns: not significant. a.u: arbitrary unit. All bar graphs represent mean±SD.

FIG. 6. Hemoglobin carried by RBCEVs induces macrophages into an Mheme-like phenotype. Panel A. Schematic illustration of experimental setup for qPCR and flow cytometry analysis of RBCEVs and RBCEV ghost induced macrophages. Panel B. Quantification of hemoglobin content in RBCEV ghosts. Hemoglobin content in RBCEV ghosts is relative to amount of hemoglobin in original RBCEVs. Panel C. qPCR analysis of HO-1, LXRb, ABCA1, ABCG1, and IL-10 mRNA level in different groups of macrophages differentiated from CD14+ PBMCs. Macrophages were differentiated in M-CSF (M0) and incubated with RBCEV ghosts or RBCEVs (in similar number of vesicles, equivalent to 160 ng/μL RBCEVs) for 7 days. Two-way ANOVA test, **** p<0.0001, *p<0.05, ns: not significant. Panel D. Flow cytometry analysis of M1, M2 and Mheme macrophage markers (CD80, CD86, CD163 and CD206 (MMR)). All markers were analysed on CD11b+ cell population. Macrophages were differentiated in M-CSF (M0) and incubated with RBCEV ghosts or RBCEVs (in similar number of vesicles, equivalent to 160 ng/μL RBCEVs) for 7 days. a.u: arbitrary unit. All bar graphs represent mean±SD.

FIG. 7. HRG-1 mediates upregulation of HO-1 by RBCEV treatment. Panel A. qPCR analysis of HRG1 mRNA expression (normalized to GAPDH) in macrophages transfected with HRG1 ASO for 48 hours and either untreated or treated with RBCEVs for the last 24 hours (n=4). Panel B. Western blot analysis of HRG1 protein in macrophages transfected with HRG-1 ASOs for 72 hours (n=3). Panel C. qPCR analysis of relative HO-1 mRNA levels (normalized to GAPDH) in macrophages transfected with HRG1 ASO for 48 hours and either untreated or treated with RBCEVs for the last 24 hours (n=4). All bar graphs represent mean±SD. Panel D. Confocal images of macrophages after a 2-hour exposure to RBCEVs. The cells were immunolabeled with antibodies against the RBCEV marker BAND 3 (Green) and HRG1 (Magenta). The nuclei were then counterstained with Hoechst 33342 (Cyan). The white arrows indicate HRG1 clusters. Scale bar, 10 μm. Panels E-F. Quantification of the of HRG1 clusters (E) and HRG1 signals (F) per cell from images as shown in (D) (n=12 for ‘Untreated, n=24 for ‘RBCEVs’). Panel G. Confocal images of macrophages after a 2-hour exposure to CFSE-labeled RBCEVs and analysis of co-localization between CFSE (Green), HRG1 (Magenta), and LAMP1 (Red) signals. The cells were double immunolabeled with antibodies against LAMP1 and HRG1 and the nuclei were counterstained with Hoechst 33342 (Cyan). Scale bar, 10 μm. Panel H. Mander's overlap coefficient analysis of HRG1 signals and LAMP1 signals from images as shown in (G) (n=17 for ‘Untreated’, n=31 for ‘RBCEVs’). Each data point corresponds to one cell in (E, F, H). Panel I. Representative signal intensity profiles of CFSE, LAMP1 and HRG1 (middle panels) obtained along the solid white line in the merged images (left panels), and Mander's overlap coefficient analysis of CFSE-LAMP1 double positive signals and HRG1 signals (right panel). Images were analyzed with ImageJ. Student's two-tailed t-test (a,b,c,e,f,h), *** p<0.001, ** p<0.01, *p<0.05. a.u.: arbitrary unit.

FIG. 8. RBCEVs prevent macrophage foam cell formation. Panel A. Representative images of macrophages stained with oil red O indicating foam cell formation. Monocyte-derived macrophages were seeded on cover slips and treated with RBCEVs or haptoglobin-hemoglobin complexes for 7 days. Cells were subsequently incubated with human oxidized low-density lipoprotein (oxLDL) or medium only for 24 hours before oil red O staining. Panel B. Quantification of oil red O staining amongst groups of macrophages treated as in Panel A. Relative fold change in level of staining was calculated by normalization to oxLDL-treated control group. Oil red O signal intensity per cell was analyzed using ImageJ software. Data were collected from PBMC-derived macrophages of 3-4 donors. Panel C. Representative images of M1 macrophages stained with oil red O. Monocyte-derived macrophages were seeded on cover slips and incubated with RBCEVs for 7 days, followed by incubation with 0.5 mM EDTA for 24 hours. Then cells were washed and incubated with LPS and human oxLDL for 24 hours to activate M1 phenotype and induce foam cell formation, respectively. Panel D. Quantification of oil red O staining that indicates the level of foam cell formation amongst groups of activated macrophages. Data are presented as relative fold change in level of oil red O staining intensity of PBMC-derived macrophages from 3-4 donors. All bar graphs represent mean±SD. Two-way ANOVA test (b, d), **** p<0.0001, *** p<0.001, ** p<0.01.

FIG. 9. RBCEVs prevent atherosclerosis in a high-fat diet ApoE knockout mouse model. Panel A. Schematic illustrating the experimental setup to test the effect of RBCEVs on atherosclerosis using a high-fat diet ApoE knockout (ApoE−/−) mouse model. The treated group was injected with 50 mg/kg RBCEVs in 100 μL of PBS, while the control group was injected with the same volume of PBS only. i.v.: intravenous injection. Panel B. Aortic arches of mice after the course of the 8-week treatment described in A. White spots and streaks on the inner wall of the aorta are atherosclerotic plaques where cholesterol and various substances build up, forming atheromas. Normal healthy areas possess a transparent and smooth pink texture. Panel C. Representative image of the aortas from the RBCEV-treated group and control group stained with Oil Red O (ORO) and quantification data of the total lesion, measured as the ORO-positive area per total area of the aortic wall (n=4 mice per group). Panel D. Experimental scheme for the study of RBCEV biodistribution in ApoE−/− mice after 8 weeks on a high-fat diet using RBCEVs labeled with DiR dye (DIR-RBCEVs). Panel E. Representative images of the aorta from mice injected with either DIR-RBCEVs or the DiR dye control and quantification of the DiR signal in the aorta of the two groups. Both the DiR dye solution and DiR-RBCEVs were washed with PBS using several rounds of centrifugation before being injected into the mice. Images were acquired using IVIS (n=3-4 mice). Panel F. Immunofluorescence images of aortic roots from mice treated as described in A. Aortic root sections were stained with antibodies against mouse HO-1 (Green). Panel G. Quantification of HO-1 signals in the plaque area of aortic root sections from F. Pools of 30-36 aortic root sections from 3 different mice in each treatment group were analysed. Student's two-tailed t-test (C, G), *p<0.05.

FIG. 10. Involvement of HO-1 and cholesterol efflux in the reduced lipid accumulation caused by RBCEVs. Panel A. Western blot analysis of HO-1 and GAPDH from macrophages after transfection with the negative control (NC) ASO or HO-1 ASO. Panel B. Flow cytometry analysis of Dil-oxLDL uptake by macrophages after knocking down HO-1 using the HO-1 ASO, relative to the effect of the NC ASO. After transfection with the ASOs, the macrophages were treated with RBCEVs for 4 days, followed by incubation with 10 μg/mL Dil-oxLDL for 24 hours. Data was normalized to the signal in the NC ASO, Dil-oxLDL-treated, RBCEV-nontreated group. Panel C. Cholesterol efflux measured in the supernatant of untreated and RBCEV-treated macrophages, presented as the percentage of the total signal from both the supernatant and the cells. Student's two-tailed t-test (b,c), *p<0.05, ns: not significant (p>0.05).

FIG. 11. Effects of RBCEVs on ApoE knockout mice on a high-fat diet. Panel A. Weight progression of control and RBCEV-treated mice over the course of the treatment. Panel B. Biodistribution of intravenously injected RBCEVs. DiR-labeled RBCEVs and the free DiR dye control were administered via tail vein injection at 50 mg/kg. After 12 hours, the aortas were collected and analyzed using IVIS® Spectrum In Vivo Imaging System.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system (e.g., that is or comprises one or more cells, tissues, organisms, etc.), for example to achieve delivery of an agent that is, is included in, or is otherwise delivered by, the composition.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference”]. In some embodiments, affinity may be assessed relative to a contemporaneous reference; in some embodiments, affinity may be assessed relative to a historical reference. Typically, when affinity is assessed relative to a reference, it is assessed under comparable conditions.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Associated: Two events or entities are “associated” with one another, as that term is used herein, if the presence, level, degree, type and/or form of one is correlated with that of the other. For example, a particular entity (e.g., cargo nucleic acid) is considered to be associated with a biological event (e.g., expression or activity of a polypeptide encoded by a payload nucleic acid, level of cytokine indicative of an inflammatory response, level of expression of a gene regulated by an inflammation-associated regulator, cell viability, etc.), if its presence, level and/or form correlates with incidence and/or intensity of the relevant biological event (e.g., in a cell, tissue or organism, and/or across a relevant population thereof). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell). Binding between two entities may be considered “specific” if, under the conditions assessed, the relevant entities are more likely to associate with one another than with other available binding partners.

Cargo Nucleic Acid: The term “cargo nucleic acid”, as used herein, refers to a nucleic acid that is administered or otherwise delivered to a subject or system of interest (e.g., that is or comprises one or more cells, tissues, organisms, etc). In many embodiments described herein, a cargo nucleic acid is present in and/or delivered from an extracellular vesicle (EV, e.g., a red blood cell extracellular vesicle, RBCEV). In some embodiments, a cargo nucleic acid is or comprises a payload nucleic acid. In some embodiments, a cargo nucleic acid is or comprises a promoting oligonucleotide. In some embodiments, more than one cargo nucleic acid is administered or otherwise delivered to the same subject or system in accordance with the present disclosure. In some embodiments, at least one payload nucleic acid and at least one promoting oligonucleotide are administered or otherwise delivered to the same subject or system in accordance with the present disclosure, in some embodiments as cargo within the same EV (e.g., RBCEV), in some embodiments as separate cargos within different EVs (e.g., RBCEVs) or otherwise separately.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Delivery vehicle: As used herein, the term “delivery vehicle” refers to an agent that complexes or otherwise interacts with nucleic acid for the purpose of delivering said nucleic acid to a system. Delivery vehicles may stabilize nucleic acid in otherwise harsh conditions (e.g., a bloodstream or local tissue environment after in vivo administration). Delivery vehicles may allow for nucleic acid to pass through the plasma membrane of a cell (i.e., be delivered to a cell). Furthermore, delivery vehicles may provide cell-type or tissue-type specificity in delivering of a nucleic acid. Delivery vehicles may be, for example, polyplexes, nanoconjugates, micelles, vesicles, nanocapsules, dendrimers, or nanoparticles (NPs).

Designed: As used herein, the term “designed” refers to an agent (i) whose structure is or was selected by the hand of man; (ii) that is produced by a process requiring the hand of man; and/or (iii) that is distinct from natural substances and other known agents.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. In some embodiments, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, such a manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Homology: As used herein, the term “homology” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially homologous” to one another if their sequences are at least 25%, 30%, 35%, 40%, 45%, 50%, 55%, 60%, 65%, 70%, 75%, 80%, 85%, 90%, 95%, or 99% homologous, meaning that identical or homologous residues are present in corresponding positions of both molecules. Calculation of percent homology of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; residues at corresponding positions are then compared. When a position in the first sequence is occupied by the same residue (e.g., nucleotide or amino acid) as a corresponding position in the second sequence, then the two molecules (i.e., first and second) are identical at that position. When a position in the first sequence is occupied by the same residue or by a structurally and/or functionally related residue (as will be understood by those skilled in the art, in context), then the two molecules are considered “homologous” at that position. Percent homology between two sequences is a function of the number of homologous positions shared by the two sequences being compared, taking into account the number of gaps, and the length of each gap, which needs to be introduced for optimal alignment of the two sequences. Comparison of sequences and determination of percent homology between two sequences can be accomplished using a mathematical algorithm. For example, percent homology between two nucleotide sequences can be determined using the algorithm of Meyers and Miller (CABIOS, 1989, 4:11-17, which is herein incorporated by reference in its entirety), which has been incorporated into the ALIGN program (version 2.0).

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Nanoparticle: As used herein, the term “nanoparticle” refers to a discrete entity of small size, e.g., typically having a longest dimension that is shorter than about 1000 nanometers (nm) and often is shorter than 500 nm, or even 100 nm or less. In many embodiments, a nanoparticle may be characterized by a longest dimension between about 1 nm and about 100 nm, or between about 1 μm and about 500 nm, or between about 1 nm and 1000 nm. In many embodiments, a population of microparticles is characterized by an average size (e.g., longest dimension) that is below about 1000 nm, about 500 nm, about 100 nm, about 50 nm, about 40 nm, about 30 nm, about 20 nm, or about 10 nm and often above about 1 nm. In many embodiments, a microparticle may be substantially spherical (e.g., so that its longest dimension may be its diameter). In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

Nanoparticle composition: As used herein, the term “nanoparticle composition” refers to a composition that contains at least one nanoparticle and at least one additional agent or ingredient. In some embodiments, a nanoparticle composition contains a substantially uniform collection of nanoparticles as described herein.

Nucleic acid: As used herein, the term “nucleic acid” refers to a polymer of at least three nucleotides. In some embodiments, a nucleic acid comprises DNA. In some embodiments comprises RNA. In some embodiments, a nucleic acid is single-stranded. In some embodiments, a nucleic acid is double-stranded. In some embodiments, a nucleic acid comprises both single- and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Payload Nucleic Acid: A “payload nucleic acid” as that term is used herein refers to a nucleic acid that is administered or otherwise delivered to a subject or system of interest (e.g., that is or comprises one or more cells, tissues, organisms, etc.) that results in or is intended to achieve a particular biological result. In many embodiments described herein, a payload nucleic acid encodes an expression product (e.g., a transcript or polypeptide) that achieves or is intended to achieve the relevant result. In some embodiments described herein, a payload nucleic acid wholly or partly makes up a cargo nucleic acid. In many embodiments described herein, a payload nucleic acid is present in and/or delivered from an extracellular vesicle (EV, e.g., a red blood cell extracellular vesicle, RBCEV). In some embodiments, at least one payload nucleic acid and at least one promoting oligonucleotide are administered or otherwise delivered to the same subject or system in accordance with the present disclosure, in some embodiments as cargo within the same EV (e.g., RBCEV), in some embodiments as separate cargos within different EVs (e.g., RBCEVs) or otherwise separately.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces

Promoting Oligonucleotide: As used herein, the term “promoting oligonucleotide” refers to a nucleic acid whose presence is associated with (a) increased level and/or activity of an expression product of a payload; and/or (b) decreased inflammatory and/or otherwise undesirable effect or response (e.g., immune effect or response) associated with administration or delivery of a payload nucleic acid. In some embodiments described herein, a promoting oligonucleotide wholly or partly makes up a cargo nucleic acid. In many embodiments described herein, a promoting oligonucleotide is present in and/or delivered from an extracellular vesicle (EV, e.g., a red blood cell extracellular vesicle, RBCEV). In some embodiments, at least one promoting oligonucleotide and at least one payload nucleic acid are administered or otherwise delivered to the same subject or system in accordance with the present disclosure, in some embodiments as cargo within the same EV (e.g., RBCEV), in some embodiments as separate cargos within different EVs (e.g., RBCEVs) or otherwise separately.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific: As used herein, the term “specific”, with reference to an agent having an activity, is understood by those skilled in the art to mean that the agent discriminates between potential target entities or states. For example, and in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Subject: As used herein, the term “subject” refers to any organism to which a provided composition is or may be administered, e.g., for experimental, diagnostic, prophylactic, cosmetic, and/or therapeutic purposes. Typical subjects include animals (e.g., mammals such as mice, rats, rabbits, non-human primates, and/or humans). In some embodiments, a subject is a human. In some embodiments, a subject is suffering from or susceptible to one or more diseases, disorders, or conditions. In some embodiments, a subject displays one or more symptoms of a disease, disorder, or condition. In some embodiments, a subject has been diagnosed with one or more diseases, disorders, or conditions. In some embodiments, the disease, disorder, or condition is or comprises cancer, or presence of one or more tumors. In some embodiments, the disease, disorder, or condition is or comprises cystic fibrosis. In some embodiments, the subject is receiving or has received certain therapy to diagnose and/or to treat a disease, disorder, or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, reduce the risk of developing the disease, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described herein. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### DETAILED DESCRIPTION OF CERTAIN EMBODIMENTS

The present disclosure, among other things, provides certain technologies relating to treatment of inflammatory diseases, disorders, and conditions.

In some embodiments, the present disclosure provides red blood cell extracellular vesicle (RBCEV) preparations/populations, e.g., formulated for anti-inflammatory effects.

The present disclosure remarkably demonstrates that provided technologies can be useful in treating inflammatory diseases, disorders, and conditions.

In some embodiments, provided technologies can be particularly useful in the context of treating atherosclerosis (e.g., by delivering heme, hemoglobin and/or phosphatidylserine to intraplaque macrophage populations). In some embodiments, provided technologies can be particularly useful for delivery of nucleic acid-loaded RBCEVs with anti-inflammatory effects.

### I. Inflammatory Diseases, Disorders, and Conditions

Technologies provided by the present disclosure achieve effective prevention and/or amelioration of inflammation and may be particularly useful in the treatment of one or more inflammatory diseases, disorders, and conditions.

In some embodiments, an inflammatory disease, disorder, or condition may be associated with physical damage to one or more tissues. In some embodiments, an inflammatory disease, disorder, or condition may be associated with an infection. In some embodiments, an inflammatory disease, disorder, or condition may be associated with autoimmunity. In some embodiments, an inflammatory disease, disorder, or condition may be associated with fibrosis. In some embodiments, an inflammatory disease, disorder, or condition may be associated with one or more genetic mutations. In some embodiments, an inflammatory disease, disorder, or condition may be associated with cancer. In some embodiments, an inflammatory disease, disorder, or condition may be associated with lifestyle and/or environment. In some embodiments, an inflammatory disease, disorder, or condition may be associated with age.

In some embodiments, an inflammatory disease, disorder, or condition is or comprises atherosclerosis.

Atherosclerosis refers to a process where plaque builds up in the interior of arteries, progressively restricting blood flow as the plaques grow in size. These plaques may comprise fatty streaks, fibrosis, calcifications, etc. Significant clinical complications may occur from atherosclerotic plaques occluding an artery to result in stenosis or rupturing to result in thrombosis. In some cases, atherosclerosis can lead to coronary artery disease, stroke, peripheral artery disease, kidney problems, etc. Atherosclerosis has been reviewed, for example, in Insull Jr, W., 2009. The pathology of atherosclerosis: plaque development and plaque responses to medical treatment. The American journal of medicine, 122 (1), pp. S3-S14, incorporated herein in its entirety be reference.

The present disclosure appreciates that certain cell types contribute to the atherosclerotic process. Macrophages are known to amass in atherosclerotic lesions, proliferate locally, ingest lipids, and produce inflammatory signals. See, e.g., Robbins, C. S., et al., 2013. Local proliferation dominates lesional macrophage accumulation in atherosclerosis. Nature medicine, 19 (9), pp. 1166-1172. Macrophages that ingest lipids, particularly low-density lipoprotein (LDL), can form into foam cells, which play a critical role in the occurrence and development of atherosclerosis. See, e.g., Yu, X. H., et al., 2013. Foam cells in atherosclerosis. Clinica chimica acta, 424, pp. 245-252. The present disclosure, among other things, describes compositions and methods (e.g., those comprising certain populations of RBCEVs) that reduce the formation of foam cells.

Among other things, the present disclosure describes populations of RBCEVs that are particularly useful for the treatment of inflammatory diseases, disorders, or conditions (e.g., atherosclerosis).

### II. Extracellular Vesicles

As described herein, an extracellular vesicle (EV) is a lipid-bound vesicle-like structure. In some embodiments, EVs have a membrane. In some embodiments, EVs have a membrane that is a double layer membrane (e.g., a lipid bilayer). In some embodiments, EVs have a membrane that originates from a cell. In some embodiments, EVs have a membrane that originates from the plasma membrane of a cell.

The term extracellular vesicle encompasses exosomes, microvesicles, membrane microparticles, ectosomes, blebs or apoptotic bodies. In some embodiments, an EV is classified as an exosome, microvesicle, membrane microparticle, ectosome, bleb or apoptotic body based on the origin of formation. In preferred aspects, the EVs are RBCEVs. In other words, the EVs are EVs derived from Red Blood Cells.

In some embodiments, EVs are substantially red. In some embodiments, EVs are substantially spherical.

Extracellular vesicles (EVs) have intricate roles in both normal and pathological physiology. They carry signals to distant cells and alter their cellular behaviors. These signals are bioactive compounds, including macromolecules and/or small molecules, which are protected by the lipid bilayer that delineates each vesicle¹. Such protection prolongs the course of action and travel distance of the signaling molecules.

### Populations

In some embodiments, an EV population utilized in accordance with the present disclosure is characterized by an average particle diameter within a range of 50 to 1000 nm. In some embodiments, a relevant EV population is characterized by an average particle diameter within a range of 50 to 750 nm. In some embodiments, a relevant EV population is characterized by an average particle diameter within a range of 50 to 500 nm. In some embodiments, a relevant EV population is characterized by an average particle diameter within a range of 50 to 300 nm. In some embodiments, a relevant EV population is characterized by an average particle diameter within a range of 50 to 200 nm. In some embodiments, a relevant EV population is characterized by an average particle diameter within a range of 50 to 150 nm. In some embodiments, a relevant EV population is characterized by an average particle diameter within a range of 100 to 1000 nm. In some embodiments, a relevant EV population is characterized by an average particle diameter within a range of 100 to 750 nm. In some embodiments, a relevant EV population is characterized by an average particle diameter within a range of 100 to 500 nm. In some embodiments, a relevant EV population is characterized by an average particle diameter within a range of 100 to 300 nm. In some embodiments, a relevant EV population is characterized by an average particle diameter within a range of 100 to 200 nm. In some embodiments, a relevant EV population is characterized by an average particle diameter of at least 100 nm. In some embodiments, a relevant EV population is characterized by an average particle diameter of at most 300 nm.

In some embodiments, EVs within a population of relevant EVs have a particle diameter ranging from 50 to 1000 nm. In some embodiments, EVs within a population of relevant EVs have a particle diameter ranging from 50 to 750 nm. In some embodiments, EVs within a population of relevant EVs have a particle diameter ranging from 50 to 500 nm. In some embodiments, EVs within a population of relevant EVs have a particle diameter ranging from 50 to 300 nm. In some embodiments, EVs within a population of relevant EVs have a particle diameter ranging from 50 to 200 nm. In some embodiments, EVs within a population of relevant EVs have a particle diameter ranging from 50 to 150 nm. In some embodiments, EVs within a population of relevant EVs have a particle diameter ranging from 100 to 1000 nm. In some embodiments, EVs within a population of relevant EVs have a particle diameter ranging from 100 to 750 nm. In some embodiments, EVs within a population of relevant EVs have a particle diameter ranging from 100 to 500 nm. In some embodiments, EVs within a population of relevant EVs have a particle diameter ranging from 100 to 300 nm. In some embodiments, EVs within a population of relevant EVs have a particle diameter ranging from 100 to 200 nm. In some embodiments, EVs within a population of relevant EVs have a particle diameter of at least 100 nm. In some embodiments, EVs within a population of relevant EVs have a particle diameter of at most 300 nm.

A population of EVs (e.g., as may be present in and/or used to manufacture a composition, pharmaceutical composition, medicament, preparation or otherwise) may include EVs with a range of diameters. In some embodiments, the median diameter of EVs within a population is 50, 60, 70, 80, 90, 100, 110, 120, 130, 140, 150, 160, 170, 180, 190, 200, 210, 220, 230, 240, 250, 260, 270, 280, 290, or 300 nm (±1, 2, 3, 4, 5, 6, 7, 8, 9 or 10 nm). In some embodiments, the mean diameter of EVs within a population is 50, 60, 70, 80, 90, 100, 110, 120, 130, 140, 150, 160, 170, 180, 190, 200, 210, 220, 230, 240, 250, 260, 270, 280, 290, or 300 nm (±1, 2, 3, 4, 5, 6, 7, 8, 9 or 10 nm).

A population of EVs may comprise at least 10, 100, 1000, 10⁴, 10⁵, 10⁶, 10⁷, 10⁸, 10⁹, 10¹⁰, 10¹¹, 10¹², 10¹³ or 10¹⁴ EVs. A population of EVs may comprise at least 10, 100, 1000, 10⁴, 10⁵, 10⁶, 10⁷, 10⁸, 10⁹, 10¹⁰, 10¹¹, 10¹², 10¹³ or 10¹⁴ EVs per mL of carrier.

### Red Blood Cell Derived Extracellular Vesicles (RBCEVs)

In some embodiments, EVs are derived from red blood cells. In some embodiments, EVs are red blood cell derived extracellular vesicles (RBCEVs). In some embodiments, EVs are derived from red blood cells ex vivo from a blood draw from a subject.

Red blood cells (e.g, erythrocytes) are enucleated. Red blood cells are characterized in that they do not contain DNA or they contain substantially no DNA. Red blood cells may contain miRNAs or other RNAs. Red blood cells do not contain oncogenic DNA or oncogenic DNA mutations. Red blood cells lack cellular organelles, such as endosomes and endoplasmic reticulum. Red blood cells cannot produce exosomes.

In some embodiments, RBCEVs contain less nucleic acid than EVs that have been derived from other cell types. In some embodiments, RBCEVs do not contain nucleic acid (e.g., DNA) that was present in the cells from which they were derived. In some embodiments, RBCEVs are non-exosomal EVs.

In some embodiments, RBCEVs comprise hemoglobin, stomatin, and/or flotilin-2. In some embodiments, RBCEVs are red in color. In some embodiments, RBCEVs exhibit a domed (i.e., concave) surface, or “cup shape” when viewed under transmission electron microscopes. In some embodiments, RBCEVs comprise cell surface CD235a.

In some embodiments, an RBCEV population is characterized by an average particle diameter within a range of 50 to 1000 nm. In some embodiments, an RBCEV population is characterized by an average particle diameter within a range of 50 to 750 nm. In some embodiments, an RBCEV population is characterized by an average particle diameter within a range of 50 to 500 nm. In some embodiments, an RBCEV population is characterized by an average particle diameter within a range of 50 to 300 nm. In some embodiments, an RBCEV population is characterized by an average particle diameter within a range of 50 to 200 nm. In some embodiments, an RBCEV population is characterized by an average particle diameter within a range of 50 to 150 nm. In some embodiments, an RBCEV population is characterized by an average particle diameter within a range of 100 to 1000 nm. In some embodiments, an RBCEV population is characterized by an average particle diameter within a range of 100 to 750 nm. In some embodiments, an RBCEV population is characterized by an average particle diameter within a range of 100 to 500 nm. In some embodiments, an RBCEV population is characterized by an average particle diameter within a range of 100 to 300 nm. In some embodiments, an RBCEV population is characterized by an average particle diameter within a range of 100 to 200 nm. In some embodiments, an RBCEV population is characterized by an average particle diameter of at least 100 nm. In some embodiments, an RBCEV population is characterized by an average particle diameter of at most 300 nm.

In some embodiments, RBCEVs within a population of RBCEVs have a particle diameter ranging from 50 to 1000 nm. In some embodiments, RBCEVs within a population of RBCEVs have a particle diameter ranging from 50 to 750 nm. In some embodiments, RBCEVs within a population of RBCEVs have a particle diameter ranging from 50 to 500 nm. In some embodiments, RBCEVs within a population of RBCEVs have a particle diameter ranging from 50 to 300 nm. In some embodiments, RBCEVs within a population of RBCEVs have a particle diameter ranging from 50 to 200 nm. In some embodiments, RBCEVs within a population of RBCEVs have a particle diameter ranging from 50 to 150 nm. In some embodiments, RBCEVs within a population of RBCEVs have a particle diameter ranging from 100 to 1000 nm. In some embodiments, RBCEVs within a population of RBCEVs have a particle diameter ranging from 100 to 750 nm. In some embodiments, RBCEVs within a population of RBCEVs have a particle diameter ranging from 100 to 500 nm. In some embodiments, RBCEVs within a population of RBCEVs have a particle diameter ranging from 100 to 300 nm. In some embodiments, RBCEVs within a population of RBCEVs have a particle diameter ranging from 100 to 200 nm. In some embodiments, RBCEVs within a population of RBCEVs have a particle diameter of at least 100 nm. In some embodiments, RBCEVs within a population of RBCEVs have a particle diameter of at most 300 nm.

A population of RBCEVs (e.g., as present in a composition, pharmaceutical composition, medicament, preparation or otherwise) will comprise RBCEVs with a range of diameters. In some embodiments, the median diameter of RBCEVs within a population is 50, 60, 70, 80, 90, 100, 110, 120, 130, 140, 150, 160, 170, 180, 190, 200, 210, 220, 230, 240, 250, 260, 270, 280, 290, or 300 nm (+1, 2, 3, 4, 5, 6, 7, 8, 9 or 10 nm). In some embodiments, the mean diameter of RBCEVs within a population is 50, 60, 70, 80, 90, 100, 110, 120, 130, 140, 150, 160, 170, 180, 190, 200, 210, 220, 230, 240, 250, 260, 270, 280, 290, or 300 nm (±1, 2, 3, 4, 5, 6, 7, 8, 9 or 10 nm).

A population of RBCEVs may comprise at least 10, 100, 1000, 10⁴, 10⁵, 10⁶, 10⁷, 10⁸, 10⁹, 10¹⁰, 10¹¹, 10¹², 10¹³ or 10¹⁴ RBCEVs. A population of RBCEVs may comprise at least 10, 100, 1000, 10⁴, 10⁵, 10⁶, 10⁷, 10⁸, 10⁹, 10¹⁰, 10¹¹, 10¹², 10¹³ or 10¹⁴ RBCEVs per ml of carrier.

In some embodiments, RBCEVs are derived from a human or animal blood sample. In some embodiments, RBCEVs are derived from red blood cells derived from primary cells or immobilized red blood cell lines. In some embodiments, RBCEVs are derived from blood cells type matched to the subject that is to be treated. In some embodiments, RBCEVs are derived from blood cells of Group A, Group B, Group AB, or Group O blood. In some embodiments, RBCEVs are derived from blood cells of Group O blood.

In some embodiments, blood is any blood type. In some embodiments, blood is rhesus positive or rhesus negative. In some embodiments, blood is Group O and/or rhesus negative, such as Type O—. In some embodiments, blood has been determined to be free from disease or disorder. For example, in some embodiments, blood has been determined to be free from HIV, HBV, HCV, syphilis, sickle cell anemia, SARS-COV2, and/or malaria.

In some embodiments, RBCEVs are derived from a blood sample obtained from a subject that is to be treated. In some embodiments, RBCEVs are autologous. In some embodiments, RBCEVs are derived from a blood sample obtained from a subject other than one that is to be treated. In some embodiments, RBCEVs are allogenic.

In some embodiments, RBCEVs are isolated from a sample of red blood cells. Protocols for obtaining EVs from red blood cells are known in the art, for example in Danesh et al. (2014) Blood. 2014 Jan. 30; 123 (5): 687-696. Methods useful for obtaining RBCEVs may include steps of providing or obtaining a sample comprising red blood cells, inducing the red blood cells to produce EVs, and isolating the EVs. A sample may be a whole blood sample. Red blood cells in a sample may be separated from other components of a whole blood sample (e.g., white blood cells or plasma). Red blood cells may be concentrated (e.g., by centrifugation). A blood sample may be subjected to leukocyte reduction.

Cells other than red blood cells may have been removed from the sample, such that the cellular component of the sample is entirely or substantially only red blood cells. In some embodiments, EVs are induced from red blood cells by contacting the cells with a vesicle-inducing agent. In some embodiments, a vesicle-inducing agent is calcium ionophore, lysophosphatidic acid (LPA), or phorbol-12-myristat-13-acetate (PMA). In some embodiments, a vesicle-inducing agent is about 10 nM calcium ionophore.

In some embodiments, RBCEVs are isolated from red blood cells and other components of a sample and/or mixture. In some embodiments, RBCEVs are isolated by centrifugation (with or without ultracentrifugation), precipitation, filtration (e.g., tangential flow filtration), or chromatography.

In some embodiments, red blood cells are separated from a whole blood sample which contains white blood cells and plasma by low-speed centrifugation and leukodepletion filters. In some embodiments, a red blood cell sample comprises no other cell types (e.g., white blood cells). In some embodiments, red blood cells are diluted in buffer (e.g., PBS) prior to contacting with a vesicle-inducing agent. In some embodiments, red blood cells are contacted with a vesicle-inducing agent overnight, or for at least 1, at least 2, at least 3, at least 4, at least 5, at least 6, at least 7, at least 8, at least 9, at least 10, at least 11, at least 12 or more than 12 hours. In some embodiments, red blood cells are contacted with a vesicle-inducing agent at a plurality of time points. In some embodiments, RBCEVs are isolated by subjecting a sample to low-speed centrifugation and/or passing a sample through an about 0.45 μm syringe filter. In some embodiments, RBCEVs are concentrated by ultracentrifugation. In some embodiments, RBCEVs are concentrated by ultracentrifugation at a speed of 10,000×g, 15,000×g, 20,000×g, 25,000×g, 30,000×g, 40,000×g, 50,000×g, 60,000×g, 70,000×g, 80,000×g, 90,000×g or 100,000×g. In some embodiments, RBCEVs are concentrated by ultracentrifugation at a speed within a range of 10,000×g and 50,000×g. In some embodiments, RBCEVs are concentrated by ultracentrifugation at a speed of about 15,000×g. In some embodiments, RBCEVs are concentrated by ultracentrifugation for at least 10 minutes, at least 20 minutes, at least 30 minutes, at least 40 minutes, at least 50 minutes or at least one hour.

In some embodiments, concentrated RBCEVs are suspended in cold PBS. In some embodiments, concentrated RBCEVs are layered on a sucrose cushion. In some embodiments, a sucrose cushion comprises frozen 60% sucrose. In some embodiments, RBCEVs layered on a sucrose cushion are subjected to ultracentrifugation at 100,000×g for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours or longer. In some embodiments, RBCEVs layered on a sucrose cushion are subjected to ultracentrifugation at 100,000×g for about 16 hours. RBCEVs may then be obtained by collecting the red layer above the sucrose cushion.

Methods for isolation and characterization of RBCEVs are described, for example, in Usman et al. (Efficient RNA drug delivery using red blood cell extracellular vesicles. Nature Communications 9, 2359 (2018) doi: 10.1038/s41467-018-04791-8), incorporated herein in its entirety by reference.

EVs originated from red blood cells (RBCEVs) have favorable traits for serving as an effective drug delivery platform. They are devoid of DNA and inherit the allogenic transfusion compatibility from red blood cells, hence providing safe, “off-the-shelf” medication. In addition, red blood cells can be collected from volunteers and induced to release large amounts of RBCEVs by stimulation with calcium ionophore^{3,4}. Therefore, RBCEV production is easily scalable and cost-effective compared with EVs from stem cells or cancer cells. RBCEVs have been demonstrated to deliver antisense oligonucleotide^{3,5,6} peptides⁵, and paclitaxel⁴ for cancer treatment in mouse models. They have also been demonstrated to deliver ASOs for treating acute liver failure⁷. Conjugation of targeting molecules onto RBCEV surface may increase accumulation at select target sites, hence increasing the efficacy of drug delivery^{4,5}.

In RBCEVs, hemoglobin is the most abundant protein. In adults, hemoglobin is mainly present in the HbA1 form, which is composed of two alpha-globin chains and two beta-globin chains. Each globin chain complexes with one heme group to facilitate the transportation of oxygen molecules throughout the body. Hemoglobin is not toxic when contained by RBCs. In hemolytic events, hemoglobin is released from RBCs into blood stream and interstitial space causing toxicity⁸. The toxicity can be neutralized by haptoglobin, a protein secreted from liver cells. Hemoglobin and haptoglobin form a complex that is rapidly processed by macrophages through the CD163 receptor⁹. Upon internalization, hemoglobin is broken down and heme groups are processed by an enzyme called Heme oxygenase 1 (HO-1).

HO-1 has been shown to play a protective role against atherosclerosis^{10,11}. This protective effect is speculated to result from the degradation of heme through a reaction catalyzed by HO-1. In the reaction, heme is broken down into ferrous iron, CO and biliverdin. Biliverdin has antioxidant properties while CO inhibits inflammation^{12,13}. In a mouse model, HO-1-knockdown mice develop an atherosclerosis phenotype with severe aortitis, coronary injuries and fatty streaks¹⁴. In contrast, induced expression of HO-1 suppresses atherosclerosis formation^{15,16}. HO-1 is upregulated in intraplaque non-foamy macrophage populations which are distinct from foam cells¹⁷. These macrophages are identified as Mheme cells, which are formed by intraplaque hemorrhage and are induced by hemoglobin and haptoglobin complexes. Mheme cells were reported to prevent foam cell formation^{18,19}. The present disclosure hypothesizes that hemoglobin is protected in enclosed vesicles of RBCEVs, hence preventing cytotoxicity. In addition, the present disclosure hypothesizes that hemoglobin carried by RBCEVs could exert anti-inflammatory and anti-atherosclerosis effects mediated through HO-1 pathway in macrophages.

### Production

Typically, EVs are produced by budding, and/or shedding off of a parent cell. An extracellular vesicle may be derived from various cell types. In some embodiments, EVs have a similar composition to the cell from which it is derived (e.g., as characterized by the type and/or amount of proteins in the lumen and/or associated with the membrane). In some embodiments, an EV is produced from outward budding and fission of cellular membrane. An EV may be produced via a natural process or a chemically-induced or enhanced process.

In some embodiments, EVs are produced from cells that are contacted with a vesicle-inducing agent. A vesicle-inducing agent may be calcium ionophore, lysophosphatidic acid (LPA), or phorbol-12-myristat-13-acetate (PMA).

In some embodiments, EVs are produced from human cells, or cells of human origin. In some embodiments, EVs are produced from cells that are not modified (e.g., transduced, transfected, infected, or otherwise modified). In some embodiments, EVs are produced from cells that are ex vivo.

In some embodiments, EVs are produced from hematopoietic cells. In some embodiments, EVs are produced from immune cells. For example, EVs may be produced from red blood cells, white blood cells, cancer cells, stem cells, dendritic cells, macrophages, or other cell types.

In some embodiments, EVs are produced from red blood cells which have been isolated from plasma and white blood cells. Red blood cells may be isolated by centrifugation and/or leukodepletion filters. In some embodiments, EVs are produced from red blood cells by contacting the cells with calcium ionophore for a sufficient period of time. In some embodiments, contacting red blood cells with calcium ionophore overnight (e.g., 12 hours) is a sufficient period of time to produce EVs.

In some embodiments, EVs are purified from red blood cells and cellular debris. In some embodiments, EVs are purified from red blood cells and cellular debris by centrifugation. In some embodiments, purified EVs are stored at −80° C.

In some embodiments, an EV is a microvesicle or membrane microparticle produced via chemical induction. In some embodiments, a microvesicle or membrane microparticle is shed from the plasma membrane of a cell and does not originate from the endosomal system.

In some embodiments of the present disclosure, an EV selected for loading with cargo nucleic acid is not an exosome. In some embodiments, an EV selected for loading with cargo nucleic acid is not an ectosome. In some embodiments, an EV selected for loading with cargo nucleic acid is not a bleb. In some embodiments, an EV selected for loading with cargo nucleic acid is not an apoptotic body.

### III. Cargo Nucleic Acids

As described herein, a cargo nucleic acid is a nucleic acid that is administered or otherwise delivered to a subject or system of interest (e.g., that is or comprises one or more cells, tissues, organisms, etc.).

Various aspects of the present disclosure relate to nucleic acid agents (e.g., to cargo nucleic acids such as payload nucleic acids and/or promoting oligonucleotides as described herein). Those skilled in the art will appreciate, reading the present disclosure, that many of its findings are applicable to a variety of different nucleic acid agents (as reviewed, for example, in Roberts, et al., “Advances in oligonucleotide drug delivery.” Nature Reviews Drug Discovery 19.10 (2020) and hereby incorporated by reference in its entirety).

In some embodiments, a nucleic acid agent comprises DNA. In some embodiments, a nucleic acid agent comprises RNA. In some embodiments, a nucleic acid agent is single-stranded. In some embodiments, a nucleic acid agent is double-stranded. In some embodiments, a nucleic acid comprises both single- and double-stranded portions. In some embodiments, a strand of a nucleic acid agent comprises self-complementary element(s) such that one or more double-stranded structures can form by self-hybridization within the strand.

In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”.

In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof), an intercalator (e.g., acridine, psoralen, etc.), or a chelator (e.g., metals, radioactive metals, boron, oxidative metals, etc.). In some embodiments, a non-natural residue comprises one or more modified sugars (e.g., 2′-fluororibose, ribose, 2′-deoxyribose, 2′-amino (2′-NH), 2′-O-methyl (2′-OMe), arabinose, and hexose) as compared to those in natural residues. In some embodiments, a non-natural residue comprises one or more modified bases (e.g., 5-position pyrimidine modifications, 8-position purine modifications, modifications at exocyclic amines, substitution of 4-thiouridine, substitution of 5-bromo- or 5-iodo-uracil, backbone modifications, methylations, unusual base-pairing combinations such as the isobases isocytidine and isoguanidine) as compared to those in natural residues. In some embodiments, a non-natural residue comprises one or more 3′ and 5′ modifications (e.g., capping) as compared to those in natural residues. Further, any of the hydroxyl groups ordinarily present in a sugar may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, or organic capping group moieties of from about 1 to about 20 polyethylene glycol (PEG) polymers or other hydrophilic or hydrophobic biological or synthetic polymers. Nucleic acids may be of variant types, such as locked nucleic acid (LNA), peptide nucleic acid (PNA), or gapmer.

In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Nucleic acid agents, generally, can be super-coiled or not super-coiled. Nucleic acid agents, generally, can be chromosomal or non-chromosomal. Nucleic acid agents may be linear or circular. Nucleic acid agents may be conjugated to, or complexed with, other molecules (e.g., carriers, stabilizers, histones, lipophilic agent, etc.).

In some embodiments, a cargo nucleic acid is present in and/or delivered from a delivery vehicle. In many embodiments described herein, a cargo nucleic acid is present in and/or delivered from an extracellular vesicle (EV, e.g., an RBCEV). In some embodiments, one or more copies of an identical cargo nucleic acid is present in and/or delivered from an extracellular vesicle (EV, e.g., an RBCEV). In some embodiments, two or more non-identical cargo nucleic acids are present in and/or delivered from the same extracellular vesicle (EV, e.g., an RBCEV). One of ordinary skill in the art will appreciate that cargo nucleic acids may be non-identical for a various reasons (e.g., sequence, strandedness; length, chemical composition and/or modification, etc.).

In some embodiments, a cargo nucleic acid is or comprises a payload nucleic acid. In some embodiments, a cargo nucleic acid is or comprises a promoting oligonucleotide. In some embodiments, more than one cargo nucleic acid is administered or otherwise delivered to the same subject or system in accordance with the present disclosure. In some embodiments, at least one payload nucleic acid and at least one promoting oligonucleotide are administered or otherwise delivered to the same subject or system in accordance with the present disclosure, in some embodiments as cargo within the same EV (e.g., RBCEV), in some embodiments as separate cargos within different EVs (e.g., RBCEVs) or otherwise separately.

### Payload Nucleic Acids

As described herein, a payload nucleic acid is a nucleic acid that is administered or otherwise delivered to a subject or system of interest (e.g., that is or comprises one or more cells, tissues, organisms, etc.) that results in or is intended to achieve a particular biological result. In many embodiments described herein, a payload nucleic acid encodes an expression product (e.g., a transcript or polypeptide) that achieves or is intended to achieve the relevant result.

Teachings of the present disclosure relate to payload nucleic acids that are not intended for use with viral vectors. In some embodiments, a payload nucleic acid does not comprise ITR sequences.

In some embodiments, a payload nucleic acid may be delivered to at least one cell type or tissue within a subject or system of interest. In some embodiments, a payload nucleic acid expresses or is intended to express an expression product within the cell type or tissue in which it was delivered. In some embodiments, a payload nucleic acid expresses or is intended to express an expression product which is subsequently secreted and/or released from the cell type or tissue in which it was delivered.

In some embodiments, a payload nucleic acid is therapeutic to a subject or system of interest in which the payload nucleic acid was administered. In some embodiments, a payload nucleic acid is therapeutic to one or more cell types or tissues in which the payload nucleic acid was delivered. In some embodiments, a payload nucleic acid is therapeutic to one or more cell types or tissues other than in which the payload nucleic acid was delivered.

In some embodiments, a payload nucleic acid is or comprises DNA that encodes an expression product.

In some embodiments, a payload nucleic acid that is or comprises DNA has a maximum size of 30,000 kb. A payload nucleic acid that is or comprises DNA may have a size of about 30,000, 25,000, 20,000, 15,000, 10,000, 9,000, 8,000, 7,000, 6,000, 5,000, 4,000, 3,000, 2,000, 1,000 or less kb.

In some embodiments, a payload nucleic acid is or comprises RNA that encodes an expression product.

In some embodiments, a payload nucleic acid that is or comprises RNA has a maximum size of 2,000 kb. A payload nucleic acid that is or comprises RNA may have a size of about 2,000, 1,500, 1,000, 900, 800, 700, 600, 500, 400, 300, 200, 100 or less kb.

In some embodiments, a payload nucleic acid is or comprises a DNA plasmid, an RNA plasmid, a circular DNA, a linear double-stranded DNA, a DNA minicircle, a dumbbell-shaped DNA minimal vector, a doggy bone vector, a closed-end linear DNA vector, a nicked linear DNA vector, an RNA minicircle, a small interfering RNA (siRNA), a messenger RNA (mRNA), a guide RNA (gRNA), a prime editing guide RNA (peg RNA), a CRISPR RNA (crRNA), a trans-activating CRISPR RNA (tracrRNA), a circular RNA, a microRNA (miRNA), a primary miRNA (pri-miRNA), a precursor miRNA (pre-miRNA), a piwi-interacting RNA (piRNA), a transfer RNA (tRNA), a long noncoding RNA (lncRNA), an antisense oligonucleotide (ASO), a short hairpin RNA (shRNA), a small activating RNA (saRNA), a small nucleolar RNAs (snoRNA), a gapmer, a locked nucleic acid (LNA), a peptide nucleic acid (PNA), or an expression vector.

In some embodiments, a payload nucleic acid is or comprises a minicircle. Minicircles are circular replicons around 4 kbp. In some embodiments, a minicircle is or comprises DNA. In some embodiments, a minicircle is or comprises RNA. In some embodiments, a minicircle is double-stranded or comprises double-stranded regions. In some embodiments, a minicircle is synthetically derived. In some embodiments, a minicircle does not comprise an origin of replication and therefore does not replicate within a cell. In some embodiments, a minicircle is or comprises a reporter gene. Minicircles are known to those of ordinary skill in the art (e.g. see Gaspar et al., Expert Opin Biol Ther 15 (3), 2015 incorporated by reference in its entirety herein).

In some embodiments, a payload nucleic acid is or comprises a dumbbell-shaped DNA minimal vector. A dumbbell-shaped DNA minimal vector is or comprises a DNA oligonucleotide with a secondary structure comprising one or more hairpins. Dumbbell-shaped DNA minimal vectors are described, for example, in Yu et al (Nucleic Acids Research 2015: 43 (18): e120), Jiang et al (Molecular Therapy 2016: 24 (9): 1581-1591) and Zanta et al (PNAS 1999:96: 91-96), each incorporated herein by reference in its entirety.

In some embodiments, a payload nucleic acid is or comprises a doggy bone vector. In some embodiments, a payload nucleic acid is or comprises a closed-end linear DNA vector. In some embodiments, a payload nucleic acid is or comprises a nicked linear DNA vector.

In some embodiments, a payload nucleic acid is or comprises a plasmid. In some embodiments, a plasmid is able to replicate independently in a cell. In some embodiments, a plasmid comprises an origin of replication sequence. In some embodiments, a plasmid is a nanoplasmid.

In some embodiments, a payload nucleic acid is or comprises RNA. In some embodiments, a payload nucleic acid is or comprises therapeutic RNA. In some embodiments, a payload nucleic acid is or comprises RNA that encodes an expression product (e.g., one or more polypeptides or antigen-binding molecules). In some embodiments, a payload nucleic acid is or comprises RNA that comprises a sequence complementary to a nucleic acid sequence endogenous to a cell in which the payload nucleic acid is delivered. In some embodiments, a payload nucleic acid is or comprises RNA that is useful in methods of gene silencing or downregulating gene expression.

In some embodiments, a payload nucleic acid is antisense to an endogenous nucleic acid sequence within a cell. In some embodiments, an antisense nucleic acid is single or double-stranded. In some embodiments, an antisense nucleic acid comprises double-stranded RNA (dsRNA) or partially double-stranded RNA that is complementary to a target nucleic acid sequence. In some embodiments, a double-stranded RNA molecule is formed by the complementary pairing between a first RNA portion and a second RNA portion within an antisense nucleic acid. The length of an RNA sequence (i.e. one portion) may be less than 30 nucleotides in length (e.g., 29, 28, 27, 26, 25, 24, 23, 22, 21, 20, 19, 18, 17, 16, 15, 14, 13, 12, 11, 10 or fewer nucleotides). In some embodiments, the length of an RNA sequence is within a range of about 18-24 nucleotides.

In some embodiments, a complementary first RNA portion and a second RNA portion form a “stem” of a hairpin structure. The two portions can be joined by a linking sequence, which may form the “loop” in the hairpin structure. The linking sequence can vary in length and may be, for example, 3, 4, 5, 6, 7, 8, 9, 10, 11, 12, 13, 14, 15, or more nucleotides in length. Suitable linking sequences are known in the art.

In some embodiments, an antisense nucleic acid hybridizes to a corresponding DNA sequence within a cell. An antisense nucleic acid may hybridize to a corresponding mRNA within a cell, forming a double-stranded molecule. An antisense nucleic acid may interfere or otherwise disrupt translation of a complementary mRNA, as translation of double-stranded mRNA does not occur. Antisense inhibition of translation is known in the art (see, e.g., Marcus-Sakura, Anal. Biochem. 1988, 172:289).

In some embodiments, an antisense nucleic acid hybridizes to a corresponding micro RNA (miRNA). In some embodiments, an antisense nucleic acid inhibits the function of a miRNA and/or prevents the miRNA from post-transcriptionally regulating gene expression. In some embodiments, an antisense nucleic acid functions to upregulate expression of one or more genes that are otherwise downregulated by a miRNA. In some embodiments, an antisense nucleic acid functions to downregulate expression of target genes.

Examples of an antisense nucleic acid include, but are not limited to, small interfering RNA (siRNA; including derivatives or pre-cursors, such as nucleotide analogs), short hairpin RNA (shRNA), micro RNA (miRNA), saRNA (small activating RNA), small nucleolar RNA (snoRNA) or derivatives or pre-cursors, long non-coding RNA (lncRNA), or single stranded molecules such as chimeric ASO or gapmers. In some embodiments, an antisense nucleic acid stimulates RNA interference (RNAi) or other cellular degradation mechanisms (e.g., RNase degradation).

In some embodiments, a payload nucleic acid is or comprises a siRNA. A “siRNA,” “small interfering RNA,” “small RNA,” or “RNAi” as provided herein, refers to a nucleic acid that forms a double-stranded RNA, which double-stranded RNA has the ability to reduce or inhibit expression of a gene or target gene when expressed in the same cell as the gene or target gene. Complementary portions of RNA that hybridize to form double-stranded RNA may have substantially or completely complementary sequences. In some embodiments, a siRNA has a sequence that is substantially or completely complementary to a target gene sequence. In some embodiments, a siRNA has a length within a range of about 15-50 nucleotides (e.g., each complementary sequence of double-stranded siRNA is about 15-50 nucleotides in length and the double-stranded siRNA is about 15-50 base pairs in length). A siRNA may have a length within a range of 20-30 nucleotides, 20-25 nucleotides, or 24-29 nucleotides (e.g., 20, 21, 22, 23, 24, 25, 26, 27, 28, 29, or 30 nucleotides in length). RNAi and siRNA are described in, for example, Dana et al., Int J Biomed Sci. 2017; 13 (2): 48-57, herein incorporated by reference in its entirety.

Suitable siRNA molecules for use in the methods of the present invention may be designed by schemes known in the art (see, for example, Elbashire et al., Nature, 2001 411:494-8; Amarzguioui et al., Biochem. Biophys. Res. Commun. 2004 316 (4): 1050-8; and Reynolds et al., Nat. Biotech. 2004, 22 (3): 326-30). In some embodiments, siRNA molecules are designed and/or found from commercial vendors. (e.g., Ambion, Dharmacon, GenScript, Invitrogen OligoEngine, etc.). A potential siRNA candidate may be checked for possible complementation and/or interaction with other nucleic acid sequences or polymorphisms using a BLAST alignment program (see, for example, the National Library of Medicine website). In some embodiments, a number of siRNAs are generated and screened to obtain a potential candidate (see, for example, U.S. Pat. No. 7,078,196). In some embodiments, a siRNA is expressed from a vector and/or produced chemically or synthetically. Synthetic RNAi may be obtained from commercial sources, for example, Invitrogen (Carlsbad, California). RNAi vectors may be obtained from commercial sources, for example, Invitrogen.

In some embodiments, a payload nucleic acid is or comprises a miRNA. The term “miRNA” is used in accordance with its ordinary meaning and refers to a small non-coding RNA molecule capable of post-transcriptionally regulating gene expression. In some embodiments, a miRNA is a nucleic acid that has substantial or complete identity to a target gene. In some embodiments, a miRNA inhibits gene expression by interacting with a complementary cellular mRNA thereby interfering with the expression of the complementary mRNA. In some embodiments, a miRNA has a length within a range of about 15-50 nucleotides. (e.g., each complementary sequence of miRNA is about 15-50 nucleotides in length and double-stranded miRNA is about 15-50 base pairs in length). In some embodiments, a miRNA comprises a stem-loop and/or hairpin structure. In some embodiments, a miRNA is synthetic or recombinant. In some embodiments, a miRNA is associated with cancer. In some embodiments, a miRNA is miR-125b.

In some embodiments, a payload nucleic acid is or comprises an expression vector or expression cassette sequence. The terms expression vector or expression cassette sequence refer to a nucleic acid molecule used to express exogenous nucleic acid within a cell. Suitable expression vectors and expression cassettes are known in the art. Expression vectors may comprise elements that facilitate the expression of one or more nucleic acid sequences in a target system (e.g. cell, tissue, organism, etc.).

In some embodiments, an expression vector comprises a promoter sequence operably linked to the nucleotide sequence encoding the nucleic acid sequence to be expressed. In some embodiments, an expression vector comprises a termination codon. In some embodiments, an expression vector comprises expression enhancers. Suitable promoters, termination codons, and enhancers may be used and are known in the art.

In some embodiments, a payload nucleic acid is or comprises a plurality of expression vectors encoding for different peptides or proteins. The different peptides or proteins may be interrelated, such as subunits or components of the same molecule, or molecules that have an interlinked operation, such as components of the same biological pathways, or exhibit a ligand: receptor binding relationship.

In some embodiments, a payload nucleic acid is or comprises a first expression vector encoding a first protein of a protein complex and a further expression vector encoding a further protein of the protein complex. The further protein may be non-identical to the first protein. In some embodiments, a payload nucleic acid is or comprises a first expression vector encoding a first domain of a protein and a further expression vector encoding a further domain of the protein. In some embodiments, a payload nucleic acid is or comprises a first expression vector encoding a first segment of a protein and a further expression vector encoding a further segment of the protein.

In some embodiments, a payload nucleic acid expresses or is intended to express an expression product that is endogenous to the subject or system of interest in which the payload nucleic acid is administered. In some embodiments, a payload nucleic expresses or is intended to express a functional gene, or fragment thereof, to replace and/or supplement a gene that is otherwise not fully functional.

In some embodiments, a payload nucleic acid expresses or is intended to express an expression product that is useful in treating a neurological disease, disorder or condition.

In some embodiments, a payload nucleic acid expresses or is intended to express an expression product that is useful in treating an inflammatory disease, disorder or condition.

In some embodiments, a neurological disease, disorder or condition is or comprises Alzheimer's Disease. In some embodiments, a neurological disease, disorder or condition is or comprises Parkinson's Disease.

In some embodiments, a payload nucleic acid expresses or is intended to express an expression product that is exogenous to the subject or system of interest in which the payload nucleic acid is administered. In some embodiments, a payload nucleic acid is or comprises a transgene.

In some embodiments, a payload nucleic acid encodes and/or expresses (or is the complement of a nucleic acid that encodes or expresses) an antibody, an antibody gene therapy system, and/or an antigen-binding molecule.

An antibody gene therapy system refers to a system in which nucleic acids encoding an antibody of interest are delivered to cells wherein said cells produce and secrete the encoded antibody. In some embodiments, a payload nucleic acid encodes and/or expresses (or is the complement of a nucleic acid that encodes or expresses) one or more components of an antibody gene therapy system. In some embodiments, an antibody gene therapy system is encoded by the same nucleic acid molecule or separate nucleic acid molecules. In some embodiments, an antibody gene therapy system is encoded by one or more DNA molecules. In some embodiments, an antibody gene therapy system is encoded by one or more plasmids. In some embodiments, an antibody gene therapy system is encoded by one or more expression vectors. In some embodiments, an antibody gene therapy system is encoded by one or more mRNA molecules. In some embodiments, an antibody gene therapy system is encoded by one or more minicircles. In some embodiments, an antibody gene therapy system is encoded by one or more dumbbell-shaped DNA minimal vectors.

An antigen-binding molecule refers to a molecule which is capable of binding to a target antigen. An antigen-binding molecule may be a monoclonal antibody, a polyclonal antibody, a monospecific antibody, a multispecific antibody (e.g., a bispecific antibody), or an antibody fragment (e.g., Fv, scFv, Fab, scFab, F(ab′)2, Fab2, diabody, triabody, scFv-Fc, minibody, single domain antibody (e.g., VhH), etc.), as long as it displays binding to the relevant target molecule(s).

In some embodiments, an antibody, or fragment thereof, or antigen-binding molecule is human, humanized, murine, camelid, chimeric, or from another suitable source. In some embodiments, an antibody, or fragment thereof, or antigen-binding molecule is humanized. Methods of humanizing antibodies may involve the fusing of variable domains of rodent origin to constant domains of human origin such that the resultant antibody retains the antigenic specificity of the rodent parented antibody, for example, as described in Morrison et al (1984) Proc. Natl. Acad. Sd. USA 81, 6851-6855.

Monoclonal antibodies (mAbs) refer to a homogenous population of antibodies that specifically bind a single epitope on an antigen. Suitable monoclonal antibodies to selected antigens may be prepared by known techniques, for example, those disclosed in Köhler, G.; Milstein, C. (1975) “Continuous cultures of fused cells secreting antibody of predefined specificity”. Nature 256 (5517): 495; Siegel D L (2002). “Recombinant monoclonal antibody technology”. Schmitz U, Versmold A, Kaufmann P, Frank H G (2000) “Phage display: a molecular tool for the generation of antibodies—a review”. Placenta. 21 Suppl A: S106-12; Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990) “Phage antibodies: filamentous phage displaying antibody variable domains” Nature 348 (6301): 552-554; “Monoclonal Antibodies: A manual of techniques”; H Zola (CRC Press, 1988); and “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799)).

Polyclonal antibodies (pAbs) refer to a heterologous population of antibodies that bind different epitopes on a single antigen. In some embodiments, polyclonal antibodies are monospecific. Suitable polyclonal antibodies can be prepared using methods known in the art.

In some embodiments, a payload nucleic acid encodes and/or expresses (or is the complement of a nucleic acid that encodes or expresses) a heavy chain or light chain of an antibody. In some embodiments, a payload nucleic acid encodes and/or expresses (or is the complement of a nucleic acid that encodes or expresses) a heavy chain of an antibody, and a further payload nucleic acid encodes and/or expresses (or is the complement of a nucleic acid that encodes or expresses) a light chain of an antibody, and when the at least two payloads are delivered in the same cell, cell type, or tissue an antibody is formed.

An antibody fragment refers to a fragment or shortened sequence of an antibody which retains binding to relevant target molecule(s). Antigenic specificity is conferred by variable domains and is independent of constant domains. Molecules that possess antigen-binding properties include, but are not limited to, Fab-like molecules (Better et al. (1988) Science 240, 1041); Fv molecules (Skerra et al. (1988) Science 240, 1038); single-chain Fv (ScFv) molecules where the VH and VL partner domains are linked via a flexible oligopeptide (Bird et al. (1988) Science 242, 423; Huston et al. (1988) Proc. Natl. Acad. Sd. USA 85, 5879) and single domain antibodies (dAbs) comprising isolated V domains (Ward et al. (1989) Nature 341, 544). A general review of the techniques involved in synthesizing antibody fragments which retain antigenic specificity can be found in Winter & Milstein (1991) Nature 349, 293-299.

A single-chain variable fragment (scFv) refers to molecules wherein the heavy chain variable domain (VH) and light chain variable domain (VL) are covalently linked (e.g., by a peptide or a flexible oligopeptide). A single domain antibody (sdAb) refers to molecules comprising one, two, or more single monomeric variable antibody domains. A single chain antibody (scAb) refers to molecules comprising covalently linked VH and VL partner domains (e.g., by a peptide or a flexible oligopeptide).

A payload nucleic acid may encode and/or express (or is the complement of a nucleic acid that encodes or expresses) 3F8, 8H9, Abagovomab, Abciximab (ReoPro), Abituzumab, Abrezekimab, Abrilumab, Actoxumab, Adalimumab (Humira), Adecatumumab, Aducanumab, Afasevikumab, Afelimomab, Alacizumab pegol, Alemtuzumab (Lemtrada), Alirocumab (Praluent), Altumomab pentetate (Hybri-ceaker), Amatuximab, Amivantamab, Anatumomab mafenatox, Andecaliximab, Anetumab ravtansine, Anifrolumab, Ansuvimab (Ebanga), Anrukinzumab (=IMA-638), Apolizumab, Aprutumab ixadotin, Arcitumomab (CEA-Scan), Ascrinvacumab, Aselizumab, Atezolizumab (Tecentriq), Atidortoxumab, Atinumab, Atoltivimab, Atoltivimab/maftivimab/odesivimab (Inmazeb), Atorolimumab, Avelumab (Bavencio), Azintuxizumab vedotin, Balstilimab, Bamlanivimab, Bapineuzumab, Basiliximab (Simulect), Bavituximab, BCD-100, Bectumomab (LymphoScan), Begelomab, Belantamab mafodotin (Blenrep), Belimumab (Benlysta), Bemarituzumab, Benralizumab (Fasenra), Berlimatoxumab, Bermekimab (Xilonix), Bersanlimab, Bertilimumab, Besilesomab (Scintimun), Bevacizumab (Avastin), Bezlotoxumab (Zinplava), Biciromab (FibriScint), Bimagrumab, Bimekizumab, Birtamimab, Bivatuzumab, Bleselumab, Blinatumomab (Blincyto), Blontuvetmab (Blontress), Blosozumab, Bococizumab, Brazikumab, Brentuximab vedotin (Adcentris), Briakinumab, Brodalumab (Siliq), Brolucizumab (Beovu), Brontictuzumab, Burosumab (Crysvita), Cabiralizumab, Camidanlumab tesirine, Camrelizumab, Canakinumab (llaris), Cantuzumab mertansine, Cantuzumab ravtansine, Caplacizumab (Cablivi), Casirivimab, Capromab (Prostascint), Carlumab, Carotuximab, Catumaxomab (Removab), cBR96-doxorubicin immunoconjugate, Cedelizumab, Cemiplimab (Libtayo), Cergutuzumab amunaleukin, Certolizumab pegol (Cimzia), Cetrelimab, Cetuximab (Erbitux), Cibisatamab, Cirmtuzumab, Citatuzumab bogatox, Cixutumumab, Clazakizumab, Clenoliximab, Clivatuzumab tetraxetan (hPAM4-Cide), Codrituzumab, Cofetuzumab pelidotin, Coltuximab ravtansine, Conatumumab, Concizumab, Cosfroviximab (ZMapp), Crenezumab, Crizanlizumab (Adakveo), Crotedumab, CR6261, Cusatuzumab, Dacetuzumab, Daclizumab (Zenapax), Dalotuzumab, Dapirolizumab pegol, Daratumumab (Darzalex), Dectrekumab, Demcizumab, Denintuzumab mafodotin, Denosumab (Prolia), Depatuxizumab mafodotin, Derlotuximab biotin, Detumomab, Dezamizumab, Dinutuximab (Unituxin), Dinutuximab beta (Qarziba), Diridavumab, Domagrozumab, Dorlimomab aritox, Dostarlimab, Drozitumab, DS-8201, Duligotuzumab, Dupilumab (Dupixent), Durvalumab (Imfinzi), Dusigitumab, Duvortuxizumab, Ecromeximab, Eculizumab (Soliris), Edobacomab, Edrecolomab (Panorex), Efalizumab (Raptiva), Efungumab (Mycograb), Eldelumab, Elezanumab, Elgemtumab, Elotuzumab (Empliciti), Elsilimomab, Emactuzumab, Emapalumab (Gamifant), Emibetuzumab, Emicizumab (Hemlibra), Enapotamab vedotin, Enavatuzumab, Enfortumab vedotin (Padcev), Enlimomab pegol, Enoblituzumab, Enokizumab, Enoticumab, Ensituximab, Epcoritamab, Epitumomab cituxetan, Epratuzumab, Eptinezumab (Vyepti), Erenumab (Aimovig), Erlizumab, Ertumaxomab (Rexomun), Etaracizumab (Abegrin), Etesevimab, Etigilimab, Etrolizumab, Evinacumab (Evkeeza), Evolocumab (Repatha), Exbivirumab, Fanolesomab (NeutroSpec), Faralimomab, Faricimab, Farletuzumab, Fasinumab, FBTA05 (Lymphomun), Felvizumab, Fezakinumab, Fibatuzumab, Ficlatuzumab, Figitumumab, Firivumab, Flanvotumab, Fletikumab, Flotetuzumab, Fontolizumab (HuZAF), Foralumab, Foravirumab, Fremanezumab (Ajovy), Fresolimumab, Frovocimab, Frunevetmab, Fulranumab, Futuximab, Galcanezumab (Emgality), Galiximab, Gancotamab, Ganitumab, Gantenerumab, Gatipotuzumab, Gavilimomab, Gedivumab, Gemtuzumab ozogamicin (Mylotarg), Gevokizumab, Gilvetmab, Gimsilumab, Girentuximab (Rencarex), Glembatumumab vedotin, Golimumab (Simponi), Gomiliximab, Gosuranemab, Guselkumab (Tremfya), lanalumab, Ibalizumab (Trogarzo), IBI308, Ibritumomab tiuxetan (Zevalin), Icrucumab, Idarucizumab (Praxbind), Ifabotuzumab, Igovomab (Indimacis-125), Iladatuzumab vedotin, IMAB362, Imalumab, Imaprelimab, Imciromab (Myoscint), Imdevimab, Imgatuzumab, Inclacumab, Indatuximab ravtansine, Indusatumab vedotin, Inebilizumab (Uplizna), Infliximab (Remicade), Intetumumab, Inolimomab, Inotuzumab ozogamicin (Besponsa), Ipilimumab (Yervoy), lomab-B, Iratumumab, Isatuximab (Sarclisa), Iscalimab, Istiratumab, Itolizumab (Alzumab), Ixekizumab (Taltz), Keliximab, Labetuzumab (CEA-Cide), Lacnotuzumab, Ladiratuzumab vedotin, Lampalizumab, Lanadelumab (Takhzyro), Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab (Cytopoint), Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab (Margenza), Marstacimab, Maslimomab, Mavrilimumab, Matuzumab, Mepolizumab (Bosatria), Metelimumab, Milatuzumab, Minretumomab, Mirikizumab, Mirvetuximab soravtansine, Mitumomab, Modotuximab, Mogamulizumab (Poteligeo), Monalizumab, Morolimumab, Mosunetuzumab, Motavizumab (Numax), Moxetumomab pasudotox (Lumoxiti), Muromonab-CD3 (Orthoclone OKT3), Nacolomab tafenatox, Namilumab, Naptumomab estafenatox, Naratuximab emtansine, Narnatumab, Narsoplimab, Natalizumab (Tysabri), Navicixizumab, Navivumab, Naxitamab (Danyelza), Nebacumab, Necitumumab (Portrazza), Nemolizumab, NEOD001, Nerelimomab, Nesvacumab, Netakimab (Efleira), Nimotuzumab (BioMab-EGFR, Theracim, Theraloc), Nirsevimab, Nivolumab (Opdivo), Nofetumomab merpentan (Verluma), Obiltoxaximab (Anthim), Obinutuzumab (Gazyva), Ocaratuzumab, Ocrelizumab (Ocrevus), Odesivimab, Odulimomab, Ofatumumab (Arzerra, Kesimpta), Olaratumab (Lartruvo), Oleclumab, Olendalizumab, Olokizumab, Omalizumab (Xolair), Omburtamab, OMS721, Onartuzumab, Ontuxizumab, Onvatilimab, Opicinumab, Oportuzumab monatox (Vicinium), Oregovomab (OvaRex), Orticumab, Otelixizumab, Otilimab, Otlertuzumab, Oxelumab, Ozanezumab, Ozoralizumab, Pagibaximab, Palivizumab (Synagis, Abbosynagis), Pamrevlumab, Panitumumab (Vectibix), Pankomab, Panobacumab, Parsatuzumab, Pascolizumab, Pasotuxizumab, Pateclizumab, Patritumab, PDR001, Pembrolizumab (Keytruda), Pemtumomab (Theragyn), Perakizumab, Pertuzumab (Perjeta), Pexelizumab, Pidilizumab, Pinatuzumab vedotin, Pintumomab, Placulumab, Prezalumab, Plozalizumab, Pogalizumab, Polatuzumab vedotin (Polivy), Ponezumab, Porgaviximab, Prasinezumab, Prezalizumab, Priliximab, Pritoxaximab, Pritumumab, PRO 140, Quilizumab, Racotumomab (Vaxira), Radretumab, Rafivirumab, Ralpancizumab, Ramucirumab (Cyramza), Ranevetmab, Ranibizumab (Lucentis), Raxibacumab, Ravagalimab, Ravulizumab (Ultomiris), Refanezumab, Regavirumab, Regdanvimab, REGN-EB3, Relatlimab, Remtolumab, Reslizumab (Cinqair), Retifanlimab, Rilotumumab, Rinucumab, Risankizumab (Skyrizi), Rituximab (MabThera, Rituxan), Rivabazumab pegol, Robatumumab, Rmab (RabiShield), Roledumab, Romilkimab, Romosozumab (Evenity), Rontalizumab, Rosmantuzumab, Rovalpituzumab tesirine, Rovelizumab (LeukArrest), Rozanolixizumab, Ruplizumab (Antova), SA237, Sacituzumab govitecan (Trodelvy), Samalizumab, Samrotamab vedotin, Sarilumab (Kevzara), Satralizumab (Enspryng), Satumomab pendetide, Secukinumab (Cosentyx), Selicrelumab, Seribantumab, Setoxaximab, Setrusumab, Sevirumab, Sibrotuzumab, SGN-CD19A, SHP647, Sifalimumab, Siltuximab (Sylvant), Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab (LeukoScan), Suptavumab, Sutimlimab, Suvizumab, Suvratoxumab, Tabalumab, Tacatuzumab tetraxetan (AFP-Cide), Tadocizumab, Tafasitamab (Monjuvi), Talacotuzumab, Talizumab, Talquetamab, Tamtuvetmab (Tactress), Tanezumab, Taplitumomab paptox, Tarextumab, Tavolimab, Teclistamab, Tefibazumab (Aurexis), Telimomab aritox, Telisotuzumab, Telisotuzumab vedotin, Tenatumomab, Teneliximab, Teplizumab, Tepoditamab, Teprotumumab (Tepezza), Tesidolumab, Tetulomab, Tezepelumab, TGN1412, Tibulizumab, Tildrakizumab (llumya), Tigatuzumab, Timigutuzumab, Timolumab, Tiragolumab, Tiragotumab, Tislelizumab, Tisotumab vedotin, TNX-650, Tocilizumab (Actemra, RoActemra), Tomuzotuximab, Toralizumab, Toripalimab (Tuoyi), Tosatoxumab, Tositumomab (Bexxar), Tovetumab, Tralokinumab, Trastuzumab (Herceptin), [fam]-trastuzumab deruxtecan (Enhertu), Trastuzumab duocarmazine (Kadcyla), Trastuzumab emtansine (Kadcyla), TRBS07 (Ektomab), Tregalizumab, Tremelimumab, Trevogrumab, Tucotuzumab celmoleukin, Tuvirumab, Ublituximab, Ulocuplumab, Urelumab, Urtoxazumab, Ustekinumab (Stelara), Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab (Entyvio), Veltuzumab, Vepalimomab, Vesencumab, Visilizumab (Nuvion), Vobarilizumab, Volociximab, Vonlerolizumab, Vopratelimab, Vorsetuzumab mafodotin, Votumumab (HumaSPECT), Vunakizumab, Xentuzumab, XMAB-5574, Zalutumumab, Zanolimumab, Zatuximab, Zenocutuzumab, Ziralimumab, Zolbetuximab (IMAB362, Claudiximab), or Zolimomab aritox. An antigen-binding molecule may be a derivative of any of the abovementioned antibodies.

In some embodiments, a payload nucleic acid encodes and/or expresses (or is the complement of a nucleic acid that encodes or expresses) one or more components of a gene editing system.

CRISPR is an abbreviation of Clustered Regularly Interspaced Short Palindromic Repeats. CRISPR comprises segments of DNA containing short, repetitive base sequences in a palindromic repeat (wherein the sequence of nucleotides is the same in both directions). Each repetition is followed by short segments of spacer DNA from previous integration of foreign DNA from a virus or plasmid. Small clusters of Cas (CRISPR-associated) genes are located next to CRISPR sequences. RNA harboring the spacer sequence helps Cas (CRISPR-associated) proteins recognize and cut foreign pathogenic DNA. Other RNA-guided Cas proteins cut foreign RNA. An embodiment of the CRISPR/Cas system, CRISPR/Cas9, has been modified to edit genomes. By delivering the Cas9 nuclease and a synthetic guide RNA (gRNA) into a cell, the cell's genome can be cut at a desired location, allowing existing genes to be removed and/or new ones added. CRISPR/Cas systems fall into two classes. Class 1 systems use a complex of multiple Cas proteins to degrade foreign nucleic acids. Class 2 systems use a single large Cas protein for the same purpose. Class 1 is divided into types I, III, and IV; class 2 is divided into types II, V, and VI. CRISPR genome editing uses a type II CRISPR system.

In some embodiments, a payload nucleic acid encodes and/or expresses (or is the complement of a nucleic acid that encodes or expresses) one or more components of a CRISPR/Cas gene editing system. In some embodiments, a payload nucleic acid recognizes a particular target sequence. In some embodiments, a payload nucleic acid is or comprises a guide RNA (gRNA). In some embodiments, a guide RNA comprises a CRISPR RNA (crRNA) and a trans-activating CRISPR RNA (tracrRNA). crRNA may comprise a sequence that binds and/or identifies a host DNA sequence and a region that binds to tracrRNA to form an active complex. In some embodiments, a gRNA combines both crRNA and tracrRNA thereby encoding an active complex. In some embodiments, a gRNA may comprises multiple crRNAs and/or multiple tracrRNAs. In some embodiments, a gRNA is designed to bind and/or otherwise identify a sequence or gene of interest. In some embodiments, a gRNA targets a sequence or gene of interest for cleavage. In some embodiments, a template DNA sequence is included. In some embodiments, a template DNA sequence is utilized in either non-homologous end joining (NHEJ) or homology directed repair (HDR).

In some embodiments, a payload nucleic acid encodes and/or expresses (or is the complement of a nucleic acid that encodes or expresses) a nuclease. In some embodiments, a payload nucleic acid encodes and/or expresses (or is the complement of a nucleic acid that encodes or expresses) a Cas nuclease. One of ordinary skill in the art will appreciate that Cas nuclease may refer to any Cas protein (e.g., Cas 9, Cas12, etc.). One of ordinary skill in the art will appreciate that a nuclease may refer to any protein that functions to modify nucleic acid (e.g., single strand nicking, double strand breaking, DNA binding, etc.). A nuclease recognizes a DNA site and allows for site-specific DNA editing. In some embodiments, a nuclease is modified. In some embodiments, a nuclease is fused to a reverse transcriptase. In some embodiments, a nuclease is catalytically inactive. In some embodiments, a nuclease is fused to a transcription factor. A modified nuclease may be useful, for example, in a prime editing system or in systems to regulate transcription.

In some embodiments, a payload nucleic acid encodes and/or expresses (or is the complement of a nucleic acid that encodes or expresses) at least a gRNA and a nuclease. In some embodiments, a payload nucleic acid encodes and/or expresses (or is the complement of a nucleic acid that encodes or expresses) at least a gRNA and a nuclease on a plasmid. In some embodiments, a gRNA and a nuclease are encoded on a single plasmid. In some embodiments, a gRNA and a nuclease are encoded on separate plasmids.

In some embodiments, a payload nucleic acid is or comprises a DNA repair template. In some embodiments, a DNA repair template is or comprises a linear double-stranded DNA. In some embodiments, a DNA repair template is a plasmid. In some embodiments, a DNA repair template is present on the same nucleic acid which encodes a gRNA and/or nuclease. In some embodiments, a DNA repair template is present on a separate nucleic acid from the nucleic acid which encodes a gRNA and/or a nuclease.

CRISPR/Cas9 and related systems (e.g., CRISPR/Cpf1, CRISPR/C2c1, CRISPR/C2c2 and CRISPR/C2c3) are reviewed, for example, in Nakade et al., Bioengineered (2017) 8 (3): 265-273, which is hereby incorporated by reference in its entirety. These systems comprise an endonuclease (e.g., Cas9, Cpf1, etc.) and a single-guide RNA (sgRNA) molecule. A sgRNA can be engineered to target endonuclease activity to nucleic acid sequences of interest.

In some embodiments, a payload nucleic acid encodes and/or expresses (or is the complement of a nucleic acid that encodes or expresses) one or more components of a gene editing system other than a CRISPR/Cas gene editing system (e.g., zinc finger nucleases (ZFNs) or transcription activator-like effector nucleases (TALENs)).

In some embodiments, a gene editing system specifically targets a miRNA. In some embodiments, a gene editing system specifically targets miR-125b.

In some embodiments, a gene editing system employs targeted gene editing using a site-specific nuclease (SSN). Gene editing with SSNs is reviewed, for example, in Eid and Mahfouz, Exp Mol Med. 2016 October; 48 (10): e265, which is hereby incorporated by reference in its entirety. Enzymes capable of creating site-specific double strand breaks (DSBs) may be engineered to introduce DSBs to target nucleic acid sequence(s) of interest. DSBs may be repaired by error-prone non-homologous end-joining (NHEJ), in which the two ends of the break are rejoined, often with insertion or deletion of nucleotides. Alternatively, DSBs may be repaired by homology-directed repair (HDR), in which a DNA template with ends homologous to the break site is supplied and introduced at the site of the DSB.

SSNs capable of being engineered to generate target nucleic acid sequence-specific DSBs include ZFNs, TALENs and clustered regularly interspaced palindromic repeats/CRISPR-associated-9 (CRISPR/Cas9) systems.

ZFN systems are reviewed, for example, in Umov et al., Nat Rev Genet. (2010) 11 (9): 636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleic acid sequence.

ZFNs work in pairs as the endonuclease (e.g., FokI) functions as a dimer. A ZFN system comprises two monomers with unique DNA recognition sites in the target genome with proper orientation (i.e. on opposite DNA strands) and spacing to allow the endonuclease to function.

In some embodiments, a payload nucleic acid encodes and/or expresses (or is the complement of a nucleic acid that encodes or expresses) one or more components of a ZFN gene editing system. In some embodiments, a ZFN gene editing system comprises a ZFN pair having two polypeptide monomers. In some embodiments, a ZFN gene editing system is encoded by the same nucleic acid molecule or separate nucleic acid molecules. In some embodiments, a ZFN gene editing system is encoded by one or more DNA molecules. In some embodiments, a ZFN gene editing system is encoded by one or more plasmids. In some embodiments, a ZFN gene editing system is encoded by one or more expression vectors. In some embodiments, a ZFN gene editing system is encoded by one or more mRNA molecules. In some embodiments, a ZFN gene editing system is encoded by one or more minicircles. In some embodiments, a ZFN gene editing system is encoded by one or more dumbbell-shaped DNA minimal vectors.

In some embodiments, two payload nucleic acids comprise a first nucleic acid molecule that encodes first monomer of a ZFN pair and a further nucleic acid molecule that encodes a second monomer of a ZFN pair. The nucleic acids may comprise an expression cassette such that the ZFN monomers are expressed within a target cell. The expressed ZFN monomers may bind to their respective DNA recognition sites and allow dimerization of endonuclease. The endonuclease may function to introduce a DSB into the DNA.

TALEN systems are reviewed, for example, in Mahfouz et al., Plant Biotechnol J. (2014) 12 (8): 1006-14, which is hereby incorporated by reference in its entirety. TALENs comprise a programmable DNA-binding TALE domain and a DNA-cleaving domain (e.g., a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: “HD” binds to C, “NI” binds to A, “NG” binds to T and “NN” or “NK” binds to G (see, for example, Moscou and Bogdanove, Science (2009) 326 (5959): 1501 which is hereby incorporated by reference in its entirety).

TALENs work in pairs as the endonuclease (e.g., FokI) functions as a dimer. A TALEN system comprises two monomers with unique DNA recognition sites in the target genome with proper orientation (i.e., on opposite DNA strands) and spacing to allow the endonuclease to function.

In some embodiments, a payload nucleic acid encodes and/or expresses (or is the complement of a nucleic acid that encodes or expresses) one or more components of a TALEN gene editing system. In some embodiments, a TALEN gene editing system comprises a TALEN pair having two polypeptide monomers. In some embodiments, a TALEN gene editing system is encoded by the same nucleic acid molecule or separate nucleic acid molecules. In some embodiments, a TALEN gene editing system is encoded by one or more DNA molecules. In some embodiments, a TALEN gene editing system is encoded by one or more plasmids. In some embodiments, a TALEN gene editing system is encoded by one or more expression vectors. In some embodiments, a TALEN gene editing system is encoded by one or more mRNA molecules. In some embodiments, a TALEN gene editing system is encoded by one or more minicircles. In some embodiments, a TALEN gene editing system is encoded by one or more dumbbell-shaped DNA minimal vectors.

In some embodiments, two payload nucleic acids comprise a first nucleic acid molecule that encodes first monomer of a TALEN pair and a further nucleic acid molecule that encodes a second monomer of a TALEN pair. The nucleic acids may comprise an expression cassette such that the TALEN monomers are expressed within a target cell. The expressed ZFN monomers may bind to their respective DNA recognition sites and allow dimerization of endonuclease. The endonuclease may function to introduce a DSB into the DNA.

In some embodiments, a payload nucleic acid encodes and/or expresses (or is the complement of a nucleic acid that encodes or expresses) a vaccine. In some embodiments, a payload nucleic acid encodes and/or expresses (or is the complement of a nucleic acid that encodes or expresses) an epitope sequence.

In some embodiments, a payload nucleic acid encodes and/or expresses (or is the complement of a nucleic acid that encodes or expresses) a vaccine to cancer. Cancer vaccines involve displaying a tumor-specific antigen or a tumor-associated antigen to a subject's immune system such that the immune system is able to more effectively recognize cancerous cells. Cancer vaccines are reviewed, for example, in Vergati, Matteo, et al. “Strategies for cancer vaccine development.” Journal of Biomedicine and Biotechnology (2010), which is hereby incorporated by reference. One of ordinary skill in the art will be able to select a tumor-specific antigen or tumor-associated antigen for any particular cancer type using methods known in the art. In some embodiments, a payload nucleic acid encodes and/or expresses (or is the complement of a nucleic acid that encodes or expresses) a tumor-specific antigen. In some embodiments, a payload nucleic acid encodes and/or expresses (or is the complement of a nucleic acid that encodes or expresses) a tumor-associated antigen.

In some embodiments, a cancer vaccine is encoded by one or more DNA molecules. In some embodiments, a cancer vaccine is encoded by one or more plasmids. In some embodiments, a cancer vaccine is encoded by one or more expression vectors. In some embodiments, a cancer vaccine is encoded by one or more mRNA molecules. In some embodiments, a cancer vaccine is encoded by one or more minicircles. In some embodiments, a cancer vaccine is encoded by one or more dumbbell-shaped DNA minimal vectors.

In some embodiments, a payload nucleic acid encodes and/or expresses (or is the complement of a nucleic acid that encodes or expresses) a vaccine to a pathogen. In some embodiments, a payload nucleic acid encodes and/or expresses (or is the complement of a nucleic acid that encodes or expresses) a vaccine to a bacteria. In some embodiments, a payload nucleic acid encodes and/or expresses (or is the complement of a nucleic acid that encodes or expresses) a vaccine to a virus. Pathogen vaccines involve displaying a pathogen-specific antigen to a subject's immune system such that the immune system is able to more effectively recognize foreign pathogens. One of ordinary skill in the art will be able to select a pathogen-specific antigen for any particular pathogen using methods known in the art.

In some embodiments, a pathogen vaccine is encoded by one or more DNA molecules. In some embodiments, a pathogen vaccine is encoded by one or more plasmids. In some embodiments, a pathogen vaccine is encoded by one or more expression vectors. In some embodiments, a pathogen vaccine is encoded by one or more mRNA molecules. In some embodiments, a pathogen vaccine is encoded by one or more minicircles. In some embodiments, a pathogen vaccine is encoded by one or more dumbbell-shaped DNA minimal vectors.

In some embodiments, a payload nucleic acid is diagnostic. In some embodiments, a payload nucleic acid encodes and/or expresses (or is the complement of a nucleic acid that encodes or expresses) a reporter gene and/or a molecule that is detectable.

### Promoting Oligonucleotide

As described herein, a promoting oligonucleotide is a nucleic acid whose presence is associated with (a) increased level and/or activity of an expression product of a payload; and/or (b) decreased inflammatory and/or otherwise undesirable effect or response (e.g., immune effect or response) associated with administration or delivery of a payload nucleic acid.

In some embodiments, a promoting oligonucleotide is or comprises double-stranded DNA (dsDNA). In some embodiments a dsDNA promoting oligonucleotide is or comprises two DNA strands. In some embodiments, a dsDNA promoting oligonucleotide has a length within a range of 5-200 base pairs. In some embodiments, a dsDNA promoting oligonucleotide has a length of 5, 10, 15, 20, 25, 30, 35, 40, 50, 60, 70, 80, 90, 100, 120, 140, 160, 180, or 200 base pairs. In some embodiments, a dsDNA promoting oligonucleotide has a length of at least 5 base pairs. In some embodiments, a dsDNA promoting oligonucleotide has a length of at most 40 base pairs.

In some embodiments, a promoting oligonucleotide is or comprises single-stranded DNA (ssDNA). An ssDNA promoting oligonucleotide may or may not comprise self-complementary regions. In some embodiments, an ssDNA promoting oligonucleotide comprises one or more stem-loop structures. In some embodiments, an ssDNA promoting oligonucleotide comprises two stem-loop structures (e.g., a ribbon shaped promoting oligonucleotide). In some embodiments, an ssDNA promoting oligonucleotide has a length within a range of 5-100 nucleotides. In some embodiments, an ssDNA promoting oligonucleotide has a length of 5, 10, 15, 20, 25, 30, 35, 40, 50, 60, 70, 80, 90, 100, 120, 140, 160, 180, or 200 nucleotides. In some embodiments, an ssDNA promoting oligonucleotide has a length of at least 5 nucleotides. In some embodiments, an ssDNA promoting oligonucleotide has a length of at most 40 nucleotides.

In some embodiments, a promoting oligonucleotide is or comprises a single RNA strand. An RNA promoting oligonucleotide may or may not comprise self-complementary regions. In some embodiments, an RNA promoting oligonucleotide has a length within a range of 5-100 nucleotides. In some embodiments, an RNA promoting oligonucleotide has a length of 5, 10, 15, 20, 25, 30, 35, 40, 50, 60, 70, 80, 90, 100, 120, 140, 160, 180, or 200 nucleotides. In some embodiments, an RNA promoting oligonucleotide has a length of at least 5 nucleotides. In some embodiments, an RNA promoting oligonucleotide has a length of at most 40 nucleotides.

In some embodiments, a promoting oligonucleotide comprises chemically modified nucleic acid. Chemical modifications may relate to, for example, a nucleotide, a sugar, a base, or a bond of or within a promoting oligonucleotide. In some embodiments, a promoting oligonucleotide comprises at least one phosphorothioate-modified bond. In some embodiments, every nucleotide bond of a promoting oligonucleotide is a phosphorothioate-modified bond. In some embodiments, at most 50% of the nucleotide bonds of the promoting oligonucleotide are phosphorothioate-bonds. In some embodiments, the nucleotide bonds that are phosphorothioate-bonds of the promoting oligonucleotide are at the 5′ and 3′ ends of the nucleic acid sequence.

In some embodiments, phosphorothioate-modified bonds are incorporated into a promoting oligonucleotide to control the oligonucleotide's in vivo half-life (e.g., rate of degradation in a cell, tissue, organism, etc.). In some embodiments, the ratio of phosphorothioate-modified bonds to unmodified bonds in a promoting oligonucleotide is used to control the in vivo half-life. By modifying a promoting oligonucleotide's in vivo half-life, the duration of the oligonucleotide's effects may be controlled. In some embodiments, a promoting oligonucleotide's in vivo half-life is decreased. In some embodiments, a promoting oligonucleotide's in vivo half-life is decreased to minimize constitutive inhibition (e.g., of NF-κB). In some embodiments, a promoting oligonucleotide's in vivo half-life is increased. In some embodiments, a promoting oligonucleotide's in vivo half-life is increased to lessen the quantity of oligonucleotide that is required to achieve a biologic effect.

In some embodiments, a promoting oligonucleotide comprises one or more spacer molecules. In some embodiments, a spacer molecule comprises a linker used to cap the ends of dsDNA and DNA duplexes, such as, for example, hexaethylene glycol.

In many of the embodiments of the present disclosure, a promoting oligonucleotide does not encode for an expression product. The present disclosure surprisingly demonstrates that administration of a promoting oligonucleotide can avoid and/or limit one or more challenges associated with nucleic acid delivery (e.g., a payload nucleic acid).

In some embodiments, a promoting oligonucleotide increases the amount of nucleic acid loaded into a delivery vehicle, especially when the promoting oligonucleotide is co-loaded with a payload nucleic acid in an RBCEV.

In some embodiments, a promoting oligonucleotide can increase the level, expression or activity of a delivered nucleic acid (e.g., or of a product it encodes). In some embodiments, a promoting oligonucleotide increases the number of copies of payload nucleic acid delivered to a system (e.g., a cell, tissue, or organism). In some embodiments, a promoting oligonucleotide increases the number of cells that receive delivery of a payload nucleic acid. In some embodiments, a promoting oligonucleotide increases the amount of expression product expressed per copy of payload nucleic acid. In some embodiments, a promoting oligonucleotide decreases the amount of payload nucleic acid (e.g., or of a product it encodes) degraded upon delivery to a system.

In some embodiments, a promoting oligonucleotide can decrease inflammatory and/or otherwise undesirable effect or response (e.g., immune effect or response) associated with administration or delivery of a payload nucleic acid. In some embodiments, administration of a promoting oligonucleotide decreases expression and/or release of indicative marker(s) of inflammatory and/or otherwise undesirable effect or response (e.g., immune effect or response) associated with administration or delivery of a payload nucleic acid. In some embodiments, administration of a promoting oligonucleotide decreases cytokine expression and/or release associated with administration or delivery of a payload nucleic acid. In some embodiments, administration of a promoting oligonucleotide decreases type I IFN (e.g., IFNa, IFNb, etc.), IL6, CXCL10, and/or CCL2 expression and/or release associated with administration or delivery of a payload nucleic acid.

In some embodiments, a promoting oligonucleotide interacts with a factor endogenous to a cell in which the promoting oligonucleotide has been delivered in order to effect decreased inflammatory and/or otherwise undesirable effect or response (e.g., immune effect or response) associated with administration or delivery of a payload nucleic acid. In some embodiments, a promoting oligonucleotide interacts with a factor endogenous to a cell that typically functions to bind nucleic acid. In some embodiments, a promoting oligonucleotide interacts with a transcription factor. In some embodiments, a promoting oligonucleotide interacts with an RNA-binding protein. In some embodiments, a promoting oligonucleotide interacts with any factor that can be bound by an aptamer.

In some embodiments, a promoting oligonucleotide prevents and/or inhibits an endogenous factor of a cell from interacting with a payload nucleic acid. This prevention and/or inhibition of interaction between an endogenous factor of a cell and a payload nucleic acid by a promoting oligonucleotide may be through direct means (e.g., a promoting oligonucleotide interacting with a factor such that it is unable to interact with a payload nucleic acid) or through indirect means (e.g., a promoting oligonucleotide interacting with a factor that regulates the function or activity of a further factor which might otherwise interact with a payload nucleic acid).

In some embodiments, a promoting oligonucleotide acts as a decoy, lure, trap, bait, mimic, squelch, and/or sink to a factor endogenous to a cell in which the promoting oligonucleotide has been delivered (i.e., acts to absorb and/or neutralize the biologic effects of an endogenous factor such that its endogenous functions are lessened). For example, a promoting oligonucleotide may be or comprise a decoy to a transcription factor; such a decoy could interact with a target transcription factor upon delivery to a cell and decrease the transcription factor's binding to target DNA sequences within the cell's nucleus.

In some embodiments, a promoting oligonucleotide is or comprises a decoy to an effector of a nucleic acid sensing pathway. In some embodiments, a promoting oligonucleotide is or comprises a decoy to an effector of the cGAS-STING signaling axis. In some embodiments, a promoting oligonucleotide is or comprises a decoy to an effector of the TLR9 signaling axis. In some embodiments, a promoting oligonucleotide is or comprises a decoy to an effector of an inflammatory and/or innate immune pathway. In some embodiments, a promoting oligonucleotide is or comprises an NF-κB decoy. In some embodiments, a promoting oligonucleotide is or comprises a decoy to DNA-dependent protein kinase (DNA-PK) and/or poly (ADP-ribose) polymerase (PARP). In some embodiments, a promoting oligonucleotide is or comprises a RIG-I decoy.

### IV. Compositions and Methods of EV Loading

As described herein, loading of an EV (e.g., an RBCEV) with a cargo nucleic acid refers to associating the EV and the cargo nucleic acid in stable or semi-stable form such that the EV is useful as a carrier of the cargo nucleic acid (e.g., allowing its delivery to cells). In some embodiments, cargo nucleic acids are loaded such that they are present in the lumen of the EV. In some embodiments, cargo nucleic acids are attached to, adhered to, inserted through, or complexed with the external surface (e.g., the membrane) of the EV. In some embodiments, cargo nucleic acids are loaded such that there are nucleic acids present in the lumen of the EV and there are nucleic acids attached to, adhered to, inserted through, or complexed with the external surface (e.g., the membrane) of the EV.

In some embodiments, at least one copy of a single cargo nucleic acid is loaded into EVs. In some embodiments, at least one copy each of two different cargo nucleic acids are loaded into EVs. In some embodiments, EVs are loaded with a first cargo nucleic acid, followed by loading of a second cargo nucleic acid. In some embodiments, EVs are loaded first with a payload nucleic acid followed by loading of a promoting oligonucleotide. In some embodiments, EVs are loaded first with a promoting oligonucleotide followed by loading of a payload nucleic acid. In some embodiments, EVs are loaded with two cargo nucleic acids simultaneously. In some embodiments, EVs are loaded simultaneously with a promoting oligonucleotide and a payload nucleic acid.

In some embodiments, methods of EV loading comprise contacting cargo nucleic acid with transfection reagent. In some embodiments, cargo nucleic acid and transfection reagent are brought together under suitable conditions and for suitable time to allow for EV loading to occur. In some embodiments, transfection reagents comprise cationic reagents such as cationic lipid reagents. Transfection reagents may be Lipofectamine™ 3000™ (ThermoFisher), Turbofect™ (ThermoFisher), Lipofectamine™ MessengerMAX™ (ThermoFisher), Exofect™ (System Biosciences), Linear Polyethylenimine Hydrochlorides (e.g., having an average molecular weight of 25,000 Da or 40,000 Da, such as PEIMax™ (Polysciences, Inc.) and jetPEI® (Polyplus transfection)), polybrene or protamine sulfate (see, for example, Delville et al. “A nontoxic transduction enhancer enables highly efficient lentiviral transduction of primary murine T cells and hematopoietic stem cells.” Molecular Therapy-Methods & Clinical Development 10 (2018)).

In some embodiments, loading of cargo nucleic acids into EVs does not comprise viral delivery methods. In some embodiments, loading of cargo nucleic acids into EVs does not comprise a viral vector (e.g., an adenoviral vector, adeno-associated vector, lentiviral vector, retroviral vector, etc.).

### Preparing Cargo Nucleic Acids for Loading

In some embodiments, methods of EV loading comprise a step of preparing the cargo nucleic acid to be loaded. In some embodiments, the preparation step comprises contacting the nucleic acid to be loaded into EVs with transfection reagent under conditions suitable for the formation of a complex between the transfection reagent and the nucleic acid. The nucleic acid and transfection reagent may form a complex (e.g., DNA: PEIMax complex). In some embodiments, the preparation step comprises concentration or dilution of the nucleic acid. In some embodiments, the preparation step comprises addition of buffers or other reagents or media (e.g., Opti-MEM reduced serum media (Gibco)). In some embodiments, the nucleic acid and transfection reagent are contacted for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, or more than 20 minutes. In some embodiments, the preparation step comprises combining a nucleic acid: transfection reagent complex with a further nucleic acid: transfection reagent complex wherein the nucleic acids are non-identical.

In some embodiments, nucleic acid: transfection reagent complexes contain identical nucleic acids. In some embodiments, nucleic acid: transfection reagent complexes contain non-identical nucleic acids in particular ratios. In some embodiments, two non-identical nucleic acid: transfection reagent complexes are combined. The transfection reagent of multiple complexes may or may not be identical. Non-identical nucleic acids may be present in complexes at equimolar amounts (i.e., at an equimolar ratio). Non-identical nucleic acids may not be present in complexes at equimolar amounts (i.e., at an equimolar ratio). The ratio may refer to the amount of a first nucleic acid in relation to a further nucleic acid present in a mixture, wherein the first nucleic acid and further nucleic acid are to be contacted with EVs simultaneously. The ratio may refer to the amount of a first nucleic acid in relation to a further nucleic acid present in a mixture, wherein the first nucleic acid and further nucleic acid are to be contacted with EVs in separate steps.

The first nucleic acid to be loaded and the further nucleic acid to be loaded may be present at a ratio of about 400:1, 300:1, 250:1, 200:1, 150:1, 100:1, 75:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50, 1:75, 1:100, 1:150, 1:200, 1:250, 1:300, 1:400, or 1:500. The first nucleic acid to be loaded and the further nucleic acid to be loaded may be present at a ratio of about 100:1, 75:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50, 1:75, 1:100, 1:150, 1:200, 1:250, 1:300, 1:400, or 1:500. The first nucleic acid to be loaded and the further nucleic acid to be loaded may be present at a ratio of about 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, or 1:25. The first nucleic acid to be loaded and the further nucleic acid to be loaded may be present at a ratio of 1:1.

The first nucleic acid to be loaded and the further nucleic acid to be loaded may be present at a ratio of between 100:1-1:100, 75:1-1:75, 50:1-1:50, 25:1-1:25, 20:1-1:20, 15:1-1:15, 10:1-1:10, 9:1-1:9, 8:1-1:8, 7:1-1:7, 6:1-1:6, 5:1-1:5, 4:1-1:4, 3:1-1:3, 2:1-1:2, or about 1:1.

In some embodiments where three non-identical nucleic acids are to be loaded into EVs, the first, second and third nucleic acids may be present in a ratio of about 1:1:2, 1:1:3, 1:1:4, 1:1:5, 1:1:6, 1:1:7, 1:1:8, 1:1:9, 1:1:10, 1:2:1, 1:3:1, 1:4:1, 1:5:1, 1:6:1, 1:7:1, 1:8:1, 1:9:1, 1:10:1, 2:1:1, 3:1:1, 4:1:1, 5:1:1, 6:1:1, 7:1:1, 8:1:1, 9:1:1, 10:1:1, 1:2:2, 1:3:3. 1:4:4, 1:5:5, 1:6:6, 1:7:7. 1:8:8:1:9:9, 1:10:10, 1:2:3, 1:2:4, 1:3:6, 1:4:8, 1:5:10, 2:4:6, 2:8:4 or other ratio.

In some embodiments, the length of a nucleic acid to be loaded will influence the ratio. In some embodiments, a nucleic acid with longer length will be loaded at a greater ratio than a nucleic acid with less length. In some embodiments, the relative structure of a nucleic acid to be loaded will influence the ratio. In some embodiments, a more compact nucleic acid structure (e.g., a DNA plasmid) will be loaded at a lower ratio than a less compact nucleic acid structure (e.g., a linear DNA). In some embodiments, the strandedness (e.g. single or double) of a nucleic acid will influence the ratio. In some embodiments, a single-stranded nucleic acid will be loaded at a greater ratio than a double-stranded nucleic acid. In some embodiments, a single-stranded nucleic acid will be loaded at a doubled ratio than a double-stranded nucleic acid. The ratio may be adjusted from 1:1 to 2:1 where the first nucleic acid is a single-stranded nucleic acid and the further nucleic acid is a double-stranded nucleic acid.

Loading EVs with Cargo Nucleic Acids

In some embodiments, methods of EV loading comprise a step of loading the EVs with cargo nucleic acid. In some embodiments, prepared nucleic acid: transfection reagent complexes are contacted with the EVs that are to be loaded. In some embodiments, contacting with the EVs is performed subsequently to the contacting of the nucleic acid to be loaded with the transfection reagent. In some embodiments, the nucleic acid: transfection reagent complexes are contacted with a composition comprising a plurality of EVs. In some embodiments, the nucleic acid: transfection reagent complexes and EVs to be loaded are incubated for sufficient time and under appropriate conditions to allow the EV to be loaded with the one or more nucleic acid: transfection reagent complexes. In some embodiments, the nucleic acid: transfection reagent complexes are internalized into the EV. In some embodiments, the nucleic acid: transfection reagent complexes are loaded onto the surface of the EVs (e.g., onto the membranes of the EVs).

In some embodiments, EVs are isolated, washed, and/or concentrated after the step of loading with cargo nucleic acid. In some embodiments, loaded EVs are washed with phosphate buffered saline (PBS). In some embodiments, the washing step is repeated 1, 2, 3, 4, 5, 6, or more times.

In some embodiments, methods of EV loading comprise a temporary or semi-permanent increase in permeability of the membrane of the EVs. Suitable methods to temporarily or semi-permanently increase permeability of the EV membranes are, for example, electroporation, sonication, ultrasound, lipofection or hypotonic dialysis as described in PCT/SG2018/050596 which is herein incorporated by reference in its entirety. In some embodiments, loaded EVs are treated to increase the permeability of the membranes of the EVs. In some embodiments, the loaded EVs are chilled prior to treatment to increase the permeability of the membranes of the EVs. In some embodiments, treatment of the EVs to increase the permeability of the membranes of the EVs further involves one or more buffers (e.g., PBS).

In some embodiments, loading of EVs may be repeated. In some embodiments, EVs are further contacted with nucleic acid: transfection reagent complexes after previous contact with nucleic acid: transfection reagent complexes. In some embodiments, the further nucleic acid: transfection reagent complexes comprise a nucleic acid which is non-identical to the nucleic acid loaded in the previous loading step. In some embodiments, the further loading step is conducted under the same or different time and the same or different conditions as used in the previous loading step. A washing step may be performed after a first loading step and/or subsequent loading steps following the first loading step. Treatment to increase the permeability of the membranes of the EVs may be performed after a first loading step and/or subsequent loading steps following the first loading step.

In some embodiments, EVs are loaded with cargo nucleic acid by electroporation. Electroporation, or electropermeabilization, is a microbiology technique in which an electrical field is applied to cells in order to increase the permeability of the cell membrane, allowing, for example, chemicals, drugs or DNA to be introduced into the cell. In some embodiments, EVs are induced to encapsulate cargo nucleic acids by electroporation. In some embodiments, electroporation involves passing thousands of volts across a distance of one to two millimeters of suspended cells in an electroporation cuvette (1.0-1.5 kV, 250-750V/cm).

In some embodiments, electroporation is a multi-step process with distinct phases. In some embodiments, a first phase comprises application of a short electrical pulse. In some embodiments, voltage settings for a first phase would be within the range of 300-400 mV for less than 1 millisecond across the membrane. Application of the potential may charge the membrane like a capacitor through the migration of ions from the surrounding solution. There may be a rapid localized rearrangement in lipid morphology once the critical field is achieved. The resulting structure may not be electrically conductive but may lead to the rapid creation of a conductive pore. The conductive pores may heal by resealing the bilayer or expand and eventually rupture. In some embodiments, EVs are subjected to electroporation at between about 25 and 300 V or between about 50 and 250 V.

In some embodiments, EVs are loaded with cargo nucleic acid by sonication. Sonication is the act of applying sound energy to agitate particles in a sample. Ultrasonic frequencies (>20 kHz) may be used, leading to the process also being known as ultrasonification or ultra-sonification. Sonication may be applied using an ultrasonic bath or an ultrasonic probe, also known as a sonicator.

In some embodiments, EVs are loaded with cargo nucleic acid by ultrasound. Ultrasound is known to disrupt cell membranes and thereby load cells with molecules. Sound waves with frequencies from 20 kHz up to several gigahertz may be applied to EVs.

In some embodiments, EVs are loaded with cargo nucleic acid by lipofection. Lipofection, or liposome transfection, is a technique used to deliver nucleic acid into a cell by means of liposomes. Liposomes are vesicles that readily merge with phospholipid bilayers as liposomes are made of phospholipid bilayer.

In some embodiments, nucleic acids are loaded at an equimolar ratio when they are of similar size. In some embodiments, nucleic acids are loaded at an equimolar ratio when they are plasmids.

In some embodiments, methods of EV loading comprise removing nucleic acid not contained within the lumen of EVs. In some embodiments, EVs are contacted with DNAse to remove nucleic acid not contained within the lumen of EVs. In some embodiments, EVs are contacted with heparin to dissociate nucleic acid or nucleic acid: transfection reagent complexes.

### V. Methods of Treatment and Uses of EVs

EVs, as described herein, may be useful in methods of treatment. EVs as described herein may be extracellular vesicles derived from red blood cells (RBCEVs).

The present disclosure provides a method of treating and/or preventing an inflammatory disease, disorder, or condition in a human subject comprising administering to the subject a composition comprising a population of extracellular vesicles derived from red blood cells (RBCEVs). Also provided is a composition comprising a population of extracellular vesicles derived from red blood cells (RBCEVs) for use in a method of treating and/or preventing an inflammatory disease, disorder, or condition. Also provided is the use of a composition comprising a population of extracellular vesicles derived from red blood cells (RBCEVs) in the manufacture of a medicament for treating and/or preventing an inflammatory disease, disorder, or condition.

In some embodiments, a composition comprising EVs that have not been loaded with exogenous nucleic acid is useful in methods of treatment. In some embodiments, a composition comprising EVs that have been loaded with exogenous nucleic acid is useful in methods of treatment. In some embodiments, a composition comprising EVs that have been loaded with exogenous nucleic acid is useful in methods of treatment that are known to benefit from administration of nucleic acid. For example, a composition comprising EVs may be useful for delivering a functional gene, or fragment thereof, to replace and/or supplement a gene that is otherwise not fully functional.

RBCEVs disclosed for use in the methods and compositions described herein may be loaded with exogenous nucleic acid. In some embodiments, the exogenous nucleic acid is or comprises an siRNA or an ASO for the gene knockdown of VEGF.

In some embodiments, a composition comprising EVs that have been loaded with exogenous nucleic acid is useful in methods of treatment that are known to benefit from administration of multiple nucleic acids. For example, a composition comprising EVs may be useful for delivering a gene editing system or a vectorized antibody.

A composition comprising EVs may be useful in methods of treatment for a genetic disease, an inflammatory disease, a cancer, an autoimmune disorder, a cardiovascular disease, or a gastrointestinal disease. A composition comprising EVs that have not been loaded with exogenous nucleic acid may be particularly useful in methods of treatment for an inflammatory disease, disorder, or condition.

A composition comprising EVs may be useful in methods of treatment for a cardiovascular disease, disorder, or condition. A composition comprising EVs may be useful in methods of treatment for atherosclerosis. Accordingly, the present disclosure provides a method of treating and/or preventing atherosclerosis in a human subject comprising administering to the subject a composition comprising a population of extracellular vesicles derived from red blood cells (RBCEVs). Also provided is a composition comprising a population of extracellular vesicles derived from red blood cells (RBCEVs) for use in a method of treating and/or preventing atherosclerosis. Also provided is the use of a composition comprising a population of extracellular vesicles derived from red blood cells (RBCEVs) in the manufacture of a medicament for treating and/or preventing atherosclerosis.

In some embodiments, a composition comprising EVs may be useful in treating certain cell types (e.g., target cells). In some embodiments, a target cell for treatment with a composition comprising EVs depends upon the disease, disorder, or condition that is to be treated. In some embodiments, a target cell is related to atherosclerosis. In some embodiments, a target cell is an immune cell. In some embodiments, a target cell is a macrophage. In some embodiments, a target cell is a foam cell.

In some embodiments, a subject treated with a composition comprising EVs has an inflammatory disease. In some embodiments, a subject treated with a composition comprising EVs has cancer. In some embodiments, a subject treated with a composition comprising EVs has an autoimmune disease. In some embodiments, a subject treated with a composition comprising EVs has a cardiovascular disease. In some embodiments, a subject treated with a composition comprising EVs ha a genetic disease. In some embodiments, a subject treated with a composition comprising EVs has a monogenic disease. In some embodiments, a subject treated with a composition comprising EVs has a polygenic disease. In some embodiments, a subject treated with a composition comprising EVs has a physical injury.

In some embodiments, a composition comprising EVs is used for the treatment of cancer. A composition comprising EVs may be useful for inhibiting the growth, proliferation, or survival of cancerous cells. In some embodiments, a composition comprising EVs is used for the treatment of liquid or blood cancer (e.g., leukemia, lymphoma, or myeloma).

In some embodiments, the administration of the composition or medicament comprising a population of RBCEVs is associated with reduced levels of one or more inflammatory cytokines. In some embodiments, the inflammatory cytokines are selected from the group consisting of TNF-α, IL-6, and IL-12.

A composition comprising EVs may be administered, or formulated for administration, by a number of routes, including but not limited to systemic, intratumoral, intraperitoneal, parenteral, intravenous, intra-arterial, intradermal, subcutaneous, intramuscular, oral and/or nasal administration. In some embodiments, a composition comprising EVs is formulated in liquid or solid form. In some embodiments, a liquid formulation is administered by injection to a specific region of the subject or via a specific route of administration.

Administration of a composition comprising EVs may be in a “therapeutically effective amount”, this being sufficient to show benefit to the subject. The amount administered, the rate at which it is administered, and the time-course of administration may depend on the nature and severity of the disease that is to be treated. Prescriptions of treatment (e.g., decisions on dosage) may be within the responsibility of general practitioners and other medical doctors. Prescriptions of treatment may depend on the disease and/or condition that is to be treated, the condition of the individual subject, the site of delivery, the route of administration, and/or other factors. Examples of the techniques and protocols mentioned above may be found in Remington's Pharmaceutical Sciences, 20th Edition, 2000, pub. Lippincott, Williams & Wilkins.

In some embodiments, a composition comprising EVs is administered alone. In some embodiments, a composition comprising EVs is administered in combination with at least one other treatment. A composition comprising EVs may be administered simultaneously or sequentially when administered in combination with at least one other treatment.

In some embodiments, a composition comprising EVs is administered to an animal. In some embodiments, a composition comprising EVs is administered to a mammal. In some embodiments, a composition comprising EVs is administered to a non-human mammal. In some embodiments, a composition comprising EVs is administered to a human. In some embodiments, a composition comprising EVs is administered to a male or female human. In some embodiments, a composition comprising EVs is administered to a human that is a patient. In some embodiments, a composition comprising EVs is administered to non-human animals for veterinary purposes.

### EXEMPLIFICATION

### Example 1: Exemplary Methods

The present Example describes exemplary methods employed in Examples 2-9.

### RBCEV Purification

Whole blood samples were obtained from healthy donors by Innovative Research Inc. (USA). The samples were shipped to Singapore by iDNA (Singapore) and processed by Esco Aster (Singapore), following the protocol from our previous study⁴. Briefly, plasma was removed using centrifugation. Red blood cells were washed twice with PBS by centrifuging at 1000×g for 8 mins to remove plasma proteins. Then, leukocytes were removed using leukodepletion filters (Nigale, China). Washed red blood cells were diluted in PBS containing 0.1 mg/ml calcium chloride and 10 UM calcium ionophore (ab120287, Abcam, USA) and incubated in a cell culture incubator, at 37° C., with 5% CO₂, under humidified conditions overnight. Cells were diluted in PBS on the next day. Red blood cells and cell debris were removed using sequential centrifugation of increasing speeds³. Supernatants containing RBCEVs were collected and filtered through 0.45 μm filter membrane and then spun down at 50,000×g for 1 hour. RBCEVs pellets were further purified by ultracentrifugation with a 60% sucrose cushion at 50,000×g overnight. For long term storage, RBCEVs were resuspended in PBS 4% trehalose, aliquoted, and stored at −80° C.

### RBCEV Ghost Preparation

RBCEVs were resuspended in water at a concentration of 1 mg/ml. The RBCEVs were frozen down at −20° C. and subsequently were thawed at room temperature. A total of three free-thaw cycles were done to achieve adequate depletion of hemoglobin. Expelled hemoglobin was separated from RBCEV membranes by washing using centrifugation at 21,000×g for 1 hour in PBS. The pellet containing RBCEV ghosts was resuspended in PBS and washed once by centrifuging at 21,000×g for 1 hour.

### RBCEV Labeling

For CFSE labeling, RBCEVs, 1 μg/μL, were incubated with 20 μM CFSE (Thermofisher Scientific, USA) in PBS for 3 hours at 37° C. Free CFSE was removed by centrifugation at 21,000×g for 30 mins. RBCEV pellets were resuspended in PBS (0.5 μg of RBCEVs/μL) and centrifuged at 21,000×g for 30 mins. The pellets were then diluted in PBS at 1 mg of RBCEVs/20 mL and left at 4° C. overnight to further elute unbound dyes. CFSE-labeled RBCEVs were concentrated again by centrifugation at 21,000×g for 30 mins.

For Acoerela labeling, RBCEVs, 0.5 μg/μL, were incubated with 2 μM Acoerela dye, a gift from Prof. Bazan Guillermo Carlos's group (National University of Singapore), for 1 hour at room temperature. After labeling, free dye was washed away by centrifugation at 21,000×g for 30 mins. Labeled RBCEVs were washed 3 times, during which, after each centrifugation, the RBCEV pellets were resuspended in PBS (1 μg of RBCEVs/μL) before spinning down again. The supernatant of the last wash served as a flowthrough control.

### Biodistribution Study

Animal experiments were conducted according to our protocols approved by the National University of Singapore's Institutional Animal Care and Use Committee. 500 μg of Aco-490-labeled RBCEVs were injected intravenously in 8-10 weeks old C57BL/6 mice (Invivos, Singapore). After 8 hours, the mice were euthanized, and the livers, lungs, bones and spleens were collected in 10% formalin and left overnight at 4° C. The organs were washed with PBS and transferred into a 15% sucrose solution and subsequently into a 30% sucrose solution. The organs were transferred once they had sunk. The tissues were put in a small container and covered with optimal cutting temperature (OCT) compound and put on dry ice. The frozen tissues were cut into 7 μm-thick sections and mounted on Superfrost slides. The slides were blocked with blocking buffer (2% FBS in PBS) for 40 mins and mouse TruStain (Biolegend, Cat #: 101319) 1:1000 dilution in blocking buffer for 5 mins. Antibodies against mouse F4/80 (Biolegend, Cat #: 123105) or CD169 (Biolegend, Cat #: 142417) (1:500 dilution) were applied and the slides were stained for 1 hour at room temperature and then washed with wash buffer (2% FBS in PBS). Anti-mouse F4/80 antibody is biotinylated. Hence, after staining with this primary antibody, the slides were stained with Streptavidin Alexa 647 (ThermoFisher Scientific, Cat #: S32357) for 1 hour at room temperature and washed with wash buffer (2% FBS in PBS). NucSpot488 (Biotium, Cat #: 40081) (1:2000 dilution) was used to stain nuclei. The sections were washed in PBS and water and then mounted in Vectashield antifade medium (Vector laboratories, Cat #: H-1000-10) and imaged using an Olympus FV3000 confocal microscope.

### Isolation and Differentiation of Peripheral Blood Mononuclear Cells (PBMCs)

Apheresis cones containing blood cells from healthy donors were provided by the Health Sciences Authority in Singapore and processed according to our protocol approved by the Institutional Review Board at the National University of Singapore. PBMCs were then separated by centrifugation with Ficoll-Paque PLUS density gradient (Cytiva, USA) at 700×g for 20 mins with the centrifuge brakes off, followed by three rounds of washing with PBS at 300×g for 8 mins each.

CD14+ monocytes were isolated from PBMCs using a magnetic isolation kit (CD14 MicroBeads, Miltenyibiotec). Then, CD14+ cells were cultured at a concentration of 10⁵ cells per well in 24-well plates in RPMI supplemented with 10% fetal bovine serum (FBS), penicillin (100 IU/ml), streptomycin (100 μg/ml) and 20 ng/ml human M-CSF (BioLegend) for differentiation to macrophages. Cells were maintained with/without RBCEVs for 8 days. For control phenotypes, the polarization of macrophages was induced by incubation with either 20 ng/ml IFN-γ (BioLegend), 100 ng/ml LPS (Sigma-Aldrich) to induce classically activated macrophages (M1) or 20 ng/ml IL-4 (BioLegend) to alternatively activate macrophages (M2) for 1 day. Mheme was induced by incubating macrophages with a combination of 50 nM haptoglobin-hemoglobin complexes for 8 days similarly to RBCEV incubation. Haptoglobin phenotype 1-1 were purchased from Sigma, Singapore (H0138, Sigma). Hemoglobin proteins were prepared from human red blood cells cytosol fraction by one free-thaw cycle. Hemoglobin was further enriched using amicon centrifugation with upper 100 kDa cutoff and lower 10 kDa cutoff.

### PS Blocking Assay

Macrophages were differentiated from CD14+ PBMCs in 20 ng/ml M-CSF for 7 days and incubated with Clipos™ Natural Phosphatidylserine (PS) Lipid Liposomes (CD Bioparticles, USA) (PS liposomes) or Clipos™ Natural Phosphatidylcholine (CD Bioparticles, USA) (PC) Lipid Liposomes (PC liposomes) at different concentrations (110, 220, and 440 μM) for 30 mins. 10 μg of CFSE-labeled RBCEVs were added into each well. 1 hour after incubation, cells were washed and collected, and CFSE signals were analyzed using flow cytometry to measure RBCEV uptake level as described below.

### PS Reduction and Restoration

Phosphatidylserine removal and restoration were based off of a phospholipid exchange method mediated by alpha-cyclodextrin. RBCEVs (250 ng/μL) were incubated with 0.3 mM 1,2-distearoyl-sn-glycero-3-phosphocholine (DSPC) and α-cyclodextrin (40 mM) for 45 mins at 37° C. The mixture was loaded on top of 2 mL of 20% sucrose and centrifuged at 21,000×g for 30 mins to remove excessive lipids after the reaction. Cyclodextrin was washed away from the RBCEVs with PBS by centrifugation for 30 mins at 21,000×g. The PS restoration was done on PS-depleted EVs using the same procedure but instead DSPC, 0.3 mM L-α-phosphatidylserine was used.

### Flow Cytometry

Flow cytometry was applied to investigate surface markers of activated macrophages. Cells were washed with FACS buffer (PBS with 2% FBS and 2 mM EDTA) and blocked with Human TruStain FcX™ (Biolegend, San Diego, USA). Then, cells were incubated on ice with fluorescent antibodies detecting CD11b (FITC), CD80 (PE-Dazzel-594), CD86 (APC), CD206 (PE), CD163 (APC) before being washed in FACS buffer. Fluorescence was analyzed using the flow cytometer Cytoflex LX (Beckman Coulter, USA).

Flow cytometry was also applied to evaluate the uptake of RBCEV by macrophages. In brief, RBCEVs were stained with carboxyfluorescein succinimidyl ester (CFSE) prior to the incubation with cells. After 2 hours, cells were harvested and washed with FACS buffer before fluorescence analysis using the BD LSR Fortessa cytometer (BD Biosciences, USA).

### Nano Flow Cytometry

After PS depletion and restoration, CFSE-labeled RBCEVs (40 ng/μL) were stained with Annexin V-APC (BioLegend, Cat #: 640920) (1:250 dilution) in 100 μL of Annexin V binding buffer for 20 mins at room temperature. The samples were washed using centrifugation and resuspended in 200 μL of Annexin V binding buffer. Annexin V signals were analyzed using Nanoparticle flow cytometry on Cytoflex LX. Particles were detected using violet side scatter and RBCEVs were gated on the CFSE-positive population. From the CFSE-positive population, Annexin V signals were analyzed.

### Cell Culture

THP1, HEK-293T (293T), Hela, and NCI-H358 (H358) cells were purchased from the American Type Culture Collection (ATCC, USA). MCF10CA1a (CA1a) cells were purchased from the Karmanos Cancer Institute (Wayne State University, USA).

Cells were kept at 37° C. and 5% CO₂ in a humidified incubator. CA1a, 293T, HeLa, and H358 cells were maintained in DMEM High Glucose w/L-Glutamine w/Sodium Pyruvate (ThermoFisher Scientific). THP1 cells were maintained in RPMI 1640, where both media were supplemented with 10% FBS, 1% Penicillin-Streptomycin, and 5 μg/ml Plasmocin™ prophylactic.

### RBCEV Uptake Assay

Macrophages were incubated with 20 μg of RBCEVs in 400 μl of culture medium for 2 hours at 37° C. or 4° C. The medium was aspirated, the cells were rinsed once with cold PBS, and detached by incubation with 0.25% Trypsin-EDTA (ThermoFisher Scientific) for 10 mins at 37° C. The cells were washed twice with FACS buffer by centrifugation for 5 mins at 300×g at 4° C. before being analyzed by flow cytometry.

### Absolute Quantification of RBCEV Uptake

Cells were incubated with 40 μg of CFSE-EVs in 500 μl of culture medium for 2 hours at 37° C. (or 4° C., for the binding control). For adherent cells, the culture medium was aspirated, the wells were rinsed once with PBS and the cells were detached by incubation with 0.25% Trypsin-EDTA (ThermoFisher Scientific) for 10 mins at 37° C. THP1 cells, which are suspension cells, were first collected and washed once with PBS+2% FBS by centrifugation for 5 mins at 300×g at 4° C. before being similarly treated with 0.25% Trypsin-EDTA. All cells were then washed twice with PBS+2% FBS and counted using C-slides (NanoEntek, South Korea) and the Countess® II FL Automated Cell Counter (ThermoFisher Scientific). Cells from each sample were aliquoted into a well on a 96-well plate and topped up to a total of 200 μL with PBS+2% FBS and 2% Triton X-100 (Sigma-Aldrich). CFSE fluorescence intensity was measured at 482 nm excitation and 527 nm emission using a Tecan Spark 10M Microplate Reader (Tecan, USA). The mass of EVs was calculated from the CFSE fluorescence intensity using a standard curve constructed from a series of dilutions of known CFSE-EV concentrations. EV mass was then converted to EV number by multiplying with 1.32×10⁹ (average number of RBCEVs per 1 μg).

### Immunofluorescence Staining

Macrophages differentiated from PBMCs on cover slips were treated with RBCEVs and fixed at different timepoints with 10% formalin. The cells were then washed with PBS containing 2% FBS prior to permeabilization with 0.1% Triton X-100. The cells were then incubated with the appropriate primary antibody against markers for early endosomes, late endosomes, or lysosomes-late endosomes (i.e., EEA, LBPA and LAMP1, respectively), followed by incubation with the appropriate secondary antibody (AlexaFluor 488/594/647-conjugated mouse I) prior to imaging with the Olympus FV3000 confocal microscope (Olympus Corporation). Primary antibodies used for immunofluorescent staining were anti-LAMP1 antibody (Abcam, Cat #: ab25630 or Cell Signaling Technology, Cat #: 9091S), anti-EEA antibody (Cell Signalling Technology, Cat #: 2411S), anti-LBPA (Sigma-Aldrich, Cat #: MABT837), anti-SLC48A1 (HRG1) (Thermofisher Scientific, Cat #: PA5-42191), and anti-human BAND 3 (Santa Cruz Biotechnology, Cat #: sc-133190).

### RBCEV Binding Assay

Macrophages were detached from plates by incubation for 10 mins at 37° C. with 0.25% Trypsin-EDTA (ThermoFisher Scientific), Accutase® Cell Detachment Solution (BioLegend, USA), FACS buffer, or PBS with 2% FBS only and collected by pipetting. The cells were then washed twice with PBS with 2% FBS by centrifugation at 300×g at 4° C. for 5 minutes and incubated with 10 μg of RBCEVs in 100 μL of PBS with 2% FBS for 15 minutes on ice. Two rounds of washing with FACS buffer were performed and the cells were resuspended in the same buffer for flow cytometry analysis.

### Western Blot

RBCEV pellets were lysed in RIPA buffer and incubated on ice for 10 mins. 4× Laemmli buffer was added to the lysate and the mixture was incubated at 95° C. for 5 mins. RBCEVs protein samples were loaded on sodium dodecyl sulphate-polyacrylamide gel electrophoresis (SDS-PAGE) gels and transferred to a Polyvinylidene fluoride (PDVF) membrane. The membrane was blocked with 5% skimmed milk in Tris-buffered saline with 0.1% Tween 20 detergent (TBS-T) before adding primary antibodies. After overnight incubation at 4° C., the membrane was washed with TBS-T and probed with Horseradish peroxidase (HRP)-conjugated secondary antibodies. Then, Enhanced Chemiluminescence (ECL) was added to the membrane before imaging using a ChemiDoc™ XRS+ system (Bio-Rad).

qPCR

Total RNA was extracted from cells using TRIzol™ Reagent (ThermoFisher Scientific) according to the manufacturer's instructions. RNAs were converted to cDNA using a high-capacity cDNA reverse transcription kit (ThermoFisher Scientific) and quantified with Ssofast® Green qPCR kit (Bio-Rad), normalized to the expression of GAPDH, according to the manufacturers' protocols. All qPCR reactions were performed using a CFX96 Touch™ Real-Time PCR Detection System (Bio-Rad) or a QuantStudio 6 Flex Real-Time PCR System (Life Technologies).

### OxLDL Treatment, Oil Red-O Staining, Imaging, and Quantification

PBMCs after differentiation and stimulation with indicated conditions on cover slips in 24-well plates were treated with Low Density Lipoprotein from Human Plasma, oxidized (oxLDL; Athens Research & Technology, USA) at 20 μg/ml for 24 hours. Cells then were either collected at the indicated timepoint or the media were gently replaced with new media supplemented with RBCEVs or human plasma for another 24-hour incubation before fixing with 10% formalin.

Oil red O with concentration of 0.3% was prepared for staining cell at room temperature in 10 minutes. Then, cells were washed with deionized water before imaging under the microscope. Quantification was calculated performed using ImageJ according to the number of stained cells in at least 5 random areas for each sample.

### Atherosclerotic Mouse Model Study

ApoE−/− mice were obtained from Jackson Laboratory (Maine, USA). Male mice were on a chow diet for 4 weeks. From week 5, mice were fed with a high-fat diet (TD.88137, Teklad) for 8 weeks. Concurrently, RBCEVs were administered intravenously at the dose of 50 mg/kg twice per week. Control mice were injected with the same volume of PBS as RBCEV injection volume (100 μL). After 8 weeks of the high-fat diet, the aortas were harvested under the microscope to remove adipose and connective tissues. The aortas were fixed in formalin overnight at 4° C. and subsequently stained with ORO and imaged for quantification. Aortic roots were fixed in formalin, embedded on OCT, and stored at −80° C.

### Oil Red O Staining and Imaging of Aorta

After fixation, the aortas were washed with PBS once and subsequently washed twice with 60% isopropanol for 5 mins each on a rotating shaker. The aortas were transferred to a 60% isopropanol solution containing 0.3% ORO and incubated for 1 hour at room temperature on a rotating shaker. After staining, the samples were washed with PBS. All excessive adipose and connective tissues were removed under the microscope and the aortas were cut open on a black mat. Aortas' images were taken using a stereo microscope (Nikon Instrument Inc., Tokyo, Japan) connected to a digital camera (Olympus DP22, Olympus Corporation, Tokyo, Japan). Total plaque area was analysed from the images using ImageJ, with the color threshold analysis method.

### Staining of Aortic Root

Aortic roots from the ApoE−/− mice fed and treated as described above were collected and fixed in 10% formalin overnight at 4° C. The aortic roots were washed with PBS and transferred into a PBS solution containing 15% sucrose and subsequently to a PBS solution containing 30% sucrose. The organs were transferred to the next solution once they had sunk to the bottom of the container. The tissues were then transferred to a small cryomold, covered with the optimal cutting temperature (OCT) compound and put on dry ice. The heart tissues containing aortic roots were sectioned horizontally toward the aortic cusp. Once the aortic cusp appeared, sections were collected and counted from 1 to 55. Sections 1, 13, and 25 served as controls for staining. Sections 4, 7, 10, 16, 19, 22, 28, 31, 34, 39, 45, and 51 were used for staining of HO-1 and CD68. The sections were mounted on Superfrost slides. The slides were blocked with the blocking buffer (PBS containing 2% FBS) for 40 minutes and then incubated with the blocking buffer containing mouse TruStain (BioLegend, Cat #: 101319) at a dilution of 1:1000 for 5 minutes. Antibodies against mouse HO-1 (Proteintech, Cat #: 10701-1-AP) (1:400 dilution in PBS 2% FBS, 0.1% Triton-X100) and against mouse CD68 (Bio-Rad, Cat #: MCA1957) (1:600 dilution in PBS 2% FBS, 0.1% Triton-X100) were applied and the slides were incubated for 1 hour at room temperature and then washed with the wash buffer (PBS containing 2% FBS). AlexaFluor 488-Anti mouse IgG secondary antibody (Jackson ImmunoResearch, Cat #: 711-545-152) and AlexaFluor 594-Anti rat IgG secondary antibody (1:500 dilution in in PBS 2% FBS, 0.1% Triton-X100) were applied for 1 hour at room temperature and then washed with the wash buffer. The nuclei were stained with Hoechst 33342 (1 g/mL, Life Technologies) in PBS for 10 min at room temperature. The sections were then treated with Vector® TrueVIEW® Autofluorescence Quenching Kit (SP-8400-15), followed by washing with PBS and water. The slides were then mounted under coverslips using the Vectashield antifade medium (Vector laboratories, Cat #: H-1000-10) and imaged using the Leica Thunder Imager (10× objective lenses).

### Primers

All primers were synthesized by Integrated DNA Technologies (USA).

GAPDH forward:
GTCTCCTCTGACTTCAACAGCG
GAPDH reverse:
ACCACCCTGTTGCTGTAGCCAA
ABCA1 forward:
TGTCCAGTCCAGTAATGGTTCTGT
ABCA1 reverse:
AAGCGAGATATGGTCCGGATT
ABCG1 forward:
TGCAATCTTGTGCCATATTTGA
ABCG1 reverse:
CCAGCCGACTGTTCTGATCA
HO-1 forward:
CTTCACCTTCCCCAACATTG
HO-1 reverse:
CTTGCAACTCCTCAAAGAGC
IL-1β forward:
CCACAGACCTTCCAGGAGAATG
IL-1β reverse:
GTGCAGTTCAGTGATCGTACAGG
IL-10 forward:
CCTGCCTAACATGCTTCGAG
IL-10 reverse:
CTCAGACAAGGCTTGGCAAC
LXRb forward:
CTTCGCTAAGCAAGTGCCTGGT
LXRb reverse:
CACTCTGTCTCGTGGTTGTAGC
TNF-α forward:
CCTCTCTCTAATCAGCCCTCTG
TNF-α reverse:
GAGGACCTGGGAGTAGATGAG
ASOs
HRG1-ASO (5′->3′):
CCTCCAATAATCTTGCATGT+C*/i2MOErC/*
/i2MOErT/*/i2MOErC/*/i2MOErC/*A*A
*T*A*A*T*C*T*T*G*/12MOErC/*
/i2MOErA/*/i2MOErT/*/i2MOErG/*+T
HO-1 ASO (5′-3′):
ATCACCAGCTTGAAGCCGTC+A*/i2MOErT/*/
i2MOErC/*/i2MOErA/*/i2MOErC/*C*A*
G*C*T*T*G*A*A*G*/12MOErC/*/i2MOErC/
*/i2MOErG/*/i2MOErT/*+C
NC-ASO:
CGACTATACGCGCAATATGG+C*/i2MOErG/*
/i2MOErA/*/i2MOErC/*/i2MOErT/*A*T
*A*C*G*C*G*C*A*A*/12MOErT/*/
i2MOErA/*/i2MOErT/*/i2MOErG/*+G

In which:
A, T, C, G: nucleotides
/i2MOEr/:2′-OMe modification at internal nucleotide.
*: phosphorothioate (PS) modification

### Example 2: RBCEV Uptake is Cell Type-Dependent Both In Vitro and In Vivo

The present Example provides quantitative assessments of RBCEV uptake in particular cells and tissues in vivo. Among other things, the present Example provides technologies that compare and optionally quantify extent of RBCEV uptake among different cell types, and/or for assessing quantity of cargo uptake (e.g., delivered via RBCEVs). The present Example particularly provides technologies for assessing uptake into macrophages.

Published reports have demonstrated that RBCEVs were highly accumulated in the liver, spleen, lung, and bone marrow after intravenous injection in C57BL/6 mice³. Distribution at a cellular level was not assessed, however.

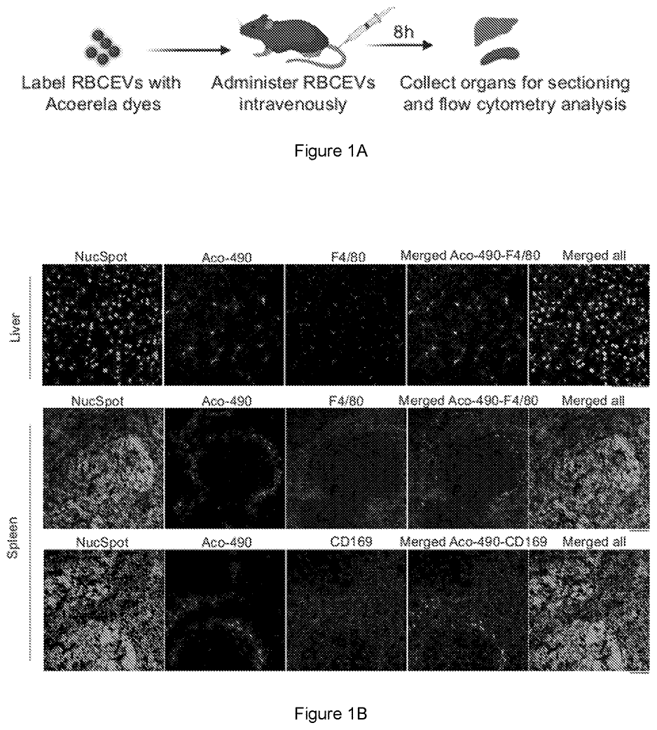

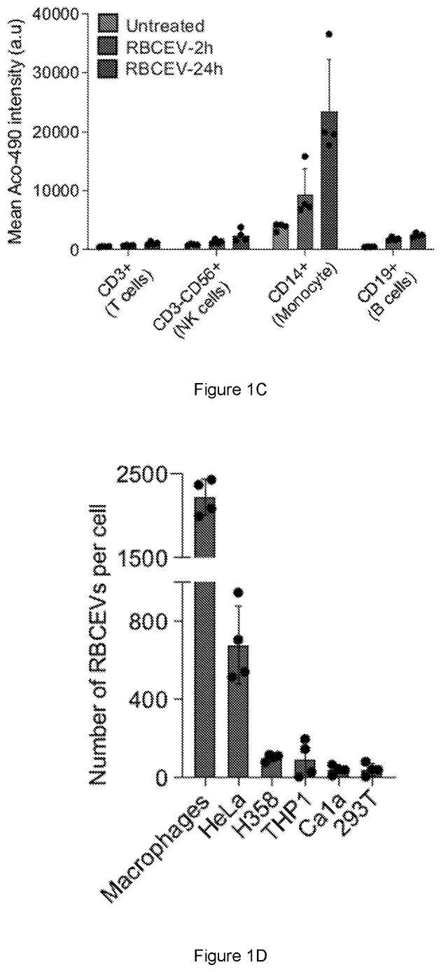

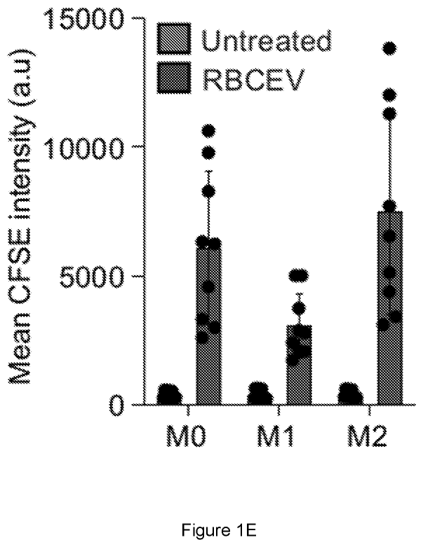

The present Example describes a detailed analysis of RBCEV distribution at the cellular level. To track the distribution of RBCEVs in vivo, we used Acoerela dye Aco-490, a water soluble and fluorogenic, lipophilic dye. This new class of dyes are based off of conjugated oligoelectrolytes (COEs) which have been previously shown to preferentially stain lipid bilayers^{20,21}. Aco-490 has been specifically tuned for excitation at 405 nm and emission at 525 nm. Livers, spleens, lungs, and femur bones were collected 8 hours after intravenous injection of Aco-490-labeled RBCEVs (FIG. 1a). These organs were fixed, cryo-sectioned and stained with mouse resident-macrophages' surface markers (F4/80, and CD169). Nuclei were stained with Nucspot 488. In line with previous studies^{4,7}, we found that RBCEVs mainly accumulated in the liver and spleen (FIG. 1b). No visible Aco-490 signal was detected in sections of lung and bone tissues. In the liver sections, most of the Aco-490 fluorescent signals were colocalized with F4/80+ cells, suggesting that RBCEVs were extensively taken up by liver Kupffer cells, with little uptake by other cell types. In the spleen, RBCEVs were mostly distributed to the marginal zone, resulting in a ring pattern within the spleen section. Staining with F4/80 showed few events of colocalization with Aco-490 signals, as opposed to a prominent colocalization of Aco-490 signals with the CD169+ cells in the marginal zones.

We also examined if RBCEVs were taken up by different cell types in the circulation. Peripheral blood mononuclear cells (PBMCs) were incubated with RBCEVs for 2 hours or 24 hours, then stained with different cell surface markers and analyzed using flow cytometry. CD14+ monocytes took up RBCEVs the most, while B cells (CD19+) and NK cells (CD3−CD56+) took up significantly fewer RBCEVs. T cells (CD3+) showed the lowest uptake of RBCEVs, with almost no uptake detected after 2 hours of incubation (FIG. 1c).

We further demonstrated that extent of RBCEV uptake can vary greatly among different cell types, this time by comparing the uptake capacity of several cancer cell lines (FIG. 1d). To determine uptake level of RBCEVs, we developed an absolute quantification method. After an incubation with CFSE-labeled RBCEVs (CFSE-EVs), cells were trypsinized, washed, and counted. Then, cells were lysed in 2% Triton-X100 and the CFSE fluorescent signals were measured using a plate reader. Using a standard curve of known EV concentrations, the amount of RBCEVs taken up by each cell line was calculated based on CFSE signals and normalized by the cell number. Our data showed that after 2 hours of incubation with 40 μg of RBCEVs at 37° C., the uptake level among cancer cells varies greatly. On average, each CA1a cell took up ˜200 EVs while Hela cell took up ˜500 vesicles. Uptake by macrophages was surprisingly higher, up to ˜2000 EVs per cell. The present Example therefore demonstrates that this method can effectively obtain quantitative data. The present disclosure provides an insight that such quantitative data may be particularly useful for pharmacological assessment of RBCEVs, e.g., in the context of therapeutic development and/or of treatment (FIG. 1d).

We further investigated if different subtypes of macrophages might exhibit differences in their capacity for EV uptake. Indeed, upon polarization of monocyte-derived macrophages, we found that MO and M2 macrophages took up significant amounts of RBCEVs, while M1 macrophages showed a slightly lower capacity for RBCEV uptake (FIG. 1e).

Certain reports have indicated that, following intravenous injections in mice, various types of EVs, including RBCEVs, can accumulate in the liver and spleen^{1,5,20}. We observe that these organs have large populations of macrophages, and propose that RBCEVs may provide a particularly effective strategy for delivery of payloads to, or for otherwise impacting, macrophages. Indeed, the present disclosure demonstrates macrophages differentiated in vitro from human monocytes were highly efficient at taking up RBCEVs. We provided the first quantitative estimate of ^{˜}2,221 (±106.1, SEM) RBCEVs taken up per macrophage after a 2-hour incubation with 80 ng/μL RBCEVs.

Provided technologies are useful in a variety of contexts including, for example, for in vitro uptake of RBCEVs into macrophage cells (e.g., for delivery of endogenous or exogenous RBCEV cargo); macrophages that uptake such RBCEVs may be subjected to one or more assessments and/or may be useful, for example, for therapeutic purposes. In some embodiments, provided technologies facilitate or permit (i) comparison of extent of RBCEV uptake among different cell types, (ii) estimates of quantity of a given cargo taken up (e.g., along with RBCEVs) into macrophages and/or (iii) pharmacological assessment of RBCEV-mediated therapeutic delivery.

### Example 3: RBCEVs are Taken Up Robustly by Macrophages in a Process Mediated by Phosphatidylserine

The present Example provides insight(s) that RBCEV uptake in particular cells is mediated at least in part by phosphatidylserine. Among other things, the present Example provides technologies that compare and optionally quantify extent of RBCEV uptake by different mechanisms, and/or for modulating the amount of RBCEV uptake. The present Example particularly provides technologies relating to uptake into macrophages.

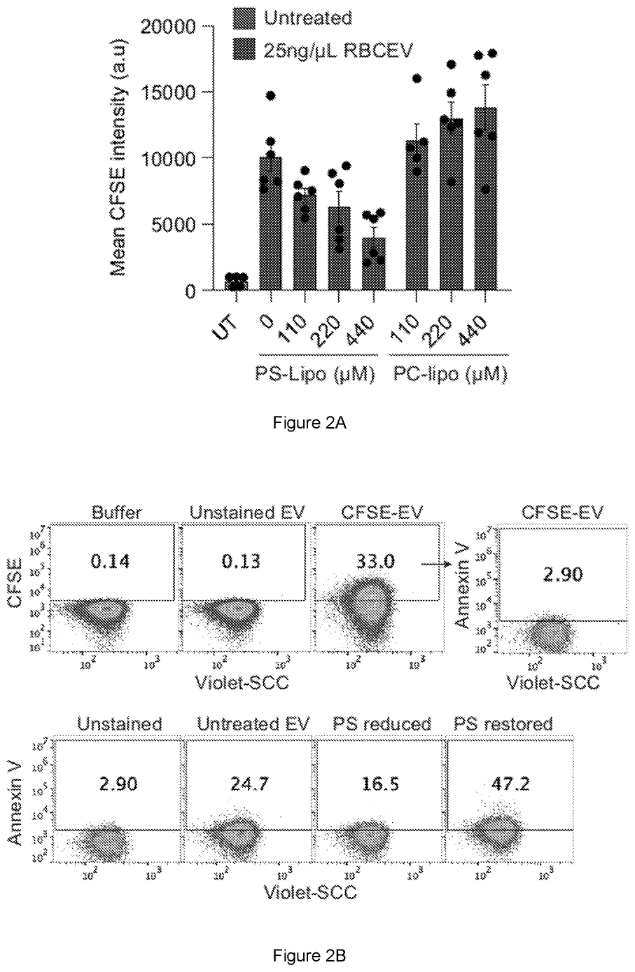

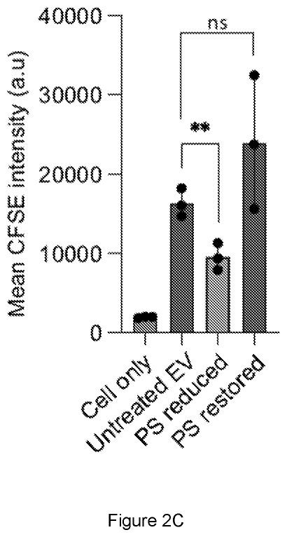

To assess the role of phosphatidylserine (PS) in mediating uptake of RBCEVs by macrophages, we saturated PS receptors on macrophages using PS liposomes. As a control, we replaced PS liposomes with the same concentration of phosphatidylcholine (PC) liposomes. After 30 minutes of incubation with PS liposomes and PC liposomes, CFSE-labeled RBCEVs were added to CD14+ PBMC-derived macrophages. Cells were incubated with RBCEVs for 2 hours at 37° C. and analyzed by flow cytometry to measure the CFSE signals. Our data showed that PS but not PC liposomes blocked uptake of RBCEVs by macrophages in a dose-dependent manner. PS liposomes at a concentration of 440 μM reduced more than half the uptake of RBCEVs compared with no blocking or control liposomes. However, the uptake level had not reached a plateau for all concentrations we tried, suggesting further reductions in the uptake of RBCEVs could be achieved with more extensive blocking (FIG. 2a).

We also investigated how removing PS from RBCEVs influenced RBCEV uptake by macrophages. Following labeling with CFSE, RBCEVs were treated with α-cyclodextrin and 1,2-distearoyl-sn-glycero-3-phosphocholine (DSPC) to reduce PS on their outer leaflet membrane (“PS reduced”), and L-α-phosphatidylserine was added to the PS-reduced RBCEVs to restore PS expression (“PS restored”). Compared to untreated RBCEVs, annexin V staining was lower in the “PS reduced” group and higher in the “PS restored” group, demonstrating the effect of these two PS manipulation procedures (FIG. 2b). Treated and control CFSE-labeled RBCEVS were then added to macrophages and incubated for 2 hours. As expected, differences in CFSE signals between groups indicate that PS-reduced RBCEVs were taken up to a lesser degree than normal RBCEVs. Meanwhile, adding PS back to the PS-reduced RBCEVs not only rescued but enhanced uptake compared to normal RBCEVs (FIG. 2c). The present disclosure demonstrates that RBCEV uptake by macrophages can be strongly mediated by PS (e.g., by interactions between RBCEVs and PS receptors on cells).

The present disclosure probes the molecular interactions at cell-EV interface that are responsible for initiating EV engulfment. Certain studies have suggested that scavenger receptors, on mouse and human macrophages, may play a role in mediating EV uptake in a process similar to recognition and phagocytic clearance of apoptotic cells^{29,30}. For RBCEVs, Zhang et al. indicated that EV accumulation in mouse liver was macrophage-dependent and mediated by the complement protein Clq⁷. The present disclosure describes that blocking PS receptors on macrophages with PS liposomes can greatly reduce uptake of RBCEVs. Similarly, uptake was significantly inhibited when we reduced the presence of PS on RBCEVs, whereas restoring PS on RBCEVs leads to increased uptake.

Without wishing to be bound by any particular theory, we note that anti-inflammatory effects of RBCEVs might derive from phosphatidylserine on the RBCEV plasma membrane and/or one or more products of heme degradation. PS has been described to possess anti-inflammatory properties in certain instances, such as PS-dependent anti-inflammatory responses induced by apoptotic cells³⁴. Contacting macrophages with PS liposomes has been shown to reduce expression of TNFα and the surface marker CD86 while stimulating secretion of TGFβ and IL-10³⁵.

The present disclosure describes that contacting macrophages with RBCEVs results in anti-inflammatory effects. For example, macrophages contacted with RBCEVs showed a strong upregulation of HO-1. HO-1 has been shown, in some cases, to activate anti-inflammatory pathways. The mechanistic basis of its anti-inflammatory activity partly relies on its catalytic product, carbon monoxide (CO), which is generated upon heme degradation. Stimulating macrophages with CO or overexpression of HO-1 results in significant reduction of TNFα and IL-1β secretion in an LPS-induced inflammatory model¹². Taken together, these data suggest that the anti-inflammatory effects of RBCEVs might result from phosphatidylserine and/or endogenous hemoglobin, which can induce HO-1 expression in macrophages. Preventing and/or ameliorating inflammation with technologies mediated by macrophages and described herein might be useful for treatment of multiple diseases, including but not limited to, atherosclerosis. For instance, it has been shown that treatment of macrophages with PS liposomes might improve cardiac repair³⁵. Further, driving macrophages from an M1- to an M2-like phenotype might be a strategy to treat diseases related to tissue repair and regeneration³⁶. The present disclosure provides therapeutic strategies which use EVs as natural anti-inflammatory drugs to treat immune-related diseases, especially chronic inflammation. The present disclosure provides therapeutic strategies which use EVs as natural anti-inflammatory drugs to improve upon existing treatment treatments for immune-related diseases, which typically require long-term treatment with high risk for side effects and complications.

### Example 4: RBCEVs are Internalized Mainly Through Endocytosis (Including Phagocytosis) and Partially Through Direct Fusion

The present Example provides insight(s) that RBCEV uptake in particular cells is mediated mainly through endocytosis and partially through direct fusion. Among other things, the present Example provides technologies that compare and optionally quantify extent of RBCEV uptake by different mechanisms, and/or for modulating the amount of RBCEV uptake. The present Example particularly provides technologies relating to uptake into macrophages.

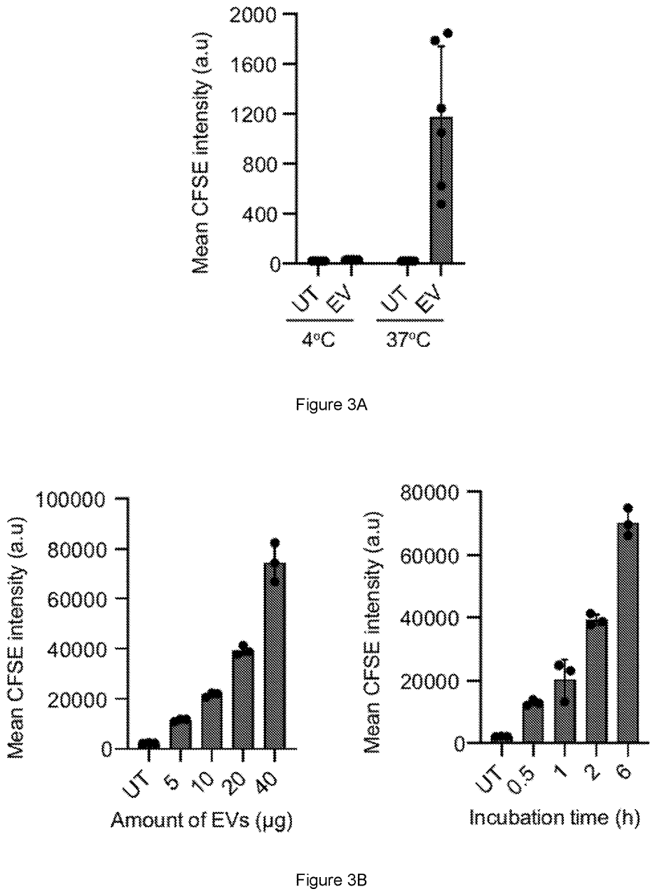

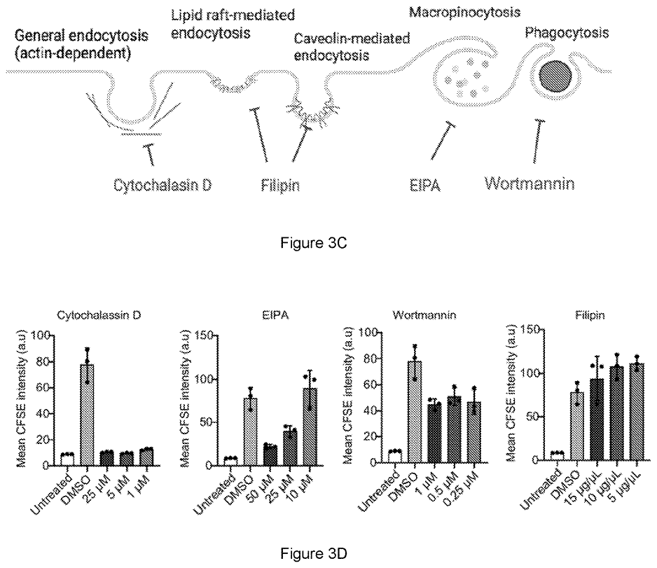

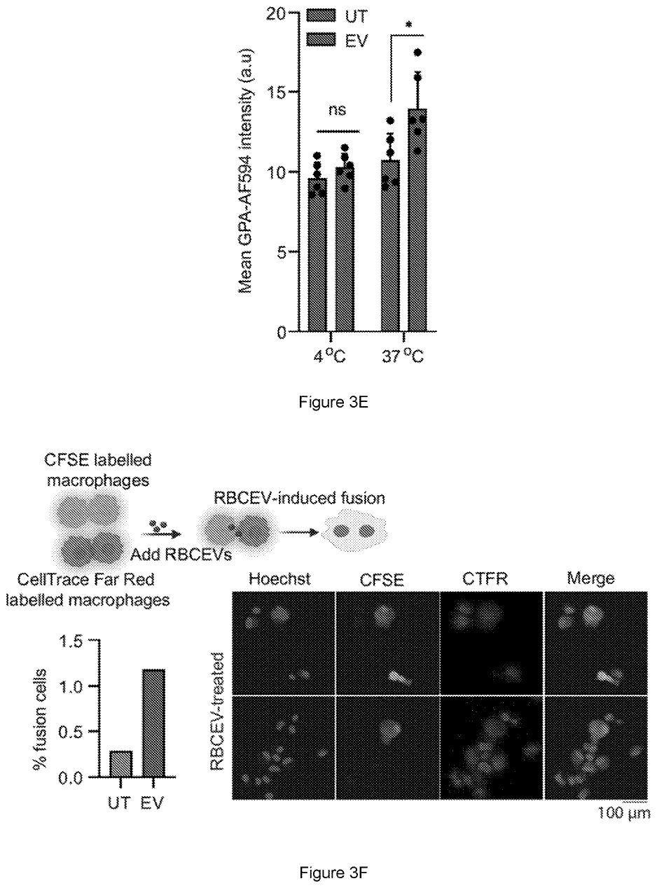

EVs might be taken up into cells by different routes. The fate of EVs and/or EV cargo within a recipient cell might be influenced by which route(s) of uptake are predominant and/or available in certain cases. For example, EV and/or EV cargo half-life, spatial kinetics, downstream biological effects, concentration, etc. might be influenced by route of uptake. Thus, we further investigated how RBCEVs are taken up by macrophages, firstly by assessing if uptake is an active, energy-dependent process or if it happens passively. Our data showed that incubating macrophages with RBCEVs at 4° C. significantly inhibited the uptake of RBCEVs compared with an incubation at 37° C., indicating that uptake of RBCEVs is an active process (FIG. 3a). At 37° C., the uptake of RBCEVs by macrophages is also time-dependent and concentration-dependent (FIG. 3b).

We next used several pharmacological inhibitors to test which active endocytosis processes are important for RBCEV uptake. Disruption of actin microfilaments using Cytochalasin D substantially reduced uptake of RBCEVs almost to background level (FIGS. 3c and 3d). EIPA, a macropinocytosis inhibitor, prevented uptake of RBCEVs in a dose-dependent manner. The phagocytosis inhibitor Wortmannin also reduced the uptake of RBCEVs significantly. There was no reduction in RBCEV uptake when cells were treated with filipin, which blocks caveolin-mediated endocytosis (FIGS. 3c and 3d). Overall, our data suggested that RBCEVs are taken up mainly though endocytosis and strongly correlated with macropinocytosis and phagocytosis.

Surprisingly, we also found supporting evidence for a direct fusion between RBCEVs and the plasma membrane of macrophages. After incubation with RBCEVs at 37° C., macrophage surfaces stained positive for GPA, a protein marker of RBCEVs, while there was no detectable GPA staining after incubation at 4° C. If this signal came from binding of RBCEVs to macrophages, there would be almost equal GPA staining of cells incubated at 37° C. and 4° C., since binding can take place at both temperatures. The fact that GPA staining was present only after the incubation at 37° C. suggests that some RBCEVs can undergo fusion directly with the plasma membrane. To test this hypothesis, we stained macrophages with either CFSE or CellTrace Far Red (CTFR), mixed these two populations at 1:1 ratio, and cultured them at high cell densities to maximize the cell-to-cell contact, with or without RBCEVs. At dense concentrations of cells, fusion of one EV could happen with two adjacent cells and result in the fusion of the two cells. If two cells were fused, they would become one cell with two nuclei. A cell which resulted from a fusion event would have both CFSE and CTFR signals, if the two parental cells were stained with CFSE and CTFR. A cell resulting from fusion might alternatively only have CFSE or CTFR signals if the two parental cells were stained with the same dye. Our data revealed a significant increase in fusion events of RBCEV-treated macrophages, resulting in cells with two nuclei, compared with untreated macrophages (FIG. 3e). However, these events were scarce, as only ˜1% of cells fused. The present disclosure demonstrates that RBCEVs might directly fuse with the macrophage's plasma membrane at very low frequencies, but RBCEVs are mainly taken up by macrophages through endocytosis.

Previous studies have indicated that endocytosis and actin remodeling might contribute to cellular internalization of EVs^{25,26}. The present disclosure demonstrates that, among the possible endocytosis pathways, macropinocytosis and phagocytosis are likely the major routes for RBCEV entry; meanwhile, blocking lipid rafts with Filipin failed to inhibit RBCEV uptake.

It was not previously appreciated whether or not EVs could be taken up into cells by directly fusing with the plasma membrane. A number of studies have reported the ability of EV membranes to fuse with cellular lipid bilayers, resulting in a hybrid membrane and mixing of contents, but this typically requires a low pH, such as that in lysosomes^{27,28} Here, we surprisingly found that mixing macrophages at high cell densities with RBCEVs resulted in a higher rate of cell fusion than without RBCEVs, which indicates that RBCEVs can merge two cells in close proximity by fusing with the plasma membranes of both cells. Such fusion events were rarely recorded in our assay, however, thus active endocytic processes remain the major route for EV uptake.

### Example 5: RBCEVs Accumulate in Late Endosomes and Lysosomes

The present Example provides insight(s) that RBCEV uptake in particular cells results in RBCEV accumulation in late endosomes and lysosomes. Among other things, the present Example provides technologies that compare and optionally quantify extent of RBCEV and/or RBCEV cargo localization in different subcellular compartments (e.g., organelles and/or intracellular vesicles). The present Example particularly provides technologies relating to uptake into macrophages.

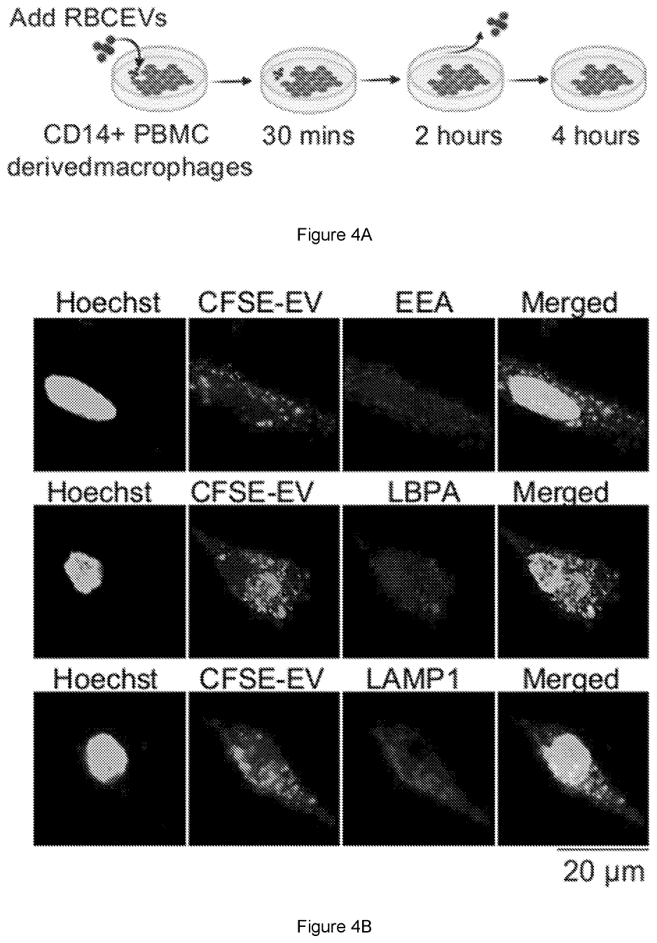

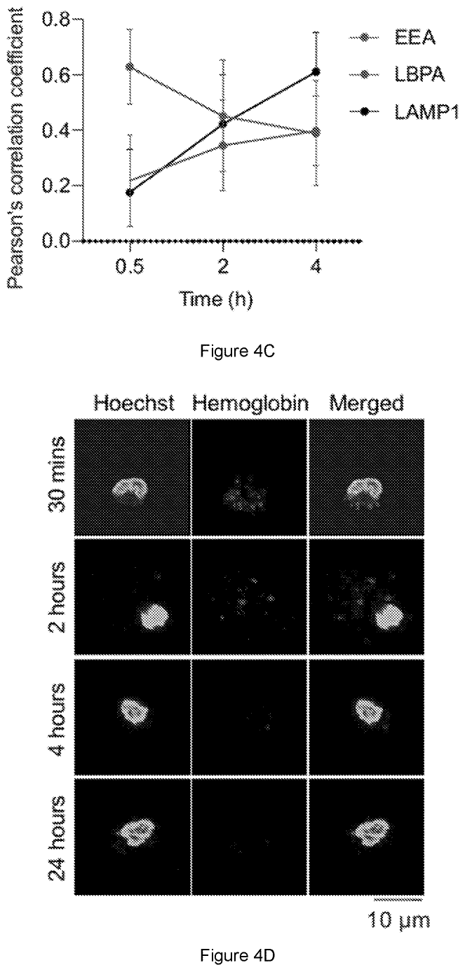

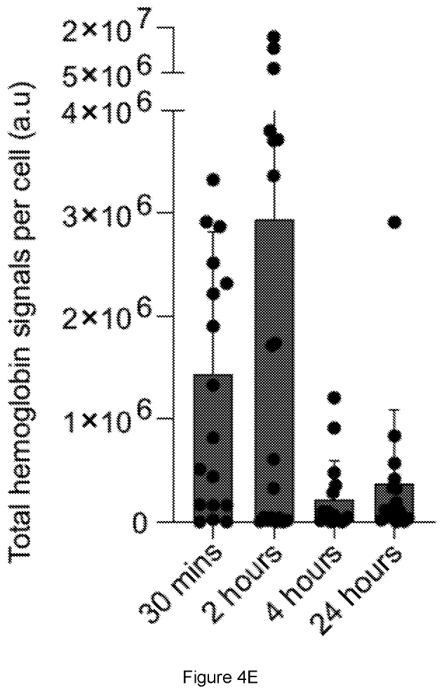

To track the fate of RBCEVs after being endocytosed, we incubated macrophages with CFSE-labeled RBCEVs for 0.5 hours, 2 hours, 4 hours, and 24 hours. RBCEVs were present in the medium till 2 hours for macrophages to uptake RBCEVs. After the first 2 hours, RBCEVs were removed and replaced with fresh medium to study degradation rate of cargo (FIG. 4a). Cells were then permeabilized and stained with early endosomal marker (EEA), late endosomal marker (LBPA), and lysosomal marker (LAMP1). Using Pearson's colocalization coefficient, we found that after adding RBCEVs for 30 minutes, CFSE signal mostly colocalized with LBPA signal (FIG. 4b). This colocalization with LBPA decreased over time and CFSE increasingly localized to LAMP1-positive endosomes. EEA showed a low Pearson's coefficient, implying poor colocalization overall, although the coefficient slightly increased with time (FIG. 4c). These data indicate that RBCEVs rapidly accumulated in late endosomes and progressively transitioned to lysosomes after 2-4 hours of incubation.

To track the degradation rate of hemoglobin, the most abundant protein in RBCEVs, we detected the presence of hemoglobin in macrophages at different time points by immunofluorescent staining of RBCEV-treated macrophages. Our data showed that after as early as 2 hours of incubation, most hemoglobin was degraded, resulting in disappearance of hemoglobin after 4 hours (2 hours after removing RBCEVs in the medium) (FIGS. 4d and 4e). These data suggest that after being endocytosed, RBCEVs are broken down rapidly within 2 hours in macrophages.

It was not previously appreciated how EVs are processed intracellularly over time after cellular uptake. The present disclosure shows that, as early as 30 minutes and at least up to 4 hours after cellular uptake, RBCEV signals predominantly colocalize with markers of late endosomes (e.g., LBPA) and lysosomes (e.g., LAMP1), with little to no colocalization with early endosome markers (e.g., EEA).

### Example 6: RBCEVs Induce PBMC-Derived Macrophages into an Mheme-Like Phenotype and Reduce their CD86 Expression

The present Example provides technologies for inducing an Mheme-like phenotype in PBMCs with RBCEV treatment. Among other things, the present Example provides technologies that influence gene expression in cells differentiating into macrophages. The present Example particularly provides technologies relating to uptake into human PBMCs (e.g., CD14+ cells, e.g., monocytes).

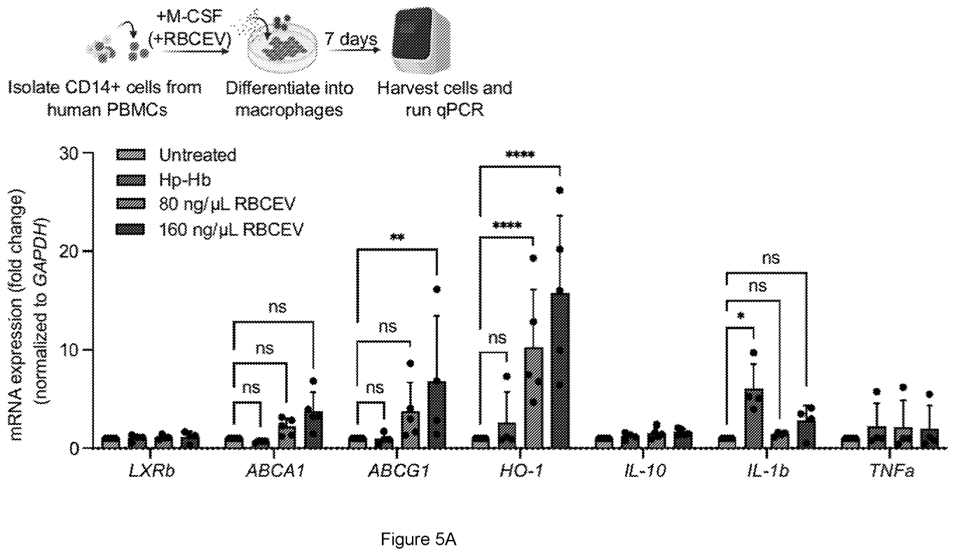

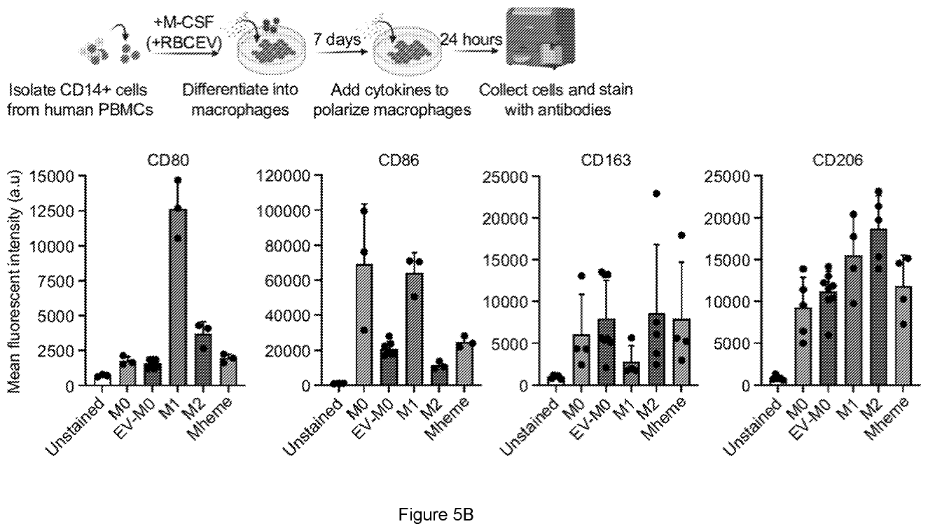

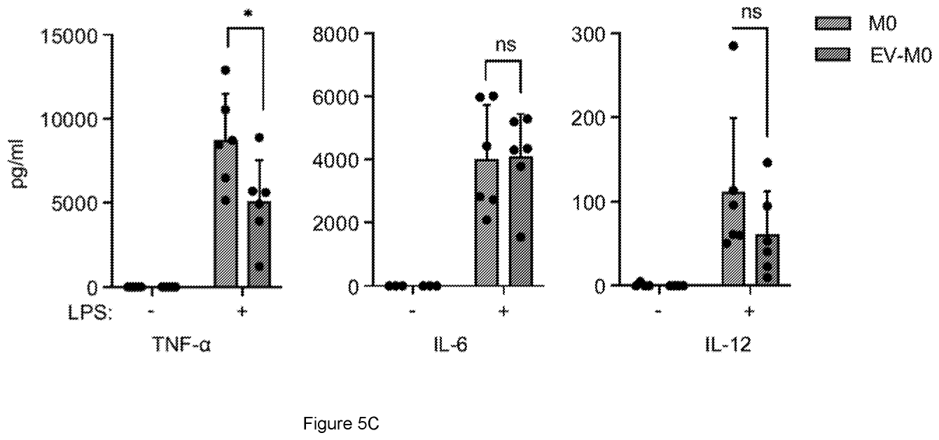

We assessed RBCEV impact on heme metabolism in macrophages. We added RBCEVs to CD14+ cells isolated from human PBMCs during their differentiation into macrophages (with M-CSF treatment), and quantified the expression of heme metabolizing enzyme Heme oxygenase 1 (HO-1). We found that treatment with 80-160 ng/μl RBCEVs significantly upregulated the expression of HO-1 mRNA in differentiating macrophages, relative to the negative control with M-CSF treatment only, and even higher than HO-1 expression in the positive control with haptoglobin and hemoglobin treatment in addition to M-CSF. Among other genes associated with heme metabolism, the expression of cholesterol efflux transporters ABCA1 and ABCG1 also increased in the RBCEV-treated group (FIG. 5a). The increase in the expression of HO-1, ABCA1, and ABCG1 resembles the Mheme phenotype of macrophages found in atherosclerosis lesions, which exhibit atheroprotective properties¹⁹. RBCEV treatment did not provoke gene expression of inflammatory cytokines, while a combination of haptoglobin and hemoglobin produces a slight increase in IL-1b expression (FIG. 5a).

Based on surface marker expression, we found that macrophages treated with RBCEVs and M-CSF displayed an Mheme and/or M2 phenotype more so than an M1 phenotype (FIG. 5b). Expression of M1 marker CD80 was low in RBCEV-induced macrophages. We also found a significant reduction in CD86 expression in RBCEV-induced macrophages compared to MO and M1 macrophages. This trend in CD86 reduction is similar in macrophages stimulated to the Mheme and M2 phenotypes. The expression of CD163 on macrophages induced by RBCEVs was not clear due to its variation among PBMCs from different donors. However, CD163 and CD206 were slightly increased in RBCEV-induced macrophages compared to MO macrophages (FIG. 5b).

We also quantified the levels of 4 common pro-inflammatory and anti-inflammatory cytokines in monocyte-derived macrophages using ELISA (FIG. 5c). Macrophages exposed to RBCEVs did not activate the inflammatory cytokines, as no TNF-α, IL-6, or IL-12 was detected in the supernatant. We further investigated whether RBCEVs could attenuate inflammation by challenging macrophages with LPS. Remarkably, monocyte-derived macrophages primed with RBCEVs prior to LPS stimulation significantly reduced TNF-α and slightly reduced IL-12 in the supernatant, demonstrating that RBCEVs are capable of attenuating excessive inflammatory signals in macrophages in response to LPS stimuli (FIG. 5c).

The present disclosure demonstrates that in vitro incubation with RBCEVs induces macrophages to adopt a similar phenotype to Mheme and M2 macrophages but distinct from M1 macrophages. This effect was characterized by downregulation of M1 marker CD86 and slight upregulation, albeit statistically insignificant, of CD163 and CD206. With regards to the hemoglobin metabolic pathways, we observed increased expression of HO-1, which encodes the protein that degrades heme, and increased expression of cholesterol export channel genes ABCA1 and ABCG1 in macrophages treated with RBCEVs although these increases were not significant in ABCA1 and ABCG1 at low dose (80 ng/μL) RBCEV treatment. These changes enable cells to reduce retention of oxLDL and thus become resistant to foam cell formation, similar to Mheme macrophages. For LPS-activated macrophages in particular, co-treatment with RBCEVs and a metal chelator (e.g., EDTA) enhanced the oxLDL efflux over RBCEV treatment alone. Together, our results highlight the highly promising potential of RBCEVs as natural therapeutic carriers of hemoglobin for atherosclerosis treatment and suggest their plausible co-administration with iron chelators for improved efficacy.

There are reports that particular types of EVs might have anti-inflammatory effects in certain instances. For example, EVs derived from human umbilical cord mesenchymal stem cells have shown protective effects when delivering peptide hydrogels to treat cardiac injuries³². The use of EVs from human adipose mesenchymal stem cells has also been successful in inhibiting LPS-activated monocytes via the delivery of miR-132 and miR-146a³³. However, it is expensive to culture mesenchymal stem cells as a source of production for EVs, which greatly limits the accessibility of anti-inflammatory EV preparations from this source. Costs would scale up over time and might become prohibitive in cases of managing chronic inflammation.

The present disclosure teaches that RBCEVs, in contrast, are distinguished in multiple ways, including cheaper and more efficient production, e.g., from blood samples, which are often available from blood banks.

The present disclosure furthermore demonstrates that RBCEVs have an endogenous capability to induce anti-inflammatory effects in macrophages. This is evidenced by a significant reduction in TNF-α secreted by LPS-activated macrophages, for example. RBCEVs did not provoke the mRNA expression of pro-inflammatory cytokine genes (e.g., IL-1b and TNF-α) in non-activated macrophages, suggesting their suitability and safety for use in managing inflammation.

### Example 7: Hemoglobin Carried by RBCEVs Induces Macrophages into an Mheme-Like Phenotype

The present Example provides technologies for inducing an Mheme-like phenotype in PBMCs with RBCEV treatment. Among other things, the present Example provides insight(s) that endogenous protein within RBCEVs (e.g., hemoglobin) can influence gene expression in cells differentiating into macrophages. The present Example particularly provides technologies relating to uptake into human PBMCs (e.g., CD14+ cells, e.g., monocytes).

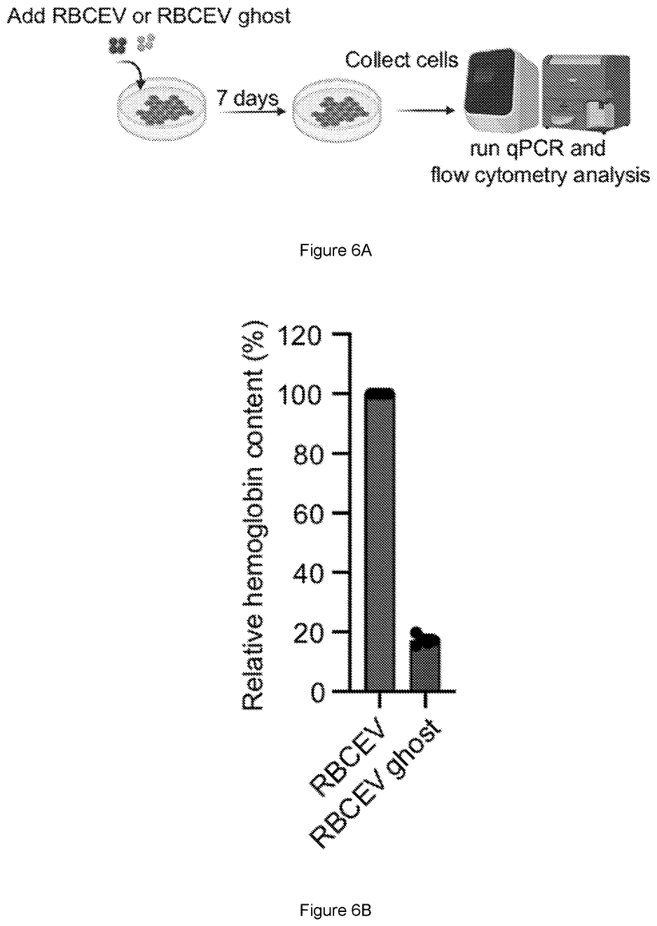

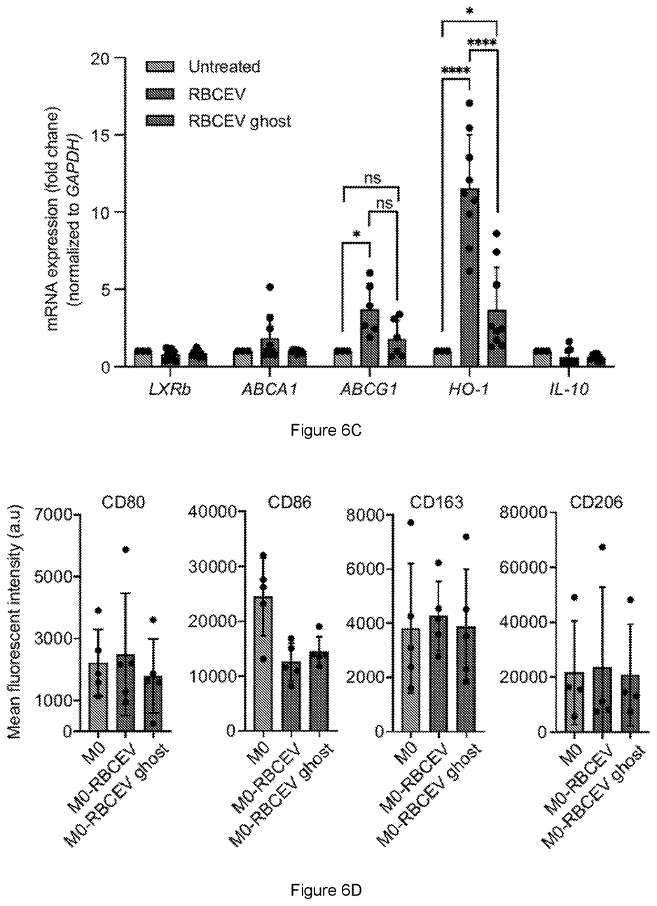

The present disclosure hypothesizes that hemoglobin delivered by RBCEVs can induce an Mheme-like phenotype. To test this hypothesis, we reduced the hemoglobin contents in RBCEVs by freezing and then thawing the EVs in water for 3 rounds to prepare RBCEV ghosts (FIGS. 6a and 6b). Human CD14+ PBMCs were incubated with equivalent amounts of RBCEV ghosts and RBCEVs during M-CSF treatment to differentiate into macrophages. After 7 days, expression of Mheme-associated genes and polarization surface markers were analyzed. HO-1 mRNA was significantly lower in macrophages treated with RBCEV ghosts compared to those treated with RBCEVs (FIG. 6c). These data suggest that hemoglobin plays an important role in inducing an Mheme-like phenotype in macrophages incubated with RBCEVs.

### Example 8: Induction of Mheme-Like Phenotype by RBCEVs is Mediated by the Heme Transporter HRG-1

The present Example provides technologies for inducing an Mheme-like phenotype in PBMCs with RBCEV treatment. Among other things, the present Example provides insight(s) that, upon uptake into cells, RBCEVs can influence phenotype through interactions with HRG-1. The present Example particularly provides technologies relating to uptake into macrophages.

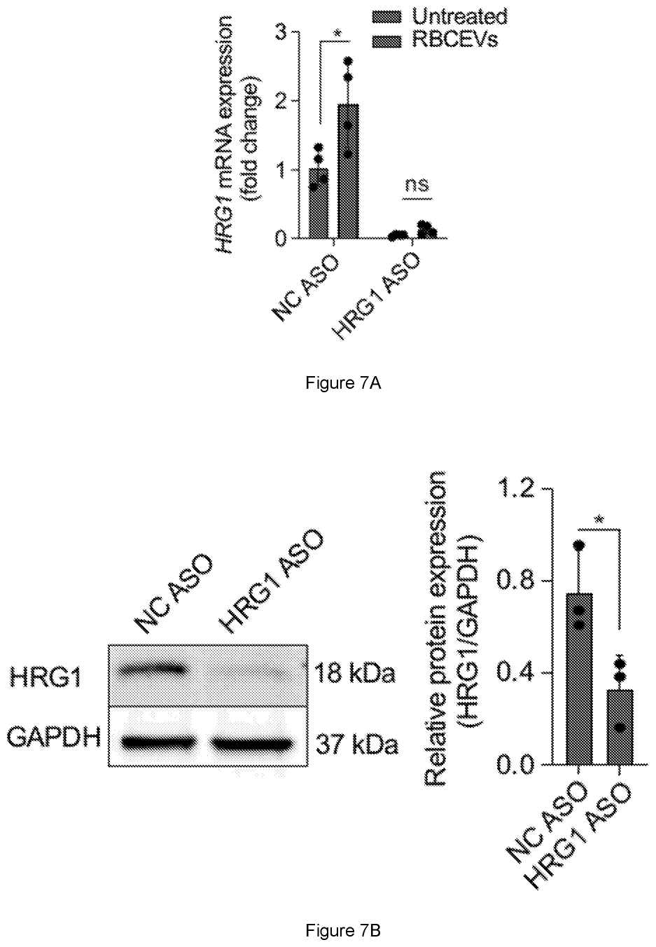

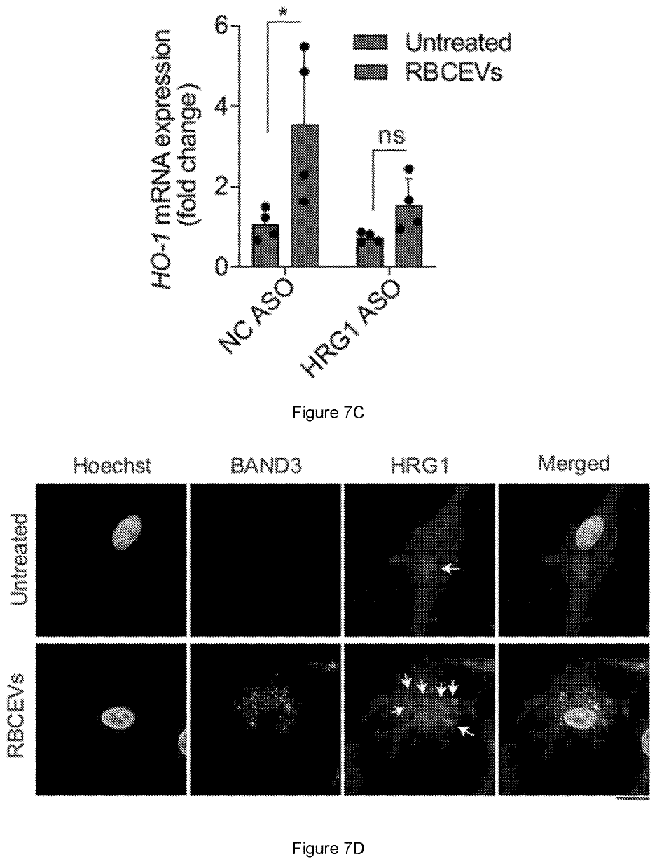

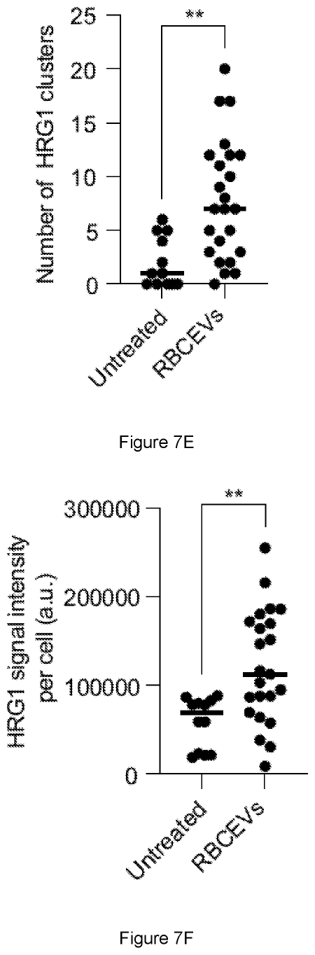

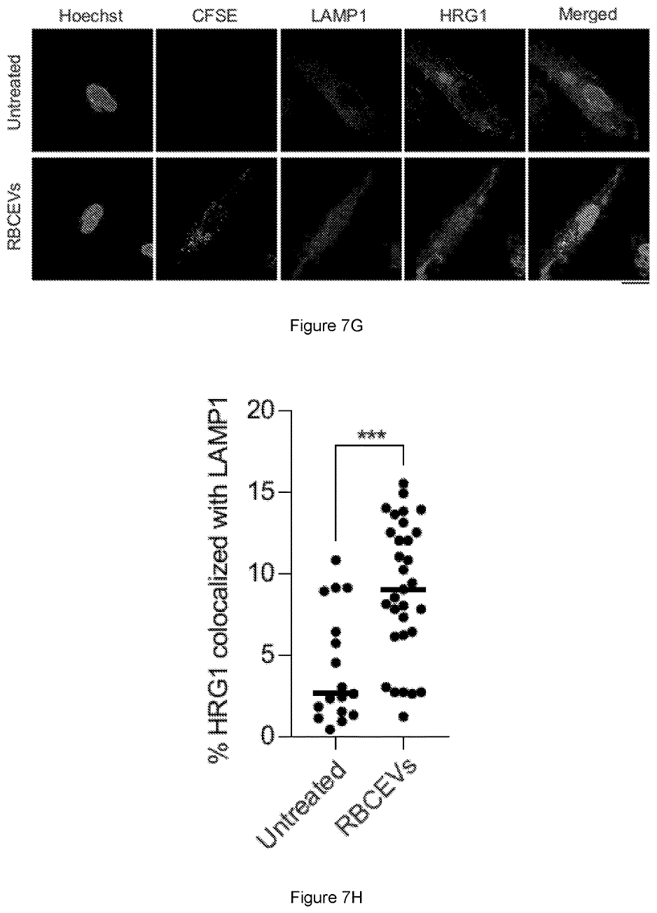

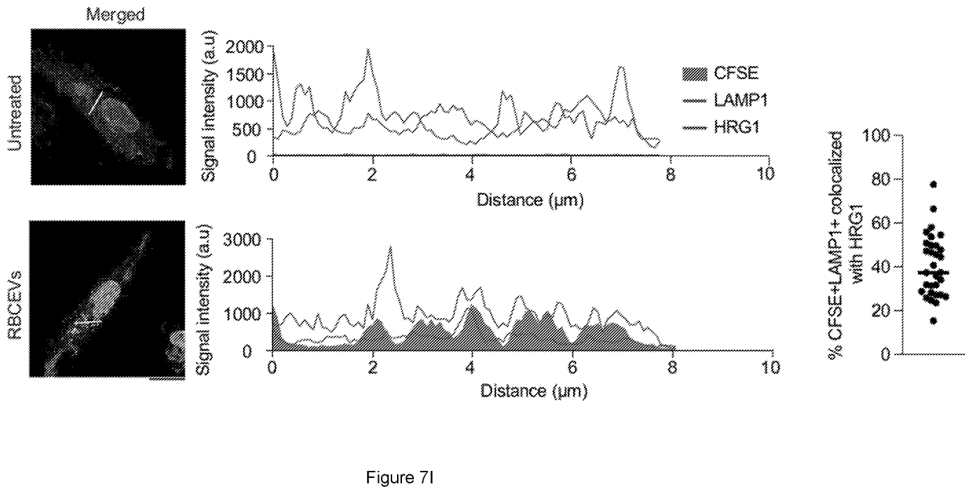

The present disclosure hypothesizes that hemoglobin contained within RBCEVs is likely responsible, at least in part, for inducing an Mheme-like phenotype in cells that have taken up RBCEVs. It has been observed that, upon hemoglobin degradation in late endosomes and lysosomes, heme can be released and transported across the endosomal membrane to the cytosol by heme transporter HRG-122. Heme in the cytosol can bind to its targets and induce changes associated with the Mheme phenotype, including upregulation of HO-119. We performed knockdown of HRG-1 using antisense oligonucleotides (ASOs). We designed and validated an HRG-1-targeting ASO which reduced expression of HRG-1 mRNA after 24 hours and HRG-1 protein after 72 hours (FIGS. 7a and 7b). Interestingly, incubation with RBCEVs for 2 hours following transfection with either HRG-1 ASO or NC ASO increased HRG-1 mRNA expression compared to untreated control, suggesting that RBCEVs upregulated HRG-1 expression. Nonetheless, even with addition of RBCEVs, HRG-1 ASO still exhibited high mRNA knockdown efficiency. More importantly, expression of HO-1 was markedly reduced following transfection with HRG-1 ASO compared with NC ASO treatment (FIG. 7c). These data support our hypothesis that release of heme from the endo/lysosomes via HRG-1 mediates differentiation of macrophages into an Mheme-like phenotype (a distinct phenotype, with an upregulation in HO-1 expression similar to Mheme macrophages).

To better understand the involvement of HRG1 in mediating the effects of RBCEVs, we analyzed changes in the cellular distribution of HRG1 after macrophages were incubated with RBCEVs for 2 hours. Notably, RBCEV treatment resulted in an increased number of HRG1 clusters per cell (FIGS. 7d and 7e). The HRG1 signal intensity per cell was also higher, possibly because the physical clustering of HRG1 produced more concentrated signals that were more distinguishable from the background noise (FIGS. 7d-f). Furthermore, a higher percentage of HRG1 signals appeared to overlap with LAMP1 signals in RBCEV-treated cells when compared with untreated controls. Intensity profiles revealed areas where CFSE signals (indicating RBCEVs) and LAMP1 signals (indicating lysosomes) co-localized with the HRG1 signals (FIGS. 7g-i). Together, these results support the idea that incubation with RBCEVs prompts HRG1 to cluster in macrophages and become distributed more to the lysosomes. This mechanism likely contributes to the functional export of RBCEV-derived heme via the HRG1 transporter from endo-lysosomes to the cytosol.

Without wishing to be bound by any particular theory, we note that it is plausible that once hemoglobin is degraded in macrophages (within 2 hours after RBCEV addition according to our data), heme is released from the endolysosomal system into the cytosol via the HRG-1 receptor, as has been demonstrated when senescent RBCs are processed by macrophages²². Importantly, we show that ASO-mediated knockdown of HRG-1 mRNA suppressed upregulation of HO-1 by treatment with RBCEVs, thus elucidating at least part of the mechanism by which RBCEVs induce an Mheme-like phenotype.

### Example 9: RBCEVs Reduce Foam Cell Formation from Macrophages

The present Example provides technologies for reducing and/or preventing foam cell formation with RBCEV treatment. Among other things, the present Example provides technologies for quantification and/or assessment of foam cell formation (e.g., oxLDL retention) with oil red O staining. The present Example particularly provides technologies relating to preventing and/or reducing foam cell formation of macrophages with RBCEV treatment.

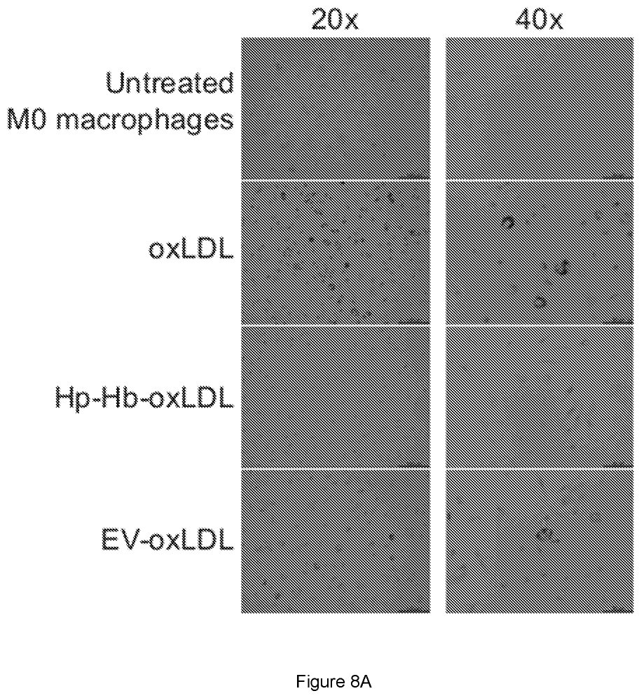

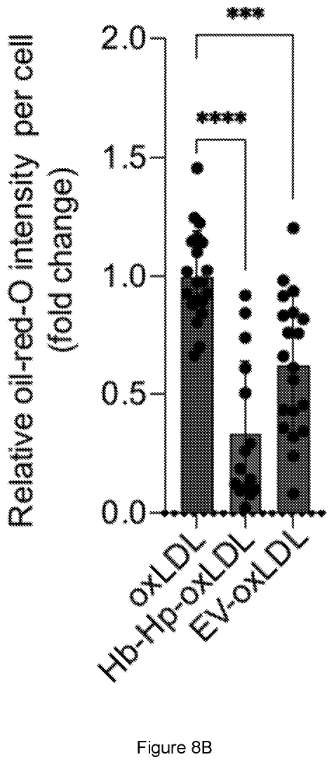

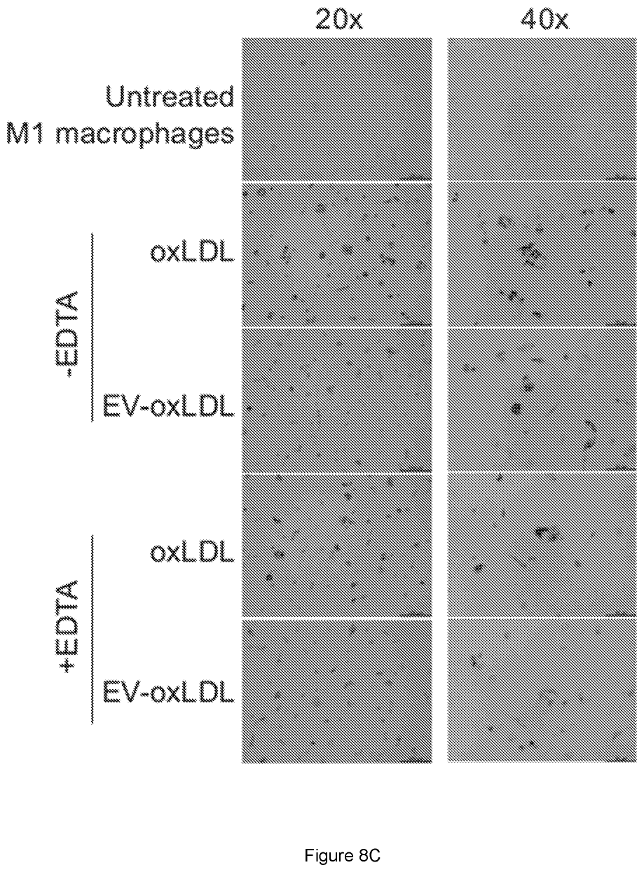

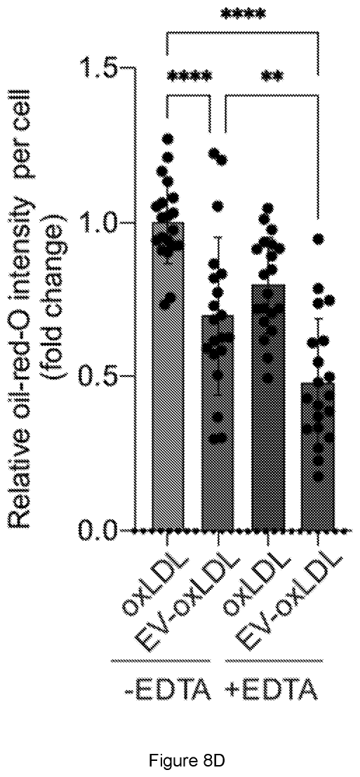

Having demonstrated that RBCEV-induced macrophages exhibited upregulated expression of ABCA1 and ABCG1-two genes encoding cholesterol efflux transporters, we assessed ability of RBCEVs to inhibit or prevent foam cell formation after challenging macrophages with oxLDL. This assessment was accomplished by comparing the level of oil red O staining as an indicator of oxLDL retention, which is elevated in foam cells relative to normal macrophages. As shown in FIGS. 8a and 8b, substantial levels of oil red O staining were observed in macrophages challenged with oxLDL, while treatment with hemoglobin-haptoglobin markedly reduced oil red O staining (FIGS. 8a and 8b). RBCEV-primed macrophages also showed significantly lower levels of oil red O staining, suggesting that RBCEVs can protect against foam cell formation and potentially atherosclerosis (FIG. 8b).

Classically activated M1 macrophages are considered to play a crucial role in atherosclerosis progression. We assessed the ability of RBCEVs to mitigate foam cell formation in such activated macrophages. Oil red O staining experiment was again performed with M1 activated macrophages, and we found that there was no significant reduction in foam cell formation in the RBCEV-treated group. However, as RBCEVs also deliver iron into the cells and given that iron accumulation might prevent protective effects against atherosclerosis^{23,24}, we tested co-treatment of the activated macrophages with both RBCEVs and the metal ion chelator EDTA. Indeed, this co-treatment produced the lowest level of oil red O-positive stained cells amongst the four groups (FIGS. 8c and 8d).

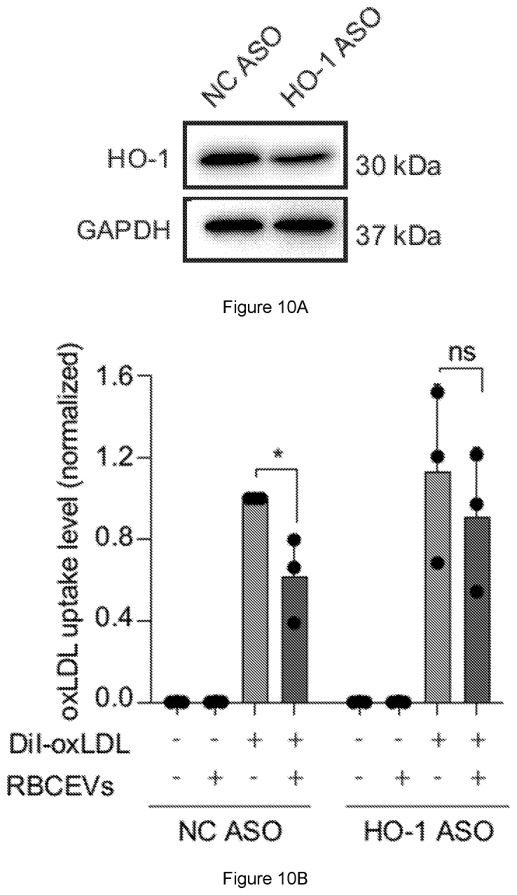

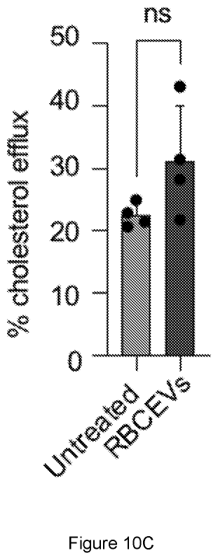

We also tested the involvement of HO-1 in the modulation of lipid uptake by knocking down HO-1 using an ASO. We confirmed that the protein expression of HO-1 was reduced 48 hours after the ASO transfection (FIG. 10a). We observed that in the group transfected with the HO-1 ASO, RBCEV treatment produced a smaller reduction in Dil-oxLDL uptake compared to the group transfected with an NC ASO (FIG. 10b). These results suggest a mechanism for reducing lipid uptake which involves the modulation of the HO-1 pathway by RBCEVs, and likely by the hemoglobin in the RBCEVs in particular. On the other hand, we did not find significant differences in the level of cholesterol efflux between the untreated and RBCEV-treated groups (FIG. 10c). Together, these data indicate that the reduction in foam cell formation resulted primarily from a decrease in uptake of lipids by macrophages rather than alteration in cholesterol efflux.

Even though Mheme macrophages are resistant to foam cell transformation and are in this way atheroprotective, evidence has emerged suggesting this macrophage phenotype might also contribute to plaque instability by promoting angiogenesis and vascular permeability. Data from clinical studies identified a significant correlation between Mheme presence and plaque progression as well as microvascularity. This is attributed to decreased iron retention in Mheme macrophages, which activates the HIF1alpha master switch and subsequently upregulates VEGF, leading to elevated angiogenesis and impaired endothelial integrity. Plaques thus become unstable, inflamed, and prone to rupturing²⁴. Observing similarity between RBCEV-induced and Mheme phenotypes, we propose that that RBCEVs could produce a similar phenomenon, which might warrant additional efforts to manage the undesirable effects. We have previously shown that RBCEVs can serve as robust delivery vehicles for RNA-based therapeutics, including siRNAs and ASOs, for efficient gene knockdown^{3,6,31}. We provide an insight that siRNAs against VEGF or its relevant downstream targets could be loaded into RBCEVs prior to administration to treat atherosclerosis. Successful execution of this strategy would allow RBCEVs to exert their protective effects on macrophages via hemoglobin-mediated signaling without causing incidental disruptions to the plaques.

### Example 10: RBCEVs Reduce Atherosclerotic Lesions in ApoE Knockout Mice on a High-Fat Diet

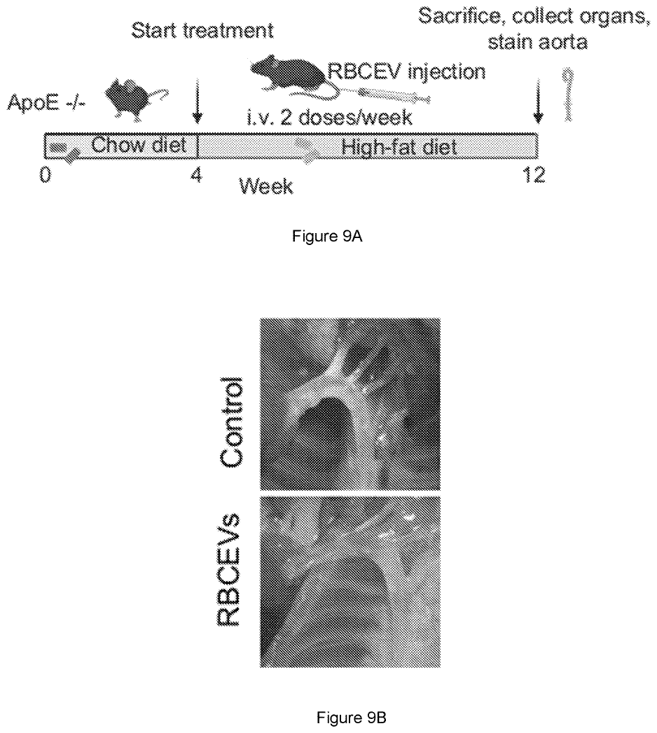

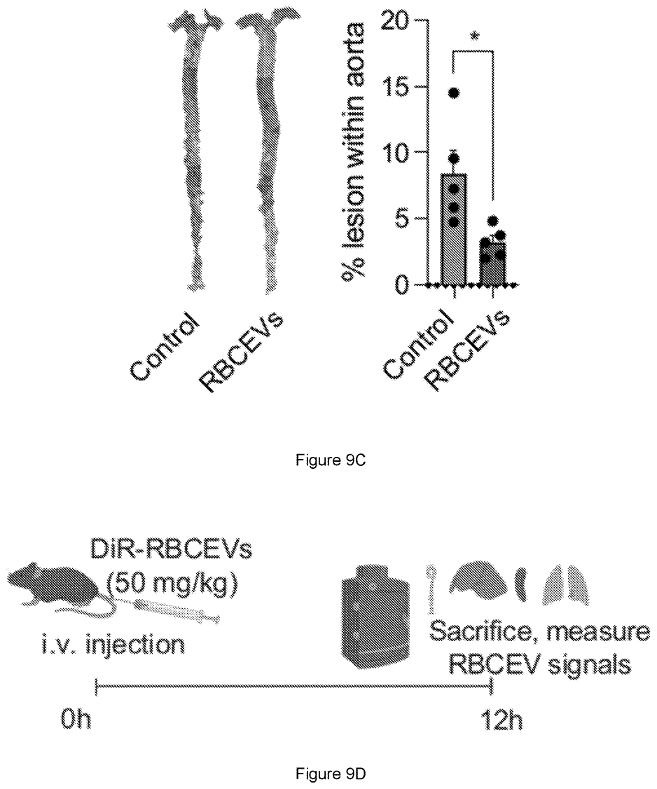

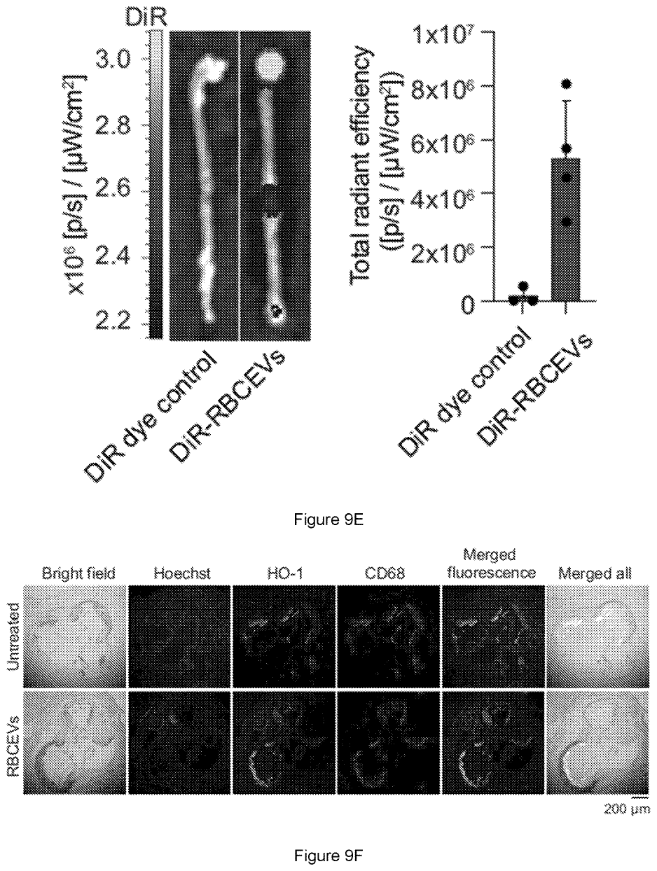

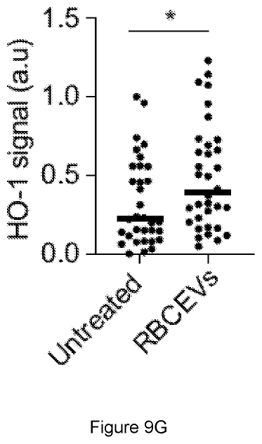

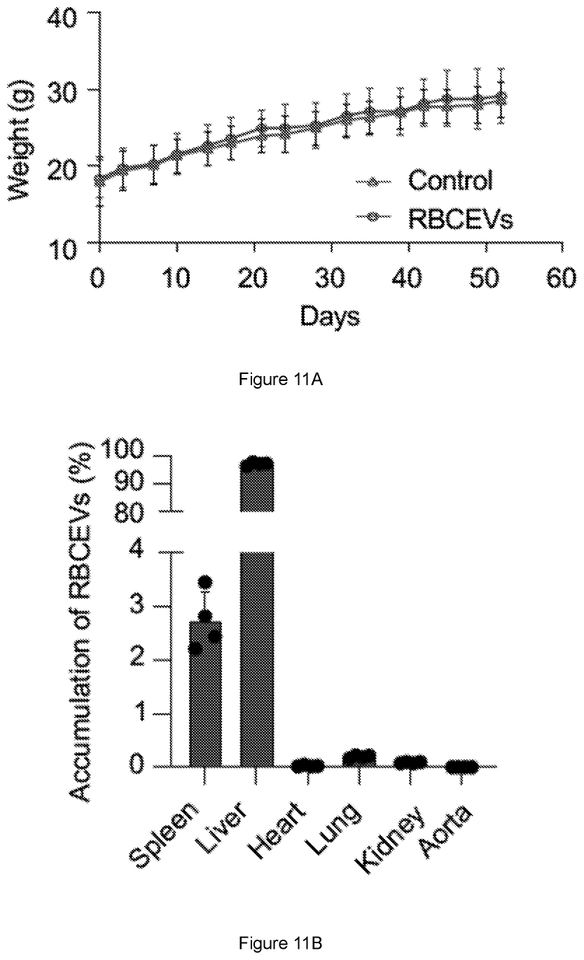

Having observed the reduction of foam cell formation in vitro, we went on to apply the RBCEV treatment to atherosclerosis mouse models. ApoE knockout (ApoE−/−) mice on a high-fat diet received RBCEVs administered via tail vein injection at the dose of 50 mg/kg twice a week. After 8 weeks, the entire aorta was collected and stained with ORO to assess the total lesion formation (FIG. 9a). Mice treated with RBCEVs showed a reduction in total aortic lesions (FIGS. 9b and 9c). In detail, the amount of atherosclerotic plaques, represented by the white spots, in the aortic arch decreased in RBCEV-treated mice (FIG. 9b). Quantification of the lesions by staining the entire aorta with ORO revealed a decrease in the total percentage of plaque area (FIG. 9c). Encouragingly, the body weight of the mice remained similar between the control and treated groups, suggesting that the RBCEV treatment was not toxic (FIG. 11a). Our data from the macrophage monoculture, organ-on-chip model, and animal model collectively demonstrate the preventive effect of RBCEVs on foam cell formation and atherosclerosis.

To determine if RBCEVs are distributed to the plaque area, RBCEVs were stained with the DiR dye. Similar concentrations of free DiR were used as a control group, and staining and washing procedures were performed on both the DiR-labeled EV samples and the control samples. RBCEVs and control dyes were administered via tail vein injection at 50 mg/kg. After 12 hours, the aortas were collected and analyzed using IVIS® Spectrum In Vivo Imaging System (FIG. 9d). The analysis revealed that although the vast majority of RBCEVs ended up in the liver as expected (FIG. 11b), there was a clear signal corresponding to DiR-labeled EVs in the plaque area (FIG. 9e).

We further investigated the expression of HO-1 in the aortic roots by co-staining with antibodies against HO-1 as well as the mouse macrophage marker CD68. At least ten sections from different areas of the aortic roots of mice in each treatment group were stained and quantified (see “Materials and methods” section). We observed tissue areas double-positive for HO-1 and CD68, indicating the expression of HO-1 in macrophages within the aortic roots (FIG. 9f). Importantly, we found that the overall HO-1 expression levels increased in mice treated with RBCEVs compared with the control group (FIG. 9g). Overall, our data in the ApoE−/− mouse model revealed the ability of RBCEVs to reduce atherosclerotic lesions and confirmed a concomitant increase in HO-1 expression in macrophages residing in the aortic root.

We appreciate desirability of targeting RBCEVs to atherosclerotic sites when treating atherosclerosis. As has been demonstrated, a large percentage of injected RBCEVs accumulate in the liver and spleen and are quickly cleared from circulation^{3,7}. Successful covalent conjugation of functional nanobodies and peptides onto RBCEVs, has also been demonstrated, as has its achievement of specific targeting to EGFR-positive cancer cells⁴. The present disclosure proposes that it is possible to attach nanobodies or peptides that recognize markers of injured tissues or oxidized lipids to RBCEV surface, so that these vesicles preferentially target and accumulate at therapeutic concentrations at atherosclerotic plaques.

Overall, the present Examples demonstrate robust endocytosis of RBCEVs by human monocyte-derived macrophages, leading to adoption of an Mheme-like phenotype that is resistant to foam cell transformation and is potentially anti-inflammatory. Coupled with the scalability of RBCEV production and the capacity of the RBCEV platform for further engineering and drug loading, these data are indicative of a novel strategy to suppress atherosclerosis progression.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

SEQUENCES

| SEQ ID | | |
|---|---|---|
| NO: | DESCRIPTION | SEQUENCE |
| 1 | GAPDH forward | GTCTCCTCTGACTTCAACAGCG |
| 2 | GAPDH reverse | ACCACCCTGTTGCTGTAGCCAA |
| 3 | ABCA1 forward | TGTCCAGTCCAGTAATGGTTCTGT |
| 4 | ABCA1 reverse | AAGCGAGATATGGTCCGGATT |
| 5 | ABCG1 forward | TGCAATCTTGTGCCATATTTGA |
| 6 | ABCG1 reverse | CCAGCCGACTGTTCTGATCA |
| 7 | HO-1 forward | CTTCACCTTCCCCAACATTG |
| 8 | HO-1 reverse | CTTGCAACTCCTCAAAGAGC |
| 9 | IL-1β forward | CCACAGACCTTCCAGGAGAATG |
| 10 | IL-1β reverse | GTGCAGTTCAGTGATCGTACAGG |
| 11 | IL-10 forward | CCTGCCTAACATGCTTCGAG |
| 12 | IL-10 reverse | CTCAGACAAGGCTTGGCAAC |
| 13 | LXRb forward | CTTCGCTAAGCAAGTGCCTGGT |
| 14 | LXRb reverse | CACTCTGTCTCGTGGTTGTAGC |
| 15 | TNF-α forward | CCTCTCTCTAATCAGCCCTCTG |
| 16 | TNF-α reverse | GAGGACCTGGGAGTAGATGAG |
| 17 | HRG1-ASO (5′->3′) | CCTCCAATAATCTTGCATGT |
| 18 | HO-1 ASO (5′-3′) | ATCACCAGCTTGAAGCCGTC |
| 19 | NC-ASO | CGACTATACGCGCAATATGG |
| 20 | HRG1-ASO (5′->3′) | +C*/12MOErC/*/i2MOErT/*/i2MOErC/* |
| | (showing | /i2MOErC/*A*A*T*A*A*T*C*T*T*G* |
| | modifications) | /12MOErC/*/i2MOErA/*/i2MOErT/* |
| | | /i2MOErG/*+T |
| 21 | HO-1 ASO (5′-3′) | +A*/i2MOErT/*/i2MOErC/*/12MOErA/* |
| | (showing | /i2MOErC/*C*A*G*C*T*T*G*A*A*G* |
| | modifications) | /12MOErC/*/12MOErC/*/i2MOErG/* |
| | | /i2MOErT/*+C |
| 22 | NC-ASO (showing | +C*/i2MOErG/*/i2MOErA/*/i2MOErC/* |
| | modifications) | /i2MOErT/*A*T*A*C*G*C*G*C*A*A* |
| | | /12MOErT/*/i2MOErA/*/i2MOErT/* |
| | | /i2MOErG/*+G |

Key:
/12MOEr/ = 21-OMe modification at internal nucleotide.
*= phosphorothioate (PS) modification

### REFERENCES CITED

1. Van Niel, G., d'Angelo, G. & Raposo, G. Shedding light on the cell biology of extracellular vesicles. Nature reviews Molecular cell biology 19, 213-228 (2018).
2. Herrmann, I. K., Wood, M. J. A. & Fuhrmann, G. Extracellular vesicles as a next-generation drug delivery platform. Nature nanotechnology 16, 748-759 (2021).
3. Usman, W. M. et al. Efficient RNA drug delivery using red blood cell extracellular vesicles. Nat Commun 9, 2359 (2018).
4. Pham, T. C. et al. Covalent conjugation of extracellular vesicles with peptides and nanobodies for targeted therapeutic delivery. Journal of Extracellular Vesicles 10, (2021).
5. Jayasinghe, M. K. et al. Surface-engineered extracellular vesicles for targeted delivery of therapeutic RNAs and peptides for cancer therapy. Theranostics 12, 3288-3315 (2022).
6. Peng, B. et al. Robust delivery of RIG-I agonists using extracellular vesicles for anti-cancer immunotherapy. J of Extracellular Vesicle 11, (2022).
7. Zhang, G. et al. Extracellular vesicles: Natural liver-accumulating drug delivery vehicles for the treatment of liver diseases. Journal of Extracellular Vesicles 10, (2020).
8. Jeney, V. et al. Pro-oxidant and cytotoxic effects of circulating heme. Blood, The Journal of the American Society of Hematology 100, 879-887 (2002).
9. Fabriek, B. O., Dijkstra, C. D. & van den Berg, T. K. The macrophage scavenger receptor CD163. Immunobiology 210, 153-160 (2005).
10. Araujo, J. A., Zhang, M. & Yin, F. Heme oxygenase-1, oxidation, inflammation, and atherosclerosis. Frontiers in pharmacology 3, 119 (2012).
11. Kishimoto, Y., Kondo, K. & Momiyama, Y. The protective role of heme oxygenase-1 in atherosclerotic diseases. International journal of molecular sciences 20, 3628 (2019).
12. Otterbein, L. E. et al. Carbon monoxide has anti-inflammatory effects involving the mitogen-activated protein kinase pathway. Nature medicine 6, 422-428 (2000).
13. Stocker, R., Yamamoto, Y., McDonagh, A. F., Glazer, A. N. & Ames, B. N. Bilirubin is an antioxidant of possible physiological importance. Science 235, 1043-1046 (1987).
14. Ishikawa, K., Navab, M. & Lusis, A. J. Vasculitis, atherosclerosis, and altered HDL composition in heme-oxygenase-1-knockout mice. International journal of hypertension 2012, (2012).
15. Juan, S.-H. et al. Adenovirus-mediated heme oxygenase-1 gene transfer inhibits the development of atherosclerosis in apolipoprotein E-deficient mice. Circulation 104, 1519-1525 (2001).
16. Ishikawa, K. et al. Heme oxygenase-1 inhibits atherosclerotic lesion formation in LDL-receptor knockout mice. Circulation Research 88, 506-512 (2001).
17. Finn, A. V. et al. Hemoglobin directs macrophage differentiation and prevents foam cell formation in human atherosclerotic plaques. Journal of the American College of Cardiology 59, 166-177 (2012).
18. Orozco, L. D. et al. Heme oxygenase-1 expression in macrophages plays a beneficial role in atherosclerosis. Circulation research 100, 1703-1711 (2007).
19. Boyle, J. J. et al. Coronary intraplaque hemorrhage evokes a novel atheroprotective macrophage phenotype. The American journal of pathology 174, 1097-1108 (2009).
20. Zhou, C. et al. Conjugated Oligoelectrolytes for Long-Term Tumor Tracking with Incremental NIR-II Emission. Advanced Materials 2201989 (2022).
21. Zhou, C. et al. A Chain-Elongated Oligophenylenevinylene Electrolyte Increases Microbial Membrane Stability. Advanced Materials 31, 1808021 (2019).
22. White, C. et al. HRG1 Is Essential for Heme Transport from the Phagolysosome of Macrophages during Erythrophagocytosis. Cell Metabolism 17, 261-270 (2013).
23. Guo, L., Harari, E., Virmani, R. & Finn, A. V. Linking Hemorrhage, Angiogenesis, Macrophages, and Iron Metabolism in Atherosclerotic Vascular Diseases. ATVB 37, (2017).
24. Guo, L. et al. CD163+ macrophages promote angiogenesis and vascular permeability accompanied by inflammation in atherosclerosis. Journal of Clinical Investigation 128, 1106-1124 (2018).
25. Costa Verdera, H., Gitz-Francois, J. J., Schiffelers, R. M. & Vader, P. Cellular uptake of extracellular vesicles is mediated by clathrin-independent endocytosis and macropinocytosis. Journal of Controlled Release 266, 100-108 (2017).
26. Heusermann, W. et al. Exosomes surf on filopodia to enter cells at endocytic hot spots, traffic within endosomes, and are targeted to the ER. Journal of Cell Biology 213, 173-184 (2016).
27. Parolini, I. et al. Microenvironmental pH Is a Key Factor for Exosome Traffic in Tumor Cells. Journal of Biological Chemistry 284, 34211-34222 (2009).
28. Joshi, B. S., de Beer, M. A., Giepmans, B. N. G. & Zuhorn, I. S. Endocytosis of Extracellular Vesicles and Release of Their Cargo from Endosomes. ACS Nano 14, 4444-4455 (2020).
29. Buzás, E. I., Tóth, E. Á., Sódar, B. W. & Szabó-Taylor, K. É. Molecular interactions at the surface of extracellular vesicles. in vol. 40 453-464 (Springer, 2018).
30. Mulcahy, L. A., Pink, R. C. & Carter, D. R. F. Routes and mechanisms of extracellular vesicle uptake. Journal of extracellular vesicles 3, 24641 (2014).
31. Chen, H. et al. CD33-targeting extracellular vesicles deliver antisense oligonucleotides against FLT3-ITD and MIR-125b for specific treatment of acute myeloid leukaemia. Cell Proliferation 55, (2022).
32. Han, C. et al. Human umbilical cord mesenchymal stem cell derived exosomes encapsulated in functional peptide hydrogels promote cardiac repair. Biomater. Sci. 7, 2920-2933 (2019).
33. Heo, J. S. & Kim, S. Human adipose mesenchymal stem cells modulate inflammation and angiogenesis through exosomes. Sci Rep 12, 2776 (2022).
34. Huynh, M.-L. N., Fadok, V. A. & Henson, P. M. Phosphatidylserine-dependent ingestion of apoptotic cells promotes TGF-β1 secretion and the resolution of inflammation. The Journal of clinical investigation 109, 41-50 (2002).
35. Harel-Adar, T. et al. Modulation of cardiac macrophages by phosphatidylserine-presenting liposomes improves infarct repair. Proceedings of the National Academy of Sciences 108, 1827-1832 (2011).
36. Mills, C. M1 and M2 macrophages: oracles of health and disease. Critical Reviews™ in Immunology 32, (2012).
37. Chinetti-Gbaguidi, G., Colin, S., & Staels, B. Macrophage subsets in atherosclerosis. Nature Reviews Cardiology 12.1, 10-17 (2015).

What is claimed is:

1. A method of treating and/or preventing an inflammatory disease, disorder, or condition in a human subject comprising administering to the subject a composition comprising a population of extracellular vesicles derived from red blood cells (RBCEVs).

2. A composition comprising a population of extracellular vesicles derived from red blood cells (RBCEVs) for use in a method of treating and/or preventing an inflammatory disease, disorder, or condition.

3. The use of a composition comprising a population of extracellular vesicles derived from red blood cells (RBCEVs) in the manufacture of a medicament for treating and/or preventing an inflammatory disease, disorder, or condition.

4. The method, composition for use or use of any one of claims 1 to 3, wherein the RBCEVs comprise heme, hemoglobin and/or phosphatidylserine.

5. The method, composition for use or use of any one of claims 1 to 4, wherein the RBCEVs are not loaded with exogenous nucleic acid.

6. The method, composition for use or use of any one of claims 1 to 4, wherein the RBCEVs are loaded with exogenous nucleic acid.

7. The method, composition for use or use of claim 6, wherein the exogenous nucleic acid is or comprises an siRNA or an ASO for the gene knockdown of VEGF.

8. The method, composition for use or use of any one of claims 1-7, wherein the inflammatory disease, disorder, or condition is or comprises atherosclerosis.

9. The method of any one of claims 1-8, characterized in that the administration of the composition comprising a population of RBCEVs is associated with reduced levels of one or more inflammatory cytokines.

10. The method of claim 9, wherein the inflammatory cytokines are selected from the group consisting of TNF-α, IL-6, and IL-12.

11. The method of any one of claims 1-8, characterized in that the administration of the composition comprising a population of RBCEVs is associated with reduced formation of foam cells.

12. The method of any one of claims 1-8, characterized in that the administration of the composition comprising a population of RBCEVs is associated with increased induction of Mheme-like phenotype in macrophages.

13. A pharmaceutical composition comprising a population of extracellular vesicles derived from red blood cells (RBCEVs) for the treatment and/or prevention of atherosclerosis.
